SEQUENCE LISTING (70 sequences)

SEQ ID NO: 1 - DNA, 1200 bp, Homo sapiens
atgccctggagtagtcgcggggctctcctgcgggaccttgtgctgggagtactcgggacagcggcgttcctgttggacctcggaactgacttgtgggccgccgtccagtacgcacttggtggaaggtacctttgggcggcgctggtcctggccctcttggggctggcaagcgtcgctctccagctctttagctggctgtggcttcgcgcagatcccgctgggctgcatgggtcccagccgccaaggagatgcctggctctgctccatcttctccagctcgggtatctttacagatgcgtacaagagttgcgccagggccttcttgtttggcaacaagaggaaccaagtgagttcgacctcgcctatgcggatttccttgcgttggatatctccatgcttcggctcttcgaaacattccttgagaccgcgccacaattgacccttgtacttgcaatcatgctgcaatctggacgagcagaatactaccaatgggtgggaatctgcacatccttcctgggcatcagttgggccctccttgattatcatcgcgccttgagaacttgtttgccaagcaaaccattgttgggcctcggatcctctgttatttattttctctggaatctgctgcttttgtggccgcgagtactcgctgttgcgcttttttccgcgttgttcccttcctacgtcgcgctccattttctcggcctgtggctggttctgctgttgtgggtttggctgcaagggacggactttatgccagacccgtccagtgagtggctttaccgggttacagttgcgaccatactttatttctcctggtttaatgtcgcagagggacgaactcgcgggagagccataatccacttcgcattcctcctctcagattcaatactcctggtcgccacctgggtaacacactcatcatggctcccaagtgggatacctttgcaattgtggttgccggttggctgcgggtgtttcttcctgggtctcgctcttagacttgtctattatcattggctgcacccgagttgctgctggaagcctgacccggtgggacctgattttggtagagaattcgcgcggtccttgctctccccagaaggctaccagttgccccaaaatagacgcatgactcaccttgcccagaagttctttcccaaagccaaggacgaggcagcttctcctgtcaaggggtag

SEQ ID NO: 2 - protein, 399 aa, Homo sapiens
MPWSSRGALLRDLVLGVLGTAAFLLDLGTDLWAAVQYALGGRYLWAALVLALLGLASVALQLFSWLWLRADPAGLHGSQPPRRCLALLHLLQLGYLYRCVQELRQGLLVWQQEEPSEFDLAYADFLALDISMLRLFETFLETAPQLTLVLAIMLQSGRAEYYQWVGICTSFLGISWALLDYHRALRTCLPSKPLLGLGSSVIYFLWNLLLLWPRVLAVALFSALFPSYVALHFLGLWLVLLLWVWLQGTDFMPDPSSEWLYRVTVATILYFSWFNVAEGRTRGRAIIHFAFLLSDSILLVATWVTHSSWLPSGIPLQLWLPVGCGCFFLGLALRLVYYHWLHPSCCWKPDPVGPDFGREFARSLLSPEGYQLPQNRRMTHLAQKFFPKAKDEAASPVKG

SEQ ID NO: 3 - DNA, 27 bp, Artificial Sequence
  Description of Artificial Sequence Synthetic oligonucleotide
gtgggacctgattttggtagagaattc

SEQ ID NO: 4 - protein, 9 aa, Artificial Sequence
  Description of Artificial Sequence Synthetic peptide
VGPDFGREF

SEQ ID NO: 5 - DNA, 1212 bp, Artificial Sequence
  Description of Artificial Sequence Synthetic polynucleotide
atgccctggagtagtcgcggggctctcctgcgggaccttgtgctgggagtactcgggacagcggcgttcctgttggacctcggaactgacttgtgggccgccgtccagtacgcacttggtggaaggtacctttgggcggcgctggtcctggccctcttggggctggcaagcgtcgctctccagctctttagctggctgtggcttcgcgcagatcccgctgggctgcatgggtcccagccgccaaggagatgcctggctctgctccatcttctccagctcgggtatctttacagatgcgtacaagagttgcgccagggccttcttgtttggcaacaagaggaaccaagtgagttcgacctcgcctatgcggatttccttgcgttggatatctccatgcttcggctcttcgaaacattccttgagaccgcgccacaattgacccttgtacttgcaatcatgctgcaatctggacgagcagaatactaccaatgggtgggaatctgcacatccttcctgggcatcagttgggccctccttgattatcatcgcgccttgagaacttgtttgccaagcaaaccattgttgggcctcggatcctctgttatttattttctctggaatctgctgcttttgtggccgcgagtactcgctgttgcgcttttttccgcgttgttcccttcctacgtcgcgctccattttctcggcctgtggctggttctgctgttgtgggtttggctgcaagggacggactttatgccagacccgtccagtgagtggctttaccgggttacagttgcgaccatactttatttctcctggtttaatgtcgcagagggacgaactcgcgggagagccataatccacttcgcattcctcctctcagattcaatactcctggtcgccacctgggtaacacactcatcatggctcccaagtgggatacctttgcaattgtggttgccggttggctgcgggtgtttcttcctgggtctcgctcttagacttgtctattatcattggctgcacccgagttgctgctggaagcctgacccgggatcggtgggacctgattttggtagagaattcggcagtgcgcggtccttgctctccccagaaggctaccagttgccccaaaatagacgcatgactcaccttgcccagaagttctttcccaaagccaaggacgaggcagcttctcctgtcaaggggtag

SEQ ID NO: 6 - protein, 403 aa, Artificial Sequence
  Description of Artificial Sequence Synthetic polypeptide
MPWSSRGALLRDLVLGVLGTAAFLLDLGTDLWAAVQYALGGRYLWAALVLALLGLASVALQLFSWLWLRADPAGLHGSQPPRRCLALLHLLQLGYLYRCVQELRQGLLVWQQEEPSEFDLAYADFLALDISMLRLFETFLETAPQLTLVLAIMLQSGRAEYYQWVGICTSFLGISWALLDYHRALRTCLPSKPLLGLGSSVIYFLWNLLLLWPRVLAVALFSALFPSYVALHFLGLWLVLLLWVWLQGTDFMPDPSSEWLYRVTVATILYFSWFNVAEGRTRGRAIIHFAFLLSDSILLVATWVTHSSWLPSGIPLQLWLPVGCGCFFLGLALRLVYYHWLHPSCCWKPDPGSVGPDFGREFGSARSLLSPEGYQLPQNRRMTHLAQKFFPKAKDEAASPVKG

SEQ ID NO: 7 - DNA, 39 bp, Artificial Sequence
  Description of Artificial Sequence Synthetic oligonucleotide
ggatcggtgggacctgattttggtagagaattcggcagt

SEQ ID NO: 8 - protein, 13 aa, Artificial Sequence
  Description of Artificial Sequence Synthetic peptide
GSVGPDFGREFGS

SEQ ID NO: 9 - DNA, 2178 bp, Homo sapiens
gagggctgcgcccacctccttcctgcctcggcaaccccgggccctgagggcaggccccaaccgcggaggagcaggagagggcggaggccggcgggccatgccctggtcgtcccgcggcgccctccttcgggacctggtcctgggcgtgctgggcaccgccgccttcctgctcgacctgggcaccgacctgtgggccgccgtccagtatgcgctcggcggccgctacctgtgggcggcgctggtgctggcgctgctgggcctggcctccgtggcgctgcagctcttcagctggctctggctgcgcgctgaccctgccggcctgcacgggtcgcagcccccgcgccgctgcctggcgctgctgcatctcctgcagctgggttacctgtacaggtgcgtgcaggagctgcggcaggggctgctggtgtggcagcaggaggagccctctgagtttgacttggcctacgccgacttcctcgccctggacatcagcatgctgcggctcttcgagaccttcttggagacggcaccacagctcacgctggtgctggccatcatgctgcagagtggccgggctgagtactaccagtgggttggcatctgcacatccttcctgggcatctcgtgggcactgctcgactaccaccgggccttgcgcacctgcctcccctccaagccgctcctgggcctgggctcctccgtgatctacttcctgtggaacctgctgctgctgtggccccgagtcctggctgtggccctgttctcagccctcttccccagctatgtggccctgcacttcctgggcctgtggctggtactgctgctctgggtctggcttcagggcacagacttcatgccggaccccagctccgagtggctgtaccgggtgacggtggccaccatcctctatttctcctggttcaacgtggctgagggccgcacccgaggccgggccatcatccacttcgccttcctcctgagtgacagcattctcctggtggccacctgggtgactcatagctcctggctgcccagcgggattccactgcagctgtggctgcctgtgggatgcggctgcttctttctgggcctggctctgcggcttgtgtactaccactggctgcaccctagctgctgctggaagcccgaccctgaccaggtagacggggcccggagtctgctttctccagaggggtatcagctgcctcagaacaggcgcatgacccatttagcacagaagtttttccccaaggctaaggatgaggctgcttcgccagtgaagggataggtgaacggcgtcctttgaagcaggatcagacccagccagcagagatggagagtgactctgttggcagaaggcaggcgaggataagctaacgatgctgctgtggcctctatgcactcagcaagagcgggacgcctgtgctgggccgggcaccagggatggtgctgagtcgggcagaggcctcctttcaaggagttcacagtgaacaagatgagaagggctgggccctggagggtcaagagccccaattatgtacaagacactttgggaggaaagaagactaccttttccccctgccattggtatagctggtgccccaaaacttccacctccctccctggctacctctaaaatgactggtataggtgctgccccaccccttagctcccctatcctgggctaggaggccacaggggctgtcctctagaattcttccttccctcccccacaccattcattcaattcatgaaacaaatctttgccaagagcagtttatgtgccaggaacatcattctgtccttgcaacctggaacaagaccagctaccagcctagcttcatccgctacttgcaccaaccagtcccgggttagatcccaaatgctagaagccagggatgcccaactctgggtggccccagtcagaacctctgggatctcagtgaagctggcctggcctctgctcctgctctcaaggggctgcttttcaaccaagagccttgtgagcctggtctgagccttgcacagccactgagtattttttttgccttagccagtgtacctcctacctcagtctatgtgagaggaagagaatgtgtgtgcctgtgggtctctacaagtgacagatgtgttgttttcaacagtattattaggttatgaataaagcctcatgaaatcctc

SEQ ID NO: 10 - protein, 395 aa, Homo sapiens
MPWSSRGALLRDLVLGVLGTAAFLLDLGTDLWAAVQYALGGRYLWAALVLALLGLASVALQLFSWLWLRADPAGLHGSQPPRRCLALLHLLQLGYLYRCVQELRQGLLVWQQEEPSEFDLAYADFLALDISMLRLFETFLETAPQLTLVLAIMLQSGRAEYYQWVGICTSFLGISWALLDYHRALRTCLPSKPLLGLGSSVIYFLWNLLLLWPRVLAVALFSALFPSYVALHFLGLWLVLLLWVWLQGTDFMPDPSSEWLYRVTVATILYFSWFNVAEGRTRGRAIIHFAFLLSDSILLVATWVTHSSWLPSGIPLQLWLPVGCGCFFLGLALRLVYYHWLHPSCCWKPDPDQVDGARSLLSPEGYQLPQNRRMTHLAQKFFPKAKDEAASPVKG

SEQ ID NO: 11 - DNA, 4238 bp, Mus musculus
gacgactgccccgcccccttcctgccggactagcggggcgggagggcaggtccgcggttgtgtggttgcttggagaggatcatgcctctgtccgtgcaccaccatgtggccttagacgtggtcgtaggcctggtgagtatcttgtctttcctgctggatctggtcgctgacctgtgggccgttgtccagtacgtgctccttggccgttatctgtgggccgcgctggtactggtcctgctgggccaagcttcggtgctgctgcagctcttcagctggctctggctgacagctgatcccaccgagctgcaccattcgcagctctcgcgtcctttcctggctctgctgcacctgctgcagctcggctacctgtataggtgtttgcacggaatgcatcaagggctgtccatgtgctaccaggagatgccatccgagtgtgacctggcctacgcagactttctctccctggacatcagcatgctgaagcttttcgagagcttcctggaggcgacgccacagctcacactggtgctggcaattgtattgcagaatggccaggcggaatactaccagtggtttggcatcagctcatcctttcttggcatctcgtgggcactgctggattaccatcggtctctgcgtacctgtcttccctccaagccacgcctgggccggagttcctctgctatctacttcctgtggaacctgctgctgctggggcccagaatctgtgccatcgccttgttctcagctgtcttcccctactatgtggccctgcatttcttcagcctgtggctggtacttttgttctggatctggcttcaaggcacaaattttatgcctgactccaaaggtgagtggctgtaccgggtgacaatggccctcatcctctatttctcctggttcaacgtgtctgggggccgcactcgaggccgggccgtcatccacctgatcttcatcttcagtgacagtgttctgctggtcaccacctcctgggtgacacacggcacctggctgcccagtgggatctcattgctgatgtgggtgacaataggaggagcctgcttcttcctgggactggctttgcgtgtgatctactacctctggctgcaccctagctgcagctgggaccctgacctcgtggatgggaccctaggactcctttctccccatcgtcctcctaagctgatttataacaggcgtgccaccctgttagcagagaacttcttcgccaaggccaaagctcgggctgtcctgacagaggaggtgcagctgaatggagtcctctgaggcagggtctgattcagccagtgaggaagataatgcgagtggggccttgcaagggacaaggcgggccagtcatgtgcaagccattttttttcttctgaagccgatggaactgctgtcagcaaacactcggttgtttgttgttctcacctctcaggtgattggtggcgtcctggctcctggttccctagcccgctctagatgacacaagattctgggagaactcttccctaccccatcccatccattcacttcaaccaacaaatgctaaaggcactttatgttctgggaacaccatcctggcttctgaactgcctgccactctagcttctttccctgcccacctggacagatcctgggtagactcctaaacagtgaggccaggtatgtccctccagtgtcctgatgctcaggccacctttataccaagtgccttatggacctgtggtctaggccatgtgatgcccagtaagtattttcattctcctacctcagtctatgtggaagaacatatatgcatgtgtttaacagtattaaagcctcatgagattctccagaccagtatgtaccactaagtgtagtctatcaccctttacagacacgtagaaggcgcctggaaccccttaaaactgacacagacccctggcatacaaatgtgggcataggtttgacttaattttgcttcccaagacgcaggggctagtgagcccgagccggttgatcattcggctagcagaactcatgggcagatgctagtgtattcttttagcagctccgtactgagcctaaagaggacttgaggatggggatggcaggtttgaggggctggatggaaggtaaaggattgggggttctttttgggtgagaggtgcagtggcttctgggatgtggtcaatagctccgtggaggtggcgtgttctgctctcggaggtttgtggtcttgttgggaaaagggaacaggagagaggctccaggggcagaagaaaaggttccaggtcccagtgctgggacccagatagttctagcagtcattcatttatttgtgtggacgtgaaataacctgtgacccaaacaagcaccaagtactgaaagaaaaccagatggagaggtgagagggaggatgtatgttgtgggtggaagttgcagctttataaaaaaccattggggaggacccctctgagaaactgaggcatagactgtaagctacttcagcagtgactgcagcatggagtctgcgtggtttgttggagaaggaatctgcgaatgctgttccctgtggcacagcaaccccactgtaagaggactgtggggtgcggttggctcacagccaaggaggctgcagagatgcaggtgggggcctggaagaggctctgggagaaggtacttcttatactaaaaggtacaggctgactatggacagaaaggacctaatttccagacctgaattttacagaccaggaaaaggagccaaagtggttgttgatgttaaaagggtctgaaaaacagtcaccacctccgtgttcactctcatggaaaaacggatgtaatcacaccagaaggtgtcatcctctaaacagatgcccccacaggtacacacctgaaatcactgttactctcatttatgaaaatggtaagatagggatgagccagtgtgacacacctaccagtctgggcaaggacatcaggagttcagactcctcagtgacaatgtcagaggccagcttgggctacatgagaccctgtctccaacaaaatgaaattattttatttatttatttatttggctttttgagacggggtttctctgtgtagccctggctgtcctggaactcactctgtagaccaggctatcctcaaactcagaaatctgcctgcctctgcctcccaagtgctgggattaaaggcatgcgccaccacgcctggcacattttttttttaaattaaaaaaagaaagacgttactaccctgctcttgttttgtgacacacaatctggtctgagaggaccctgagcacatcttccttccttcaacactaccgtgctaagttcttaaaatctgggacttaaaaccaggttagtgacattacccgtagttaggatgtttggtttgttggggattggttctaatgctctgtcttaattcggctcccagaatcacacgggaatctgctctgctaaaggaagcctgtcactagttggctgtgattgggaaataaagttgcccagggctggctgggcaggaaagaggcgggacttttaggttgtgagggcaaggaaccccggggagttggaagcagagggatttcactgcgcagttgggtctggggcagcagagatgaaatgatgacttagcaagtcgactcagggaggttaggggggtagaatgtatgctagtcgcacggagggttagacacgtccagccactgagctagtcagagcatatcaaagttagatggtgtgtgtctctcattcacaaatcccgggaacacttggccagccgggagtcaggggtctaagcactacagggtttggaaaccagccaacactagaatctgcacttgtgactgagcaggggtacggacaacagctaacagtctacttgagctgcactgcggctcagaagatcacttcccggagaaaattcaccttggagtccgacatatctcacctttggaagctagaaacaacttctaatttccttcactggaacaatgggtaaaaagccctcttgtaagctagtgggggccaatcagaccaaatgtggcagaatgtagaacacctggttggtgggacgggaagtcaggatttattgggttgcggcttaattaatgctcagcacagactgactcctccttggtaacgttcagcacactcgacagctctgaaatccattccatttctataccttaaaaagcagtgtattttagaaacaattcaaataaacatttctctcgc

SEQ ID NO: 12 - protein, 401 aa, Mus musculus
MPLSVHHHVALDVVVGLVSILSFLLDLVADLWAVVQYVLLGRYLWAALVLVLLGQASVLLQLFSWLWLTADPTELHHSQLSRPFLALLHLLQLGYLYRCLHGMHQGLSMCYQEMPSECDLAYADFLSLDISMLKLFESFLEATPQLTLVLAIVLQNGQAEYYQWFGISSSFLGISWALLDYHRSLRTCLPSKPRLGRSSSAIYFLWNLLLLGPRICAIALFSAVFPYYVALHFFSLWLVLLFWIWLQGTNFMPDSKGEWLYRVTMALILYFSWFNVSGGRTRGRAVIHLIFIFSDSVLLVTTSWVTHGTWLPSGISLLMWVTIGGACFFLGLALRVIYYLWLHPSCSWDPDLVDGTLGLLSPHRPPKLIYNRRATLLAENFFAKAKARAVLTEEVQLNGVL

SEQ ID NO: 13 - DNA, 2671 bp, Rattus norvegicus
tgtgaggacgtctgccgaagggagcatgtgtgcgccatacagcacgtggagttcgacacttacgccacctgcttgcatggtcttggtgccaacctggtacctggtttcctgctcatactgactctgctgacgagcctacacgtattggaggtgctatgactgtaggcactgccagcctaccctcttacttggttcgtctttctccctggtaaaactgggcaacattacccaatggagagagagggagatgaattttgccatcagtctgtggagagtaaggtgggatgggacatttggattcaccagagagggcgctaagaagcacatttcttctgagttttatgttttatccacagagcttgtttgcggtacatgtcttggtgcattattccctttaatacaaacatcaaactatcatgcacttgatcgccacagtaaagtgaacccgcaggaagatgggccctggagagtctgtgcttttgagtccctgctcaaggtctaaaactgggaacccacgtggtctgcaaaatcccttggtacttttaaataaaagacttttctgatttggtttcgcaacagtgcaaccgtgagggatcacagctgcgacccagacactagtcttgtggccactcttgttaactagagcctcaaaaggcagaatccaaaccagtagaggcagggctcaagacagggagggctgggggcggggtctgggcggtgggaccgcctagggggcggagtcgtggactcgctcctccccggacggggcgagatggggaagttccgcccagcagcccggcctctgggaggactgccccacccccttcctgccggactagccgggctggagggcagatccgcggttgtgaggttgcctggagggccatgcctctgtccgtgcacccccaagtggccttagacgtggtcataggtctggtgagtaccttgtctttcctgttggacctggtcgccgacctgtgggccgtcgtccagtacgtgctcgttggccgttacctgtgggccgcgctggtagtggtgctgctgggccaagcctcggtgctgctgcagctcttcagctggctctggctgacagctgaccccaccgagctgcaccagttgcagccctcgcgtcgtttcctggctctgctgcacctgctgcagctcggctacctgtataggtgcctgcacggaatgcggcagggactgtccatgtgctgccaggaggtaccgtctgaatgtgacctggcctatgctgacttcctctccctggacatcagcatgctgcggctttttgagagcttcttggaggcgaccccacagctcacgctggtgctggccatcgtgttgcagagtggaaatgccgaatactaccagtggtttggcatcagctcatcctttctgggcatctcgtgggcattgctggactaccatcggtccttgcgcacctgcctcccctccaagccgcgcctgggctggtgctcctctgcggtctacttcctgtggaacctgctgctgttggggccccggatctgtgccatcgccacgttctcggtcgtctttccctactgcttggccctgcatttcctcagcctgtggctggtgctgttgtactgggtctggcttcaagacacgaagtttatgccaaactctaatggcgagtggctataccgggtgacggtggcgctcatcctttatttctcctggttcaatgtgtctgggggtcgcactcgaggccgggccactatccacctgggcttcatcctcagtgacagtgttctgcttgtcaccacctcctgggtgacagatagtacctggttgcccggtggggtcttattgtgggcggctttaggcggcgcctgcttctccctgggactggttttgcgtatgatctactacctccggctgcaccctagctgcagctgggaacccgactttgtggatgggaccctaagactcctccctcccgagcgtcctccaaagctgatttataacaggcgtgccactcggttagcacagaacttctttgccaagctcaaaacccaggccgccctgccacaggcggtacagctgaacggagtcctctgaggcagggtctgattcagccagtgaggaagatgaggagagtggggccttgcaagggacaagggggccaatcatgtgcaagccagtttttttcctctccaaccgatagagcttccattgccaaatcttcagttgttaccactttcacctctcacgtgattggtggcgtcctggttcctggttccctagcctgctctagatgacagactctgggggatgttctcgagaactcttccctaacctatcccatccattcacttcccccaacaaatgcactgatgttctgggagcatcatcctgacttctgaactggctgccaccctagcttctttccctgcccacctggacaaatcctccgtagactcttgaagagcggagggaggccagagatgcccctccagtgtcctgacgttcaggctcttaggccaccttacaccaagtgccttatggacctgtggcctaggccatgtgatgcccaccaagtatttttcattctcctacctcagtctgtgtgaaagaagaacatgtgtgcatgtgtttaacagtattaaaacctcacgagagtctccaaaaaaaaaaaaaaaaaaaa

SEQ ID NO: 14 - protein, 399 aa, Rattus norvegicus
MPLSVHPQVALDVVIGLVSTLSFLLDLVADLWAVVQYVLVGRYLWAALVVVLLGQASVLLQLFSWLWLTADPTELHQLQPSRRFLALLHLLQLGYLYRCLHGMRQGLSMCCQEVPSECDLAYADFLSLDISMLRLFESFLEATPQLTLVLAIVLQSGNAEYYQWFGISSSFLGISWALLDYHRSLRTCLPSKPRLGWCSSAVYFLWNLLLLGPRICAIATFSVVFPYCLALHFLSLWLVLLYWVWLQDTKFMPNSNGEWLYRVTVALILYFSWFNVSGGRTRGRATIHLGFILSDSVLLVTTSWVTDSTWLPGGVLLWAALGGACFSLGLVLRMIYYLRLHPSCSWEPDFVDGTLRLLPPERPPKLIYNRRATRLAQNFFAKLKTQAALPQAVQLNGVL

SEQ ID NO: 15 - DNA, 3200 bp, Homo sapiens
agaggtcacgtgacgccgcgcgggctgcgcgggcagtggtgggaaggctggcgcgaggcgtgaggtggcgtgaggcgaagctggaatctgcctctgtcacgggggctggtgcctcacgggtttgtgtcctagacaggcgagtggatccaagtgggcgagagacattttaatctggaagagtcttgtgatttgggagacagtgaagaagaagtaaaatattcacaagatgaagatttttccagaagggactttgagtcaaagatggctttttatatttgacaagtcttgtcatctgtaatgaagatcattgtgaaacagaagattgattaaagccttgtaacattggacctagattagagatttagaaaagaaagtcaaaattagtcactttagtgttagtgttcccatttcataatatttattctttcttctaaatagatttagggagtagaaattaaaattcaatgctataccaaagggtatactaatatttgtttggctttttttccctttttgtgagggagaaaaaagtagataacgaaaagctatagtcattcgtaatgaaatatactaaacagaattttatgatgtcagttcttggcattataatctacgtaactgatttaattgtggacatatgggtatctgtcagatttttccatgaaggacagtatgtttttagtgctttagcgttaagctttatgctttttggaacacttgtggctcagtgttttagttattcttggttcaaggctgatttaaagaaagcaggccaagaaagtcagcattgttttcttctacttcattgcttgcaaggaggagtttttacaaggtattggtttgccttaaaaaggggttaccatgcagcttttaaatatgacagcaatactagtaacttcgtggaagaacaaattgatctacataaagaagttatagatagagtgactgatttgagcatgctcagactatttgagacctacctggaaggctgcccacaacttattcttcaactctacattcttctggagcatggacaagcgaatttcagtcagtatgcggccatcatggtctcttgctgtgctatttcttggtcaactgttgattatcaagtagctttaagaaaatccttgcctgacaaaaagcttcttaatggattatgtcccaaaatcacatatctcttttacaagttgtttacattattatcgtggatgctgagtgttgtacttctactattcttaaatgttaagattgctttatttctgttgttatttctttggttgttaggtataatatgggcatttaaaaacaacacccagttttgtacttgtataagtatggaattcttatataggattgttgttggattcattcttatctttacattttttaatattaagggacagaataccaagtgtccaatgtcttgttattatattgttagggtactgggcactttggggatattgactgtattctgggtttgccccctcactatttttaatccagactattttatacctatcagtataactatagttcttactcttcttcttggaattctttttcttattgtttattatgggagttttcacccaaacagaagtgcagaaacaaaatgtgatgaaattgatggaaaaccagttctaagagaatgtagaatgagatatttcctaatggaataagctattcatttatgatatatattttcttatattttgtttcattggttagtaaagaaaatgtgtgttatgtgggtgtgttgtctcttatttttgccacctttaatttgaaattagttcagtgaaataggagatacatagtagtattttatttttaaaattaatttctcatttggttttgaagatcttgagtactcagatatctttctactgcctggtagagctgccatcttgagcctgaaatataagaaatggtctggttttcataatgagaaggctggaattgagcttccctcccattttccttgttcctgaactaatactactgtacctgttatggaggactgcaaagggaagagaaaagcagaacactgtattattttttcctttattgtcttcagtgcatatatttgcagttggggacaggttgagtagaggaaaagggaaagaagggaaagcagaaaacaaatttttagcatctgctgtgctttcatccatgaaatctccaattcagtaagtgcaaaagagaattggtgtgcatctgagaggtctgacatttcattatttacttatttcctagcttttctgaattaatgcactcttaacatataattatattaatcctatttgtgctagaatagttgtatctaaatcatattttaaaattatttttatttttaaaaaattatggtaaaaacatataaaatttaccatcttaatcactttgagtgtacagttcatcagtgttaactgtattcaccttgtgcaacagatctcaaggactttttcaccttgtaaaactaagattctctatttattgaacaaatccccatttcctccttccccaagtctctctcaactgaaattataattttttgtttctatgagtttgaatactttagataccttgttgccatggtttgaatgtgccccccagatttcatgtgtgtgaaacttaatctccaaatttgtatgttgatggcatttggaagtggtggggactttgtttatttatttatttttaattttttaattttatattattattattattattatactttaaggtttagggtacatgtgcacaatgtgcaggttagttacatatgtatacatgtgccatgctggtgtgctgcacccattaactcgtcatttatcattaggtatatctcctaaagctatccctcccccctccccccaccccacaacagtccccagagtgtgatgatccccttcctgtgtccatgtgttctcattgttcagttcccacctatgagtgagaatatgcagtgtttggttttttgttcttgcgatagtttactgagaatgatgatttccagcttcatccatgtccctacaaaggacatgaactcatcattttttatggctgcatagtattccatggtgtatatgtgccacattttcttaatccagtctattgttgttggacatttgggttggttccaagtctttgctattgtgaatagtgctgcaataaacatacgtgtgcatgtgtcttta

SEQ ID NO: 16 - DNA, 3318 bp, Homo sapiens
agaggtcacgtgacgccgcgcgggctgcgcgggcagtggtgggaaggctggcgcgaggcgtgaggtggcgtgaggcgaagctggaatctgcctctgtcacgggggctggtgcctcacgggtttgtgtcctagacaggcgagtggatccaagtgggcgagagacattttaatctggaagagtcttgtgatttgggagacagtgaagaagaagtaaaatattcacaagatgaagatttttccagaagggactttgagtcaaagatggctttttatatttgacaagtcttgtcatctgtaatgaagatcattgtgaaacagaagattgattaaagccttgtaacattggacctagattagagatttagaaaagaaagtcaaaattagtcactttagtgttagtgttcccatttcataatatttattctttcttctaaatagatttagggagtagaaattaaaattcaatgctataccaaagggtatactaatatttgtttggctttttttccctttttgtgagggagaaaaaagtagataacgaaaagctatagtcattcgtaatgaaatatactaaacagaattttatgatgtcagttcttggcattataatctacgtaactgatttaattgtggacatatgggtatctgtcagatttttccatgaaggacagtatgtttttagtgctttagcgttaagctttatgctttttggaacacttgtggctcagtgttttagttattcttggttcaaggctgatttaaagaaagcaggccaagaaagtcagcattgttttcttctacttcattgcttgcaaggaggagtttttacaagggccttgctctgtcacccaggctggcctgcagtggcgccttcccagctcattgcagcctccacctccttggttcaagagattctcctgcatcagcttcctgagtagctgggattacaggtattggtttgccttaaaaaggggttaccatgcagcttttaaatatgacagcaatactagtaacttcgtggaagaacaaattgatctacataaagaagttatagatagagtgactgatttgagcatgctcagactatttgagacctacctggaaggctgcccacaacttattcttcaactctacattcttctggagcatggacaagcgaatttcagtcagtatgcggccatcatggtctcttgctgtgctatttcttggtcaactgttgattatcaagtagctttaagaaaatccttgcctgacaaaaagcttcttaatggattatgtcccaaaatcacatatctcttttacaagttgtttacattattatcgtggatgctgagtgttgtacttctactattcttaaatgttaagattgctttatttctgttgttatttctttggttgttaggtataatatgggcatttaaaaacaacacccagttttgtacttgtataagtatggaattcttatataggattgttgttggattcattcttatctttacattttttaatattaagggacagaataccaagtgtccaatgtcttgttattatattgttagggtactgggcactttggggatattgactgtattctgggtttgccccctcactatttttaatccagactattttatacctatcagtataactatagttcttactcttcttcttggaattctttttcttattgtttattatgggagttttcacccaaacagaagtgcagaaacaaaatgtgatgaaattgatggaaaaccagttctaagagaatgtagaatgagatatttcctaatggaataagctattcatttatgatatatattttcttatattttgtttcattggttagtaaagaaaatgtgtgttatgtgggtgtgttgtctcttatttttgccacctttaatttgaaattagttcagtgaaataggagatacatagtagtattttatttttaaaattaatttctcatttggttttgaagatcttgagtactcagatatctttctactgcctggtagagctgccatcttgagcctgaaatataagaaatggtctggttttcataatgagaaggctggaattgagcttccctcccattttccttgttcctgaactaatactactgtacctgttatggaggactgcaaagggaagagaaaagcagaacactgtattattttttcctttattgtcttcagtgcatatatttgcagttggggacaggttgagtagaggaaaagggaaagaagggaaagcagaaaacaaatttttagcatctgctgtgctttcatccatgaaatctccaattcagtaagtgcaaaagagaattggtgtgcatctgagaggtctgacatttcattatttacttatttcctagcttttctgaattaatgcactcttaacatataattatattaatcctatttgtgctagaatagttgtatctaaatcatattttaaaattatttttatttttaaaaaattatggtaaaaacatataaaatttaccatcttaatcactttgagtgtacagttcatcagtgttaactgtattcaccttgtgcaacagatctcaaggactttttcaccttgtaaaactaagattctctatttattgaacaaatccccatttcctccttccccaagtctctctcaactgaaattataattttttgtttctatgagtttgaatactttagataccttgttgccatggtttgaatgtgccccccagatttcatgtgtgtgaaacttaatctccaaatttgtatgttgatggcatttggaagtggtggggactttgtttatttatttatttttaattttttaattttatattattattattattattatactttaaggtttagggtacatgtgcacaatgtgcaggttagttacatatgtatacatgtgccatgctggtgtgctgcacccattaactcgtcatttatcattaggtatatctcctaaagctatccctcccccctccccccaccccacaacagtccccagagtgtgatgatccccttcctgtgtccatgtgttctcattgttcagttcccacctatgagtgagaatatgcagtgtttggttttttgttcttgcgatagtttactgagaatgatgatttccagcttcatccatgtccctacaaaggacatgaactcatcattttttatggctgcatagtattccatggtgtatatgtgccacattttcttaatccagtctattgttgttggacatttgggttggttccaagtctttgctattgtgaatagtgctgcaataaacatacgtgtgcatgtgtcttta

SEQ ID NO: 17 - DNA, 3310 bp, Homo sapiens
agaggtcacgtgacgccgcgcgggctgcgcgggcagtggtgggaaggctggcgcgaggcgtgaggtggcgtgaggcgaagctggaatctgcctctgtcacgggggctggtgcctcacgggtttgtgtcctagacaggcgagtggatccaagtgggcgagagacattttaatctggaagagtcttgtgatttgggagacagtgaagaagaagtaaaatattcacaagatgaagatttttccagaagggactttgagtcaaagatggctttttatatttgacaagattcaaaatctagtgcattagacttttgaactagctgttccttcaagctggaaggcttttccatctctatgcacatggccaatttcactactcaaatgccaccttctcagtcttgtcatctgtaatgaagatcattgtgaaacagaagattgattaaagccttgtaacattggacctagattagagatttagaaaagaaagtcaaaattagtcactttagtgttagtgttcccatttcataatatttattctttcttctaaatagatttagggagtagaaattaaaattcaatgctataccaaagggtatactaatatttgtttggctttttttccctttttgtgagggagaaaaaagtagataacgaaaagctatagtcattcgtaatgaaatatactaaacagaattttatgatgtcagttcttggcattataatctacgtaactgatttaattgtggacatatgggtatctgtcagatttttccatgaaggacagtatgtttttagtgctttagcgttaagctttatgctttttggaacacttgtggctcagtgttttagttattcttggttcaaggctgatttaaagaaagcaggccaagaaagtcagcattgttttcttctacttcattgcttgcaaggaggagtttttacaaggtattggtttgccttaaaaaggggttaccatgcagcttttaaatatgacagcaatactagtaacttcgtggaagaacaaattgatctacataaagaagttatagatagagtgactgatttgagcatgctcagactatttgagacctacctggaaggctgcccacaacttattcttcaactctacattcttctggagcatggacaagcgaatttcagtcagtatgcggccatcatggtctcttgctgtgctatttcttggtcaactgttgattatcaagtagctttaagaaaatccttgcctgacaaaaagcttcttaatggattatgtcccaaaatcacatatctcttttacaagttgtttacattattatcgtggatgctgagtgttgtacttctactattcttaaatgttaagattgctttatttctgttgttatttctttggttgttaggtataatatgggcatttaaaaacaacacccagttttgtacttgtataagtatggaattcttatataggattgttgttggattcattcttatctttacattttttaatattaagggacagaataccaagtgtccaatgtcttgttattatattgttagggtactgggcactttggggatattgactgtattctgggtttgccccctcactatttttaatccagactattttatacctatcagtataactatagttcttactcttcttcttggaattctttttcttattgtttattatgggagttttcacccaaacagaagtgcagaaacaaaatgtgatgaaattgatggaaaaccagttctaagagaatgtagaatgagatatttcctaatggaataagctattcatttatgatatatattttcttatattttgtttcattggttagtaaagaaaatgtgtgttatgtgggtgtgttgtctcttatttttgccacctttaatttgaaattagttcagtgaaataggagatacatagtagtattttatttttaaaattaatttctcatttggttttgaagatcttgagtactcagatatctttctactgcctggtagagctgccatcttgagcctgaaatataagaaatggtctggttttcataatgagaaggctggaattgagcttccctcccattttccttgttcctgaactaatactactgtacctgttatggaggactgcaaagggaagagaaaagcagaacactgtattattttttcctttattgtcttcagtgcatatatttgcagttggggacaggttgagtagaggaaaagggaaagaagggaaagcagaaaacaaatttttagcatctgctgtgctttcatccatgaaatctccaattcagtaagtgcaaaagagaattggtgtgcatctgagaggtctgacatttcattatttacttatttcctagcttttctgaattaatgcactcttaacatataattatattaatcctatttgtgctagaatagttgtatctaaatcatattttaaaattatttttatttttaaaaaattatggtaaaaacatataaaatttaccatcttaatcactttgagtgtacagttcatcagtgttaactgtattcaccttgtgcaacagatctcaaggactttttcaccttgtaaaactaagattctctatttattgaacaaatccccatttcctccttccccaagtctctctcaactgaaattataattttttgtttctatgagtttgaatactttagataccttgttgccatggtttgaatgtgccccccagatttcatgtgtgtgaaacttaatctccaaatttgtatgttgatggcatttggaagtggtggggactttgtttatttatttatttttaattttttaattttatattattattattattattatactttaaggtttagggtacatgtgcacaatgtgcaggttagttacatatgtatacatgtgccatgctggtgtgctgcacccattaactcgtcatttatcattaggtatatctcctaaagctatccctcccccctccccccaccccacaacagtccccagagtgtgatgatccccttcctgtgtccatgtgttctcattgttcagttcccacctatgagtgagaatatgcagtgtttggttttttgttcttgcgatagtttactgagaatgatgatttccagcttcatccatgtccctacaaaggacatgaactcatcattttttatggctgcatagtattccatggtgtatatgtgccacattttcttaatccagtctattgttgttggacatttgggttggttccaagtctttgctattgtgaatagtgctgcaataaacatacgtgtgcatgtgtcttta

SEQ ID NO: 18 - DNA, 3136 bp, Homo sapiens
agaggtcacgtgacgccgcgcgggctgcgcgggcagtggtgggaaggctggcgcgaggcgtgaggtggcgtgaggcgaagctggaatctgcctctgtcacgggggctggtgcctcacgggtttgtgtcctagacagtgaagaagaagtaaaatattcacaagatgaagatttttccagaagggactttgagtcaaagatggctttttatatttgacaagtcttgtcatctgtaatgaagatcattgtgaaacagaagattgattaaagccttgtaacattggacctagattagagatttagaaaagaaagtcaaaattagtcactttagtgttagtgttcccatttcataatatttattctttcttctaaatagatttagggagtagaaattaaaattcaatgctataccaaagggtatactaatatttgtttggctttttttccctttttgtgagggagaaaaaagtagataacgaaaagctatagtcattcgtaatgaaatatactaaacagaattttatgatgtcagttcttggcattataatctacgtaactgatttaattgtggacatatgggtatctgtcagatttttccatgaaggacagtatgtttttagtgctttagcgttaagctttatgctttttggaacacttgtggctcagtgttttagttattcttggttcaaggctgatttaaagaaagcaggccaagaaagtcagcattgttttcttctacttcattgcttgcaaggaggagtttttacaaggtattggtttgccttaaaaaggggttaccatgcagcttttaaatatgacagcaatactagtaacttcgtggaagaacaaattgatctacataaagaagttatagatagagtgactgatttgagcatgctcagactatttgagacctacctggaaggctgcccacaacttattcttcaactctacattcttctggagcatggacaagcgaatttcagtcagtatgcggccatcatggtctcttgctgtgctatttcttggtcaactgttgattatcaagtagctttaagaaaatccttgcctgacaaaaagcttcttaatggattatgtcccaaaatcacatatctcttttacaagttgtttacattattatcgtggatgctgagtgttgtacttctactattcttaaatgttaagattgctttatttctgttgttatttctttggttgttaggtataatatgggcatttaaaaacaacacccagttttgtacttgtataagtatggaattcttatataggattgttgttggattcattcttatctttacattttttaatattaagggacagaataccaagtgtccaatgtcttgttattatattgttagggtactgggcactttggggatattgactgtattctgggtttgccccctcactatttttaatccagactattttatacctatcagtataactatagttcttactcttcttcttggaattctttttcttattgtttattatgggagttttcacccaaacagaagtgcagaaacaaaatgtgatgaaattgatggaaaaccagttctaagagaatgtagaatgagatatttcctaatggaataagctattcatttatgatatatattttcttatattttgtttcattggttagtaaagaaaatgtgtgttatgtgggtgtgttgtctcttatttttgccacctttaatttgaaattagttcagtgaaataggagatacatagtagtattttatttttaaaattaatttctcatttggttttgaagatcttgagtactcagatatctttctactgcctggtagagctgccatcttgagcctgaaatataagaaatggtctggttttcataatgagaaggctggaattgagcttccctcccattttccttgttcctgaactaatactactgtacctgttatggaggactgcaaagggaagagaaaagcagaacactgtattattttttcctttattgtcttcagtgcatatatttgcagttggggacaggttgagtagaggaaaagggaaagaagggaaagcagaaaacaaatttttagcatctgctgtgctttcatccatgaaatctccaattcagtaagtgcaaaagagaattggtgtgcatctgagaggtctgacatttcattatttacttatttcctagcttttctgaattaatgcactcttaacatataattatattaatcctatttgtgctagaatagttgtatctaaatcatattttaaaattatttttatttttaaaaaattatggtaaaaacatataaaatttaccatcttaatcactttgagtgtacagttcatcagtgttaactgtattcaccttgtgcaacagatctcaaggactttttcaccttgtaaaactaagattctctatttattgaacaaatccccatttcctccttccccaagtctctctcaactgaaattataattttttgtttctatgagtttgaatactttagataccttgttgccatggtttgaatgtgccccccagatttcatgtgtgtgaaacttaatctccaaatttgtatgttgatggcatttggaagtggtggggactttgtttatttatttatttttaattttttaattttatattattattattattattatactttaaggtttagggtacatgtgcacaatgtgcaggttagttacatatgtatacatgtgccatgctggtgtgctgcacccattaactcgtcatttatcattaggtatatctcctaaagctatccctcccccctccccccaccccacaacagtccccagagtgtgatgatccccttcctgtgtccatgtgttctcattgttcagttcccacctatgagtgagaatatgcagtgtttggttttttgttcttgcgatagtttactgagaatgatgatttccagcttcatccatgtccctacaaaggacatgaactcatcattttttatggctgcatagtattccatggtgtatatgtgccacattttcttaatccagtctattgttgttggacatttgggttggttccaagtctttgctattgtgaatagtgctgcaataaacatacgtgtgcatgtgtcttta

SEQ ID NO: 19 - protein, 241 aa, Homo sapiens
MLRLFETYLEGCPQLILQLYILLEHGQANFSQYAAIMVSCCAISWSTVDYQVALRKSLPDKKLLNGLCPKITYLFYKLFTLLSWMLSVVLLLFLNVKIALFLLLFLWLLGIIWAFKNNTQFCTCISMEFLYRIVVGFILIFTFFNIKGQNTKCPMSCYYIVRVLGTLGILTVFWVCPLTIFNPDYFIPISITIVLTLLLGILFLIVYYGSFHPNRSAETKCDEIDGKPVLRECRMRYFLME

SEQ ID NO: 20 - protein, 373 aa, Homo sapiens
MKYTKQNFMMSVLGIIIYVTDLIVDIWVSVRFFHEGQYVFSALALSFMLFGTLVAQCFSYSWFKADLKKAGQESQHCFLLLHCLQGGVFTRYWFALKRGYHAAFKYDSNTSNFVEEQIDLHKEVIDRVTDLSMLRLFETYLEGCPQLILQLYILLEHGQANFSQYAAIMVSCCAISWSTVDYQVALRKSLPDKKLLNGLCPKITYLFYKLFTLLSWMLSVVLLLFLNVKIALFLLLFLWLLGIIWAFKNNTQFCTCISMEFLYRIVVGFILIFTFFNIKGQNTKCPMSCYYIVRVLGTLGILTVFWVCPLTIFNPDYFIPISITIVLTLLLGILFLIVYYGSFHPNRSAETKCDEIDGKPVLRECRMRYFLME

SEQ ID NO: 21 - DNA, 1926 bp, Mus musculus
gatcctaaagagttagacagtgaagaaatagaactcataagctgaagatttccaagaagagacattgagttaaagaaggcttttatatttgtcacaaacattgttatctgtaatgaagatcacagcagaggcgaagatacagcaaggccttcttgtaccacttgatctggcgtagacatttttttttaaaggaagttaaagttattcacttttgttttagtgttccaatttcataatatttatttatttatttttcgtactaggcactgaatataggagtgtatgaatgttagataaacactccatcactgaactatatcaccatattcttttcactagttagactcagtgtataaattacaattcaatgctaacccaaaagatacactagtatccattgtggcattttcccctatttttgtatctgaaaaggagtaactaggcaatagccacagtccttcataatgaaatataccaagtgtaattttatgatgtccgttttgggcattataatctatgtaactgatttagttgcagacattgtcctatctgttaggtacttccatgatggacaatatgttcttggtgttttaaccttgagctttgtgctttgtggaacactcatagtccattgttttagctactcatggttgaaggctgacttagagaaagcaggacaagaaaatgaacgttattttcttctacttcattgcttgcaaggaggagttttcacaaggtattggtttgccttgagaacgggttaccatgtggttttcaaacacagcgacaggaagagtaattttatggaggagcaaacggatcctcacaaagaagcaatagacatggccaccgacttgagcatgctcaggctgtttgagacctacctggaaggctgcccgcaactcattctccagctctatgcctttctggagtgtggccaggcaaatttaagtcagtgcatggtcatcatggtttcctgctgtgctatttcttggtcaactgttgactatcaaatagctttaagaaaatcattgcccgataaaaatcttctccgaggactctggcccaaactcatgtatctcttttacaagttgcttaccttgttatcctggatgctgagtgttgtacttctgctgttcgtagatgtgagggttgctttgcttctgctattatttctttggatcacaggcttcatatgggcatttataaaccatactcagttttgtaattctgtaagtatggagttcttatataggattgtggttggattcatccttgtgtttacattttttaatatcaaggggcagaataccaaatgcccaatgtcttgttattatactgtaagagtgctaggcaccctgggaatcttgactgtattctggatctaccctctttctatctttaactctgactattttatccctattagtgccaccatagttcttgctcttctccttgggattatttttcttggtgtttattatggaaattttcacccaaatagaaatgtagaaccacaacttgatgaaactgatggaaaagcacctcagagagattgtagaataagatattttctaatggactaacttgtgaattcatgagaaatattttattttttttgtttcattgcctagtaaaaaaaatgtctgtcatatgtatgtgttgttacttagtttatcacctctgtctgaaatgagttatggcacatggtgaatgagagcatagtaatattttatggtttaaaataatttcttctttgtgttgctgaggatcaggcctgcacatgctatgtaaatattctaccactgagttgcacccccagccatctcgctggttccaaaagtcttgagtgttgagatagttgctttctgtctgatagagctgccatgttgttcctcaagtggaataaacaatgtggtcccataa

SEQ ID NO: 22 - protein, 373 aa, Mus musculus
MKYTKCNFMMSVLGIIIYVTDLVADIVLSVRYFHDGQYVLGVLTLSFVLCGTLIVHCFSYSWLKADLEKAGQENERYFLLLHCLQGGVFTRYWFALRTGYHVVFKHSDRKSNFMEEQTDPHKEAIDMATDLSMLRLFETYLEGCPQLILQLYAFLECGQANLSQCMVIMVSCCAISWSTVDYQIALRKSLPDKNLLRGLWPKLMYLFYKLLTLLSWMLSVVLLLFVDVRVALLLLLFLWITGFIWAFINHTQFCNSVSMEFLYRIVVGFILVFTFFNIKGQNTKCPMSCYYTVRVLGTLGILTVFWIYPLSIFNSDYFIPISATIVLALLLGIIFLGVYYGNFHPNRNVEPQLDETDGKAPQRDCRIRYFLMD

SEQ ID NO: 23 - DNA, 1909 bp, Rattus norvegicus
gatcctaaagtgttcgacagtgaagaaataaaactcatatgctgacgacttccaagaagggacattgaattaaagaaggcttttttatatttgtcacaaacattggtatccgtaatgaagattgtgatggaggagaagatacagcagggcctccttgtgctactgggtctggagtagagattttttaaaaaagaaagttaaagttattcatttttgttttagtgctccgatttcatagtatttatttatttatttatttttggtactagggactgaatataggaatttataaatgttagataaacactctgtcactgaactatatcaccatattcttttctctgagtagactcagagagtagaaattacaattcagtgctaacacaaaagatacactagtatccattgtggcatttcccctgtttttgtatctgaaaaagagtagctaggcaagagccacaggccttcataatgaaatacaccatatgcaattttatgatgtcagttttgggcattataatctatgtaactgatttagttgcggacattgtcctaactgttaggtacttctatgacggacaatatgtttttggtgttttaaccttgagctttgtgctttgtggaacactcatagtccattgttttagctactcatggttgaaggacgacttaaagaaagcaggaggagaaaatgaacattattttcttctgcttcattgcttgcaaggaggagttttcacaaggtattggtttgtcctgagaacaggttaccatgtggttttcaaacacagccacaggacaagtaattttatggaggaacaaacagatcctcacaaagaagcaatagacatggccaccgacttgagcatgctcagactgtttgagacctacctggagggctgcccacaactcatccttcagctctatgcctttctggagcgtggccaggcaaattttagtcaatacatggtcatcatggtttcctgctgtgctatttcttggtcaactgtcgactatcaaatagctttaagaaaatcattgcctgataaaaatctcctcagaggattctggcccaagctcacgtatctcttctacaagttgtttaccttgttatcctggatgctgagtgttgtacttctgctctttgtggatgtgaggactgttctgcttctgctcttatttctgtggactgtaggcttcatatgggcatttataaatcacactcagttttgcaattctctaagtatggagttcttatacaggctggtggttggattcatccttgtgttcacgttttttaatatcaaggggcagaataccaaatgtccaatgtcttgctattacactgtaagggtgcttggcaccctgggaatcttgactgtgttctggatttaccctctctctatttttaactctgactattttatccctatcagtgccaccatcgttctctctcttctatttgggattatttttcttggtgtgtattatggaacttatcacccaaatataaatgcagggacacaacacgacgaacctgatggaaaagcacctcagagagattgtagaataagatattttctaatggactaagttgtgaatttatgagaaatgtcttttttttttcattgcctagtaaagaaaatgtctgtcatatgtacatgctgttacttagtttgtcacttctgacttgaaatgagttatggtacatggtgaatgagaagataatattttaaggattaaaataatttcttctttgtgttgccaaggattaggccctgtgcatgttatcccaccactgagttgcaaccccagccatctcgctggtttcaaaagtcttgagtattgaggtagttactattccatcaagcgaataaacagtgaggcccataaaaaaaaaaaaaaaaaa

SEQ ID NO: 24 - protein, 373 aa, Rattus norvegicus
MKYTICNFMMSVLGIIIYVTDLVADIVLTVRYFYDGQYVFGVLTLSFVLCGTLIVHCFSYSWLKDDLKKAGGENEHYFLLLHCLQGGVFTRYWFVLRTGYHVVFKHSHRTSNFMEEQTDPHKEAIDMATDLSMLRLFETYLEGCPQLILQLYAFLERGQANFSQYMVIMVSCCAISWSTVDYQIALRKSLPDKNLLRGFWPKLTYLFYKLFTLLSWMLSVVLLLFVDVRTVLLLLLFLWTVGFIWAFINHTQFCNSLSMEFLYRLVVGFILVFTFFNIKGQNTKCPMSCYYTVRVLGTLGILTVFWIYPLSIFNSDYFIPISATIVLSLLFGIIFLGVYYGTYHPNINAGTQHDEPDGKAPQRDCRIRYFLMD

SEQ ID NO: 25 - DNA, 20241 bp, Homo sapiens
atcctctccctcggagtcagctggtggaggagaggaagcgggaggagggagcgcgcgcgaggggaggagaggaatgtgcaggtccgaggagcgccgcggcggccgctgctgctcctgctgctggcggcggcggcggctcgggcggcagcagcgaagccgggacggcgaggagcgcgggcggcgggcaggggcgcgcgcggggcgccgcgagcagcttggctccgcgcaggcagccaggcggcgctcctgccggccccaggcgcgccgctagcccggcccagcgcccagcccggcgggcggcgggcggcggcggacggcaggcgagccgacgcaggagcaggaggagggggagccgcaccgcctgggagggaagccggggcgaggcgaggaggtggcgggaggaggagacagcggggaaaggtgtcagataaaggagggctctcctccggtgtggaggcatcatggccgctaaatcagacgggaggctgaaaatgaagaaaagcagcgacgtggcgttcaccccgctgcagaactcggaccactcgggctcggtgcagggattggctccaggcttgccgtcggggtcgggagccgaggacgaggaggcggccgggggcggctgctgcccggacggcggcggctgctcgcgctgctgctgctgctgcgccgggagtggcggctccgcgggctcgggcggctccggcggcgtcgccggcccgggcggcggcggggcgggctcggctgcgctgtgcctgcgcctgggcagggagcagcggcgctactcactgtgggactgcctctggatcctggccgccgtggccgtgtacttcgcggacgtgggcacagacgtctggctcgccgtggactactacctgcgcggccagcgctggtggttcgggctcacgctcttcttcgtggtgctcggctctctgtcggtgcaagtgttcagcttccgctggtttgtgcacgatttcagcaccgaggacagcgccacggccgctgctgcctccagctgcccgcagcctggagccgattgcaagacggtggtcggcggtgggtctgcagccggggaaggcgaggctcgtccttccacgccgcaaaggcaagcatctaacgccagcaagagcaacatcgccgcggccaacagcggcagcaacagcagcggggctacccgggccagtggcaagcacaggtctgcgtcctgctccttctgcatctggctcctgcagtcactcatccacatcttgcagctcgggcaaatctggagatatttccacacaatatacttaggtattcgaagccgacagagtggggagaatgacagatggaggttttactggaaaatggtatatgagtatgcggatgtgagtatgctgcatttgctagccacctttctggaaagtgctccacagctggtcctgcagctctgcattatcgtacagactcatagcttacaggccctccaaggtttcacagcggcagcttccctcgtgtccctggcctgggccttggcctcctaccagaaggccctccgggactctcgagatgacaagaagcccatcagctacatggccgtcatcatccagttctgctggcacttcttcaccatcgccgccagggtcatcacgtttgccctctttgcctcggttttccagctgtactttgggatcttcatcgtccttcactggtgcatcatgaccttctggatcgtccactgtgagacagaattctgtatcaccaaatgggaagagattgtgttcgacatggtggtggggattatctatatcttcagttggttcaatgtcaaggaaggcaggacacgctgcaggctattcatttactattttgtgatccttttggaaaatacagccttgagtgccctctggtacctctacaaggctccccagattgcagacgcatttgccattccagcgctgtgtgtggtgttcagcagctttttaactggcgttgtttttatgctgatgtattatgccttctttcatcccaatggacccagattcgggcagtcaccaagttgtgcttgtgaggacccagccgctgccttcactttgcccccagacgtggccacaagcaccctacggtccatctccaacaaccgcagtgttgtcagcgaccgcgatcagaaattcgcagagcgggatgggtgtgtacctgtctttcaagtgaggcccactgccccatccaccccatcatctcgcccaccacggattgaagaatcagtcattaaaattgacttgttcaggaataggtacccagcatgggagagacatgttttggaccgaagcctccgaaaggctattttagcttttgaatgttccccatctcctccaaggctgcagtacaaagatgatgcccttattcaggagcggttggagtacgaaaccactttataaagcaaaaggagttgcaggacccacaacatccagatgaaggggtgacagcagggctgtggccataatgacacttcatcctagagcagggcagtgagccgtgaagttcctagtgggaccgtcatcaccattatcatttgatcctgtcggctgggggcggctggtctccttccaaagcagctgcacccgagagtctctgactccacctgaaagaatgacgctggcttaataggactctccattgctaccaaactcctcctgcacggtcttgggtgcacccaccagagggtactactattatggaaaaattttgcctccaatcattagggtgtcttgatggcgttaactgatctttccataaaaatagattcagtcatacacacatacacacactaacacacataagttacaccagtcctctgtcaaaaaagcttaggtgacttttcttgatgcaaagctctgattcccacaggaatataaaaacaaagaaagagggaaacatccctcgagaaaaaaaatagtattgcttagaaaagaaaccattttctcatttggaaatccataccatgtgtgaaaatcctatccaacggacagcaaacccaaatgttgtctacacatgtgttagcattgatggagtggttcattttctacacatttcaggatttgttttatattttaaattttcagttgcgaacatcctttttgacagaaatcctatgcagcccatgtacggctttcaacaagaccaaggagctcaataacttcatgatgtaaattaaatagtaatcatgattcagtattcaattgcaaaaatgtaacaggtacacaaagaggaagtggggaaaaaggcaaaatgagagtctgattcccaggcatgtgcagcgcccattgggacataacggcagtgcggcgcgagccagaggaatgggctggaaccggatctgtttccagacgcagaatgagtggctctgtgtgaccataggcagatgctgactctggaagactccgtgccactcctttctagtgccaaacaccatccaaccacaggactgacgtggaagccccaaacaactgagaatgagtggcatgagccccctaaaagcaggcgagagaacgagcaatcaagttctccactgtgtacagacttttcctccccccaatccaaggtcaaagtgatgtgtcttttagaggctttgggacactttttagtaagtatgagcagacaaatgcaatgaatatgctatgaaaaaacccttctgaactgagagagggcttatcactatatccagctaagatttgtatttgaatcatctgtaaagtcgcactcttacaacaagcttctgggttttaaatacctccgtacagcaagtaaacgttccccgctttctgttctcagtgtcctcggtcatggtgcttttcgttgcattaaaagtgccggtcaaactttgatagtatttttttatagttggtgcagagtggaataactcatggattatttcaatatttttgtaataaaaaatatagggtatacacataggcatcatcacattttttatagacctggaatcgtttaaaatactttaagcatcataattacttgggatgtcagaaactggtccacaaattccatcagcctgcctcagcagattgaaaacatttgtctcttgcaagatcaccctactttgcaagttggtgcccccaggaacctggccaggggtgctatcagaatatcaggtgaagagagaatcagcttaaatagaaagggcttgtcaagactggccaatgtttcccaggaaatcaaagatgtaaatgattactttcatccatccattataacaaacctgaccacagtggaagctgtcttaaacttccttccctggttttatattaacccaactgatagattaagtattagtcaaaccactaaaaaagaaaaagaaaaaagtttaacttaattattcggttatttggatctaattcacacaaagtagtccagttctctagccaccacctgtaatgggtgtgtcatccagagactgtgtccccacgatgacatccacaggaagtaacagagggctcaacctaggacttcttttggtacaaagccccaaatcaatttttttaaaaaatagacaatttttataagtagacatacttcctagtactccatgatttgatcctccaagcaagatttccactaaaaaatactaatcttttgttgggatgtggaaagattacctagtcaccagtaaaggcccaggaaaaggctcttcttgtcagcacatggtgaaaacattccatccccactggagaaggaaaaaacgattttggcaaattcttcacttttgtgcagaaccttgagttattagcttcattgtttccaagacaacttttaactgatgatctttggaaattgagtttctcagttgaactgtacctttgattctatgagtaaatcacagattacagtctaatagagtcaatcaatcaacacaaacccaacaggccccatcatgcttcaatcatgtaagttctaagttatttctcaacttgatccctcattcaacatgttaagagtcagaatgaatactatgtcaatgaaaaatgatgtactgtgctttgacttggaggtgagattggcagtcaggagaatgtaaggaggttgaatttttcagtgatttcccaaatactgtaaatactctgttatccgacatatttggagattatgatgttttaattaggcatgaattcttgttaaggaaagaacatatccatgatgtgatgaattacaacctttcaaaagattacaagagcaaaacaagagataaatcatgatttagccttgcttccatgattcaggaagcactacactgccatcagactgttgtggtaataacaacttttacttgttttctagatgcacagataacagagagtttaaagtattcagatttaaagagacatcatcagtgtacaaagaaacaaagtttcatttttgtatttatattttaattctaacatttccttttcaatctgccattaaaccctccgcagacagtaactggagaatcccaaaggaaaaaattggaaatgctgggttccttatctgcaggctcctttctgtgtctgagtccactttgattccatttaagagggagatctgctcttactcactttttgcataggatcaggaaattttctaaaggaacaacattgtaatttgttttacttttaaacttgcatttctaaatatgaaaccatgtttaatgaatatatataatgtgtgtgtgtgtatcttaaccatagtgacactttaagtgtttgtgtgaaagaaaaggaaataatttttccatgtaagtcaaagtttagtctcccaaaatgactatgtcctttaaatcctctttgcttatttacttaactacatactgtctagttcaatagcactgactttgcagacacttagttactactcatttgtgataaacgctgttaacccaacaaatataataaattctcttactgacatggcaagaatatataattcaagtattagcaaaagataatctgaggataaaagtaaaatgaagtattttatggttaatttctaaatgcccaatttattttgctctatgagtaaaggaagtgattgcacagaacaattaaaagtgaatgagaatagttgaaaactcaatggctgttttttaaaaatgatatgtgccttttaagtgtgtttgtgtacatacatatatgtatatatacgtacctatatatgtatgtacacacacacacacacacactttccaactaaagtaacagagatgaaaaggataaagtatatactgcttttgaatgtatataaagtggtatgttatgcatataaattgtacataaactttttagaaaagaagcattttcctgctcctttttcaaaaccaacccaagcttacagtccatctataagaccaacacacttacgaacttcagttggaaatacctaaatataattcagcacttcttagctcgaatgagttttatcacttcttaaggatctcatcttttaaacagctgaataaaatagttctgtgtcacttcaaagtttctttctctgaacagattgaattgagcaaagagaacctcttctgtccttaccaggattgtgtaaggttacacatttgcttttaaatataccaaatgccgttgattggaaacaagttctgacacaatgtttagacaagaatccagagattttttctaatgaaccattttctagactaaatatatgctcccttgcattttccacatatctttgccattagccattgctgtttctatataaagcttggatgagatgcctgcatttttatgtgctaaggagaattccttaaagcctttttaaaaatagctcatactgtcattcagattatagctcagaggatggttgaagcgcatggtgaaaacacaggaggactggggtggtcattcctataatttcagtgacagatgcagatcaacgttcctttgtctcggcaatccaatgtcatttttgaaaacaatcaaaaagatcgcttgtgtcagcttctgactcataacactcctcccacctgatgctccagtgtttcaaaatggccaaggatgggcgattccgctctatcccccatttctgagactcttgtctggacctgtaacaggccgtgaaatgccctgagcattcgagtggcatcccttctcctcacataggcacctgggtggcagcatcagaccactgaagttgttgtgttgacatatgtcttatctagttgctgtcctaaaaatgggcatgtggcaagactctcaatctacagcctcgacagtatcattactcattctaaagtaaaactgcagaatatgggtggaattgtataaaaacataatgagccatttaattttgctaattgaagcaattagtctaacatgcaagcagcctgctctcacagcagagagccacatggaagaagtgccaaatagccatttgcatttatatatatatattgcaggcagtgacctggcccccaaatgtaaagcttttgtcaaccttgaggcctatattctgctaaacaagagatgacttaatgtccttgaaatattttcgtaatatactgacagcctaatgtcagaaacgagctgcctaaatcaagttttgcttttggttatttcacttccccatagactttcttatggttccatctcccacattgagagtagctcaccacgatggatggtttactgcgcacctagtgctggactaagagctgtatctatgtggtttcatttagtcctcactgccatctgtgagttaagcatcatttacagatgacaaaatctgtaaatggcttagagatgtcaagcaatttgcccaaaggtcccacagctaggaaacagtggggctgagggttgagcacagctttcaacaactgcgacttctgggagcccagtgactcttcccacaaaatctagtcctgatttggcaagtcttcagaagaaacagaatcatggtctgatgatcaaatttttccaagaaaattttatttaaaagtcaaagatgtccttcaaaatgaacagttaaaaatgtaaaagtcgatgtaaaatggaagtctctatcacctgtaactaaattttaccttaactctaactcatagtaggcagataaatgctattcttccattccaggcaactgtccccctcctatggctccactatgtattcaattaagtgataaatataaattaacctgatgccatgtctcttgtattttatatgtgtatgctgttttcatccaattaagcagactgaaaaaaaactaaaccccattacttactttggcattttgacaagatagagagagaggaaaagaaagagggagggagagagggagggaaggaagaaggaaggaaggaaggaaggaaggaaggaaggaaggaaggaaggaaggaaggaaggaaggaaggagatttaacaagtctttgaagtgatattttcaaattataaggtaattctgtttcactgccataatttttccctaaattttatttaatatcttgcaggtcacaaactttaatatttaagaggattattaaaccactagcttgaacaatcatataagtctaggaaccttattttagtgttagatgccaataatactgcaagtgtcaaccaaatatttgttgaattgaattataaaataattgatgtgttctttcccttctcactttagatatagcatgtctgaaggtctgcaagatgacagagttgtaacccattcaatgatattgttgcctagtaagctgtgtgtgtgttgtttgaactgatactaaaaaggtagctgataataaaccaaaaattttctcaaccctggtgtttatttttaaaaaatcttcaatgatcaatatgaatgtagtgtattaaaatacaagtaactatcttcctactttgatttaagagatctttatgaatttatataaaattagaagtcactgatttttataggaaatagcatgtaaaataaatctaagtattgctttatcactttattttatagatgagacaactgagatccaaaaagaacaggtaatttttgtgatcaggattacacaatacacttttttttttccctgagtcatttattcaacaagtttgacctctacaactcatttggctaggcaatgcacagtcaagcacaaaaggaaagttgcactggaatagctcatagtctggctattagcagcacaatcatagttttctgacgccagctcttactcttttctactctaccacactgtttcttctcttctcaatatctatatttaattccatattgaagcaagaaagaaacacagcttttctaagactatgcagtcatgtgtcacttaaggatggggatatgttctgagatatgcatcgtcaggcaattttgtcattgtgtgatggagtgtgcttacacaagcttagatggtagagcctaccatgctcctaggctatatggtagagcctattgtccctaggctacaaacctgtacagcatgctactgtaccgaatactgtaggcaactgtaacaccatggtaagtacttgtgtatttaaatatagaaaagttaacagtaaaaaatatagtattattgtcttatgggatcgctgtcatatgtgcagtctattattgaccaaaatgccattgtgtggcatgtgagccttacaatatacaattaacatatgaaataatgatgatgaacataaagtaacaatacaaatacaaaaaaaaaactagatgactgcttataaagagaaaagtaattttataatttgtttatatgactctccaacactagatatttttaaattgatatcacaacacacaaaaaaattgaaatactctcttggtgcatagtatttgattgaaaacaatcatttttggataaactttgaagcgattcttgagaacttatttcaagaaaaggcatgaaattagggagactccaaagtgaagagttttccaataggtgacttctctgatttttcaagaaagcattcttcactaactgtatttctccagcatactggttatttaggaataacaaatttctggacataaacatgagctgtttctctaaagcctttcctccaatgcccagaagagcagcactgtgctgcgtgacaatttcaggagtcaggagtcaggagtcaggacagtcagccccagcttcctggggaaacccacactggctttggacccgattgcattctctcctgagtgattggcttcccacatatataagcagcagattgttaaagatcactattaacttgtataactaattttccttatgtgaaataattctggtcagggaatatataaacccattggccctctaaggagtagaagaaaagagagaagaaagtatattaacttttatgagtacagaataattcaagttccttagcgagtcacattatgcattaataaaagagttgacctaataaatgttacaaggtaccatgatctctaggttcatgccaccattaccacattccttactacaattattgctattttagtcattggaccagacaaaatgaagcatataattactgatataatatttgctaagcaaaaatcttgtttaacgaaaaaaatcaataccaaaactaattaatcaaaatattaagcaaatattaccagcacagtactgacacaaaattttctcttgtgctagtaattgaagtatgtcatctaccctgttattagaatttcagaaaataggccgggcgcagtggctcacgcctgtaatcccaacactttgggaggctgaggcgggcggatcacaaggtcaggagatcgagaccatcctggctaacacagtgaaacccccatctctactaaaactacaaaaaaattagccaggcatggtggcgggcgcctgtggtcccagctactcgggaggctgaggcaggagaatggcatgaacccaggaggcagagcttgcagtgagccaagatcgtgccactgcactccagcctgggtgacagagcaagactccgtctcaaaaaaaaaaaaaaaaaaaaaaaagaatttcagaaaatataaagttttatgtttttattatatttccatctaccaaattgttgaccttctcctcctctccattgcttaatttatattaaaacagatttaatcaaattattacttaagtactacaaatgttatcagatggagatgtggttaagctaatttaatttacctattctagtggcattctggtatggagctgtatcaaatcaacacttttaattatttcacattaattcatcaagaagttccaaaacactactaaatgtgttgaaaatatagtttgagtttctatgattgtaatcaaaattcctattttgatcgcacaccagtagaacgcatcttaacaccagcattgccattgtgagtctagaaaatgagcactttgtgtgttgagcgctgttgcattcacttagcaattaacctttgacctgtggttttctgctgagccccttgtgattttttttattctattcaaattgggagcaataacacaccttaacataaccaaaaaaaggagacctgtcagctagtgaaagaattgtcattttatatcattctttcaaaaaattaaaatattcaacttcccttattaacctttctaatgcattgtacataaaagaggaaatggatttctgaaatatattttgaaagcctggggtgaaacattttccacggtctgaatcggaagcttggggctctgtggaaagatgtaaatccctcctgctgtaagaggagggaaggcagcagtgagctgtcactcagaaatacagtcaccactgtcacaaagctgcctattgctgatgctatcgattcccttctttttctacagaaacatcttggagcttgtcaagctttactggaggtgatttgcagttaattaattcaacagacactttaatcttgcaaattcttgacttgtaatattgtaaccaagctcctgcaagggaacattaatcagttagtgaaaaaggagcacttccgttcagccgtagtaccatgacgtgcacaggcctgaagagaaatacctctgtgaagtggagcgctagtgaattcctgctacctgcttcttatggctcacgctatgaatattcacctgcttcatttgttttttccagtaaacgctgttttgaaaaaaaagaaaaatattcccgggggcttgcatagctcagagaacggagtactgggtcgtggagacttgctttaaatggattcaaatccacatgtttggaaatgaaaataatgcactgtcatctgttgaataattgatctgtctgagtacagttgctgcttttatttcatttcttgagactaccattgtcagcattgtaataaccaatttataaaaattgagtttttattcagtttcagaggtaaaatctgcatgggtgcagctactgaataatttgattcctgccttcttaggtggtgacattagcagttccaaaccgagatccatttctatgtggaattggctatcctgttgcttctcaggccctgcaaaaccttggttacgagctcaaagatcacgaatctgatattcttttttttttttttttttttttttttttgagacagagtctcgctctgtcgcaggggctggagtgcagtggcacaatctcggctcactgcaagctctgcctcccaggttcacaccatccttctgcctcagccttctgagtaggtgggactacaggcgcctgtcaccacgcccggctaatttttttgtattttttagtagagatggggtttcaccgtgttagccagaatggtctcgatctcctgacctcgtgatctgccctccttggcctcccaaagtgctgggattacaggcgtgagccaccacacccggccccgatattcttaatgactaaattttcacatagaggtaaacagatcatctcttaatttaatacatggttctttctcccttgcttctgggttttgttttttttttttcaaagaaagatttgagctacgagataagaatgaagttaccagaagttatcaggtcatagtttcagagtatgcaagagagtcgggccttcatatgttcttgtaaagttttctgtctaatcttttggtataacaattttaggagttcaccctagatgaaagagtggaagtcatcagatttgtcaataagcagtctagaggaaaaatgagaagaggaagaagcagggattctttttcttgtgttttgaagatgtttctcctcccaaagctatcaccttggtagttatcaccaagatgtataatagcaagcactactgaatgatcttcccagttatcagcactagcatcacggcgagtcagttttcagaactagctcttggcgcaagccctgaaataaaatggggacaaaaagtggtctaccaccatgtgacttattttctttttttttttaattttattattattatactttaagttttagggtacatgtgcacaacgtgcaggtttgttacatatgtatacatgtgccatgttggtgtgctgtacctattaactcgtcatttagcatcaggtatatctcctaatgctatccctcccccctccccccaccccacaacactccccggtgtgtgatgttccccttcctgtgcacgtgacttattttcaattgcccagcaatgaaaactaacaagttaaagaaaatgttcattttctgaaccccagagcccacataggtacaaagatactctgtaatgtacaatgaggtggccaatcgtgggaatataggagcaataaatagtcctcttaagcaaggttcatgggtaagagttactctagcaggattgggtgttgggtcagagggtatctattaatgtagaggcccaagtatggtgatgaagagaaaacctgtcagtggctcatccatagtatttgccttttcacagagcagagaagttcaaaatagtcacagccagtccataactataacaacagacatgtccactttggaaaggctagggcctgacgaaagtgggaaaacagagatgtcagtggtgtcatgtctaagagtgactctgtcattaggggaacccaccccctgtgatagttctccttgaccactggtccctatgggctctgcaggagagcttctcgtgggttctaagataaggtattccaaggtattgtaagttacccttgtttgtagaacatgaaccacttaaccatccctccttttaacagcaatgagattcagggttaccatggccttactcatcttcccattgtaaatatatcacaatgtcacaagagcctctgtgtccaaacacactaaactgggtttacaagcattagaatctttcactcatattgtgaatctcaattctgccagtcacctagtctgtgtatctgttcccaaactggaaaaaataattcttgagagaataattttcagaataatggaggtggaaagaaatgaacagttaagcaatttttcaacatagacaaaaccactggaccattgatagccctcaagctctgattcttcctcctgactaagtttcttttctttggggggctttcaacatctgaattttccagatgattgcggaaccatcgtcactaaaccaaagtagacaaggagttattaaaaaataaagactgtccacatgactgcaaatatcctgatgaaaagtggccaagtagatcactcaagtggtaaatttggtcttcatgatatcaaacatacggatatttggaaaagtcgagatgtttgaatcatacagttttccgtctgggtgtctggtgtttctggatagacagactgctccggtgttgtaagtaatggaattgaactttcttgcgccgtaagcaattgctggtcatattctgctgctaaaagtctctttgttgtgccaagagaaataatgcagaacaaatgttatttaatttttatttactttcagcaaacacatgaatgaaagaggtcaggtaggctgtcctgggcattctgggcctggctgcggcacaccctccttcacttggcccctgccaggcaagaaactttctattcagtctttgctatctttcataaattgtatcattgctcttctgctgttcatatcatcttagttattcacaaagtctacttgataaaatggctcaagggaaatacaagtttcttaagtttttattcttcaaatagaagttttaattttaagcattccttatgatattttttaagcctaaaaaccattcaaattgcttgacaaaattatttcatggtgaattttataaggttgatagaagtaaaagctatttttcccaaaacaaacaaaataccatacatagttttttgggtttggtttgttgatgtcatgccaatttccaagcaccaactggttaccacaaacatgggaatatttagtgatatctttgtagtcatcgttaaaattcctgggaaaaaaagaaaaagtttacgtcaaaggaaaattcacctcccacaaggaaagtctgagatgttcatcctgacatttgcgttcctgattatttgtggacatttcttcattgtgactgtaggaagctgagcttgtttctcctaatttgacactgggttggtgagcattgtctcaaattttgtgcttgcctcatttatggtcctgaagcttagcagaaaaacagacaagctattcagaccagttttctttaagagcacttatgttgcagaacatgatacaaatgattcaccgtgagcaggcacacagagtacggaaaggtattcaactatgcaaagatattgaggggatttccagagaaaacttaaatgttttgaagatttgtaggtagggttttgattgtgtcacattctacactcagtgccaagttagaatgtctttatggggaaggcaataaagttacttgttgggtccttccttcccttacaaacagaatgtttttatgaaatcaaatggatcctccactttgtgtagtaaggaccccccaggccccacaacatcatcactgtgagtcctatcgcagatgtgtgtaccagcccaattcagttttgcttttctttttccctaagatttttacttcaccaaatcccatttcaaatctttttaccttcatgttaccaacaggatgtttagttgaatcagcaacaaagacgtgacaacctattgtcctccacaaaagcatgagtcattttattcagtgatctttggtagtacgataatcaatggaatttatggtgtcgtagaaaaccaaaaatccatgttgaatatagtgactgtcttaaatatacttaaatatgttattctacaaaacaatatccttttacactatgggatggattcctttctggatgcagggatgggagggtctatgggtcagtgactgggacaaaggaactgggaatctctgcacaactgagccctaatccctggtccatctctccagcctcagaaactcaccctcagcctcattttccccatatgcaaaagagagatatttatttacctacctcataggggtgttgtggagattagctagatttgctaaagtgcttgtaggttagaaagtgctgtcattcctgagaactggcattaacagaagagagctgtgtgcagcacggaggaagtggagtctgaggaatacaacagcaacaactcaccaagcagagaatacaatggttcttcatcactatataaaactaacacttttccttcaaaggtctatgtataattttcttcaatgattagctttttaatgagacaactcctttcatccagacattcagatgctttatataagttggcaattttcctgttaaccaaactgaattttattaaatgtttattaaaatgcacccagaaaacttgtctcctcctgatgcctgaggggtttgcatgcctgatcccaagctgcattttttcagaatgcgtgcatgatgccccagttctgtactcatgatcaccaggtggcgttctgaaatccactactggggaaagatttttaacagatattagtgagattagagttggtgtcatttccattgagtatcctcttcacccctaagatgacacatctttacaacacaataaaagaacgtaaagccttatttccacctgtaactcctgaattgattcattttcacgttataactacatttcaaatatttcggagaagtttttacacagggcttcagctatatactgatatacatatgcttacatgtgcttaggtgggaattctactaaaggataaaggacacagtgtgaaaacaacatcagagaatatcctgtacaacttccccaaaagtgacaagttttcttgtacttaaaaatttaatcctgataagaactaatgtgaaataacatcattttggtttataaatatttgtaatttttgagacatagaggcaatatcatgatataggaatacattcataaaactagactagcaaagcagataatgttttcatgatatggcttcatgaggcaaagttgttgtacatcaatattatcattgtgcccttatttaaggattatattccattgtgaaaaaaatgtgcacactcttaaaaacacaaaatgggtttcagaaagtttaccttgagaagtgggtttgaaatcatcttgtgcttggagctgacataagatacgcactcaatataatctcttctggattctaaaatctaattggcagtgatatttcaaagccttaacatttcaaggtggttaattaatatctaatgcatggtattaaactttctgaagcatgaatttaacctaggcaattatctgattcattttttttaaagtttgtgctgccaaaccagactgaagtattgtttttctggactaatactatgagaatagtaagtgagtcaaaaaataattgggactgtcctttttcccctgcccacttcctagtatcaatactcccccaaccagaaatgcagcagaatatcctttttgctataaaggaaaatactgtgtttttatttgtttttgcagaagaaaactggtgttgcctatttggactagatgtaggggcctggaagaaggaagtggcagattcacaggtggggtgaccaggatgggaggaaaatagtggggcgagtatgtcatggggagattttgccacaaagatacaaaacagaattgaagtgtgttagagctggacaaccctttgaaatgacagagtctagattcttcaccaaacagatgaaaagacaagtagagacaacatgtacttgagatataagctatacatctcatcactggaagaaaggagacttcagcctcttttcaaggctttccagaccacatggaactctccagagccctccttgaaagtttttagaaaaactaccattttcagcaaagattcatgtgattatgctgctgaggaccagtcattctgtaaacatcacatatgtgatgctttgtaaatgtattaattgtggtcaattttcatggatatttcccattaacattgtattccatgaacaagtgatagaaaacatatggaaattctcttttgatcaaaaggagtgtctcccaattagtttacgtgtgttagtattgctgacatattattatcatcacaaaattccttttatatctagatggtatcaaataagaaaaaaatgcatcatttggtcaattgcttattgaagatcccagctgaagcctttctttggtaaagagcgcagaaagagaccatagctattcttggatgagaaccttgcctctactaaatagtttctgcttttcctctctgtagccagacagctcaatagcctagggagagtcgatgaaggatatgcaaattacatttttcccattctcagaacaaagacagcaaccaatgagccagaggtttcttctctctttgaaaccaaatagcacgctgaatttagggctatgacaaaaatgttgttaaagcaagagcaaaatcatccttcctatggattcttttctcagtgtttacttaattctttttgcagtttggattggagtttctagtaatgataattaatgccattttacatgatagcttcaatgcagaaatggtgtgagcctgagttacaaatgacatgactagggatacaaacttcgtctgtactaacatcctaccaagcagattggaaacaaatactactaccactaatattctgatgtaattaataacatctaatagaaaaatagaaacatcgtgcttagcatgaaaccattgcacaatataaacctgctcccaaatggcaaggatttttgctaccaatatttgttcttaattctccagttattttaagtaaataagtttcacatctaactacctcagctactgttgttttatttagaaacatgaaaccatgcactttgtaatcaataagtcttttgtttaacatttcaaaaggatatttggtgcaaagcaattttcaaaaatttgtacatgatatacaccacccaacctcaggaggttgtacttaattttgtttgtttgtttctaaggttggttttgggtaaaatcctcatttccactcaacatcaagataagctgctctatatttgcttaatttgccttaaacattttgtgctcctttccctgttcaatttttttgttttgttttaaatctatctctgaaaaaaaaatggaacaggtggcaggtgaacagcaaatggaagagaatggaccagtaatttctcagtcccctgttgtcaactatctgcatgacattctgattgtgcaaaaatgccattcctgtgcttccccctccattacagaataaggtccgagagaccccacgagtgtgcgtagggaacggtgtagacatttcccccagtatgagcacagtgcctggacctgaatgatcatcttggcagttcttgtgcttttactttgtaaacattgtacaaatgtatttggaattttatttgaaatggagacttaaactagttattaaatttgtttccttcctgtaaatatatatattcaaattccatgtatccaaacatccctttagcgttcagattgtaagtgtgtctttattcgcgggaggccactgtcagcaggcagtgacccccagtgccctagtttgaagcacagtgtgtggagtatttgatgtactacagtaccatagttattttggtctgttaagtaagttgcaatttgtgatgaaatgaagtggaaagtagtacttcataatgaacaaatttccttggttacatggtttttcttgtaaaacttaaagaaaaaaaaagaaaacttgaaatttta

SEQ ID NO: 26 - protein, 650 aa, Homo sapiens
MAAKSDGRLKMKKSSDVAFTPLQNSDHSGSVQGLAPGLPSGSGAEDEEAAGGGCCPDGGGCSRCCCCCAGSGGSAGSGGSGGVAGPGGGGAGSAALCLRLGREQRRYSLWDCLWILAAVAVYFADVGTDVWLAVDYYLRGQRWWFGLTLFFVVLGSLSVQVFSFRWFVHDFSTEDSATAAAASSCPQPGADCKTVVGGGSAAGEGEARPSTPQRQASNASKSNIAAANSGSNSSGATRASGKHRSASCSFCIWLLQSLIHILQLGQIWRYFHTIYLGIRSRQSGENDRWRFYWKMVYEYADVSMLHLLATFLESAPQLVLQLCIIVQTHSLQALQGFTAAASLVSLAWALASYQKALRDSRDDKKPISYMAVIIQFCWHFFTIAARVITFALFASVFQLYFGIFIVLHWCIMTFWIVHCETEFCITKWEEIVFDMVVGIIYIFSWFNVKEGRTRCRLFIYYFVILLENTALSALWYLYKAPQIADAFAIPALCVVFSSFLTGVVFMLMYYAFFHPNGPRFGQSPSCACEDPAAAFTLPPDVATSTLRSISNNRSVVSDRDQKFAERDGCVPVFQVRPTAPSTPSSRPPRIEESVIKIDLFRNRYPAWERHVLDRSLRKAILAFECSPSPPRLQYKDDALIQERLEYETTL

SEQ ID NO: 27 - DNA, 3652 bp, Mus musculus
gcggcggcgggcgagcgggcgctggagtaggagctggggagcggcgcggccggggaaggaagccagggcgaggcgaggaggtggcgggaggaggagacagcagggacaggtgtcagataaaggagtgctctcctccgctgccgaggcatcatggccgctaagtcagacgggaggctgaagatgaagaagagcagcgacgtggcgttcaccccgctgcagaactcggacaattcgggctctgtgcaaggactggctccaggcttgccgtcggggtccggagccgaggacacggaggcggccggaggcggctgctgcccggacggcggtggctgctcgcgctgctgctgctgctgcgcggggagcggcggctcggcgggctcgggcggctcgggcggcggcggccggggcagcggggcgggctctgcggcgctgtgcctgcgcctgggcagggagcagcggcgttactcgctgtgggactgcctctggatcctggccgccgtggccgtgtacttcgcggatgtgggaacggacatctggctcgcggtggactactacctgcgtggccagcgctggtggtttgggctcaccctcttcttcgtggtgctgggctccctttctgtgcaagtgttcagcttccgctggtttgtgcatgatttcagcaccgaggacagctccacgaccaccacctccagctgccagcagcctggagcagattgcaagacggtggtcagcagtgggtctgcagccggggaaggcgaggttcgtccttccacgccgcagaggcaagcatccaacgccagcaagagcaacatcgccgccaccaacagcggcagcaacagcaacggggccacccggaccagcggcaaacacaggtctgcgtcctgctccttttgcatctggctcctgcagtcactcatccacatcttgcagcttgggcaaatctggaggtatttgcacacaatatacttaggtatccggagccggcagagtggggagagcggcaggtggcggttttactggaagatggtgtacgagtatgcagatgtgagcatgctgcatctgctagccacttttctggaaagtgctccacaattggtcctgcagctctgcattattgtacagactcacagcttacaggccctccaaggtttcacagcagcagcctcccttgtgtccttggcttgggccctagcctcctaccagaaggctcttcgggactcccgagatgacaaaaagcccatcagctacatggctgtcatcattcagttctgctggcatttcttcaccatcgctgccagggtcatcacattcgccctctttgcctcggttttccagctgtattttgggatatttattgtcctccattggtgcatcatgactttctggattgtccactgtgagacagaattctgtatcaccaaatgggaagagattgtgtttgacatggtggtgggcatcatctacatcttcagttggttcaatgtcaaggaaggcaggacacgctgcaggctgttcatttactattttgtaatccttttggaaaatacagccttgagtgcactctggtacctctacaaagctccccagattgcagatgcatttgccatccctgcattgtgcgtggttttcagcagctttttaacaggtgttgtttttatgctgatgtactatgccttctttcatcccaatgggcccagatttgggcaatcaccaagttgtgcttgtgatgatccagccactgccttctctctgcctccagaagtagccacaagcacactacggtccatctccaacaaccgcagtgttgccagtgaccgtgatcagaaatttgcagagcgggatggatgtgtacctgtgtttcaagtgagaccaactgcaccacccaccccatcatctcgaccaccacggattgaagaatcagtcattaaaattgacctgttcaggaatagatatccagcatgggagagacatgtgttagatcgaagcctgagaaaggccattttagcctttgaatgttccccatctcctccaaggctgcagtacaaggatgatgcccttattcaggagaggctggaatatgaaaccactttataaaatacaaggagccgcaatgtccacatgaaggggtaacagcagggctgtggcaataatgacaccttatccaagagtagggcagcgagctgtatgttcttagttgtggtatggtttgatcttccatcagctgactgcctgctgctggtgtctattcaagccagcagtgctgagagtctcttacactgtcagcttaatatgactgttgctacaaactcctccagcagagatttggggcacattcactggaggataacattattgtgaaaaatgttgcctctaatcattagggtattttgatgggttttactaagttttgcataaatatattcacacaccaccataccacccctcaatcaaaggagttaaggtggggatggagagatgactcattagttaagagcactgactgctcttgcaaaggacccaggcttgagtagttcactgcaactctaattccagaagatctaatgtccatttttggcctcctcaagcactgcacacacatggtgcatagacatatatgcaggcaaaatacccatacacatagcataaaaataaatctcaaagaaaaaaagcttaggtgatttccttgatgcaaagctcacaacatactccaggaagaaagcagcatacttgggacaattatataaactgttctctcctttgcaaaccagtagcatcaatgaagtggacagcaagactcaagtgtttacactcgtactaactagctttgatgggatgattctttttctacatatttcaggatttgtttttacttttaggttttgcagatgagaacattcttcatgacagaaatcctatgcagcacttatatggcttttgatgagaccaaggagctcaatatctgtaatgtaaattaaatgctaatcataattcagtattcagttgcaaaaatacaatatataaaaagagtctttggggaagggacagagtgagattcagattctcaggtgtgtgcatcttatattggaatgcacccacagagccacaggagaggaacagggactatttcaaggtctgtgttcatgtctgtttccagaactgtttccaggtgcagaatgacatgggtcagcaggtatgattccggaaaccacgtgccacatctttcgagtgccaaattttgtccaattacagaactgatatggaatccccaaaatctgagaataagtggtttcccaaaacagacaaaagaagaataatcaggttccctgctgtgtacagacttaccctcttcccatccaaggtcaaaatgatgtgtctactagagactttgggacacaatttagcaagtgagagcatacagatgcaatgtgtatgccattaaaaatactgcctggactgcttgagggcttaccactccatcagctaagatttgtatttgaatcatctgtaaattcgtgctcttacaagcttctgagttttaaatacctccacacagcaagtaaacattcccgctttctgttttcggtgtccttggtcatggtgctttttgttgcattaaaagtgccggtcaaactttaaaaaaaaaaaaaaaaaa

SEQ ID NO: 28 - protein, 647 aa, Mus musculus
MAAKSDGRLKMKKSSDVAFTPLQNSDNSGSVQGLAPGLPSGSGAEDTEAAGGGCCPDGGGCSRCCCCCAGSGGSAGSGGSGGGGRGSGAGSAALCLRLGREQRRYSLWDCLWILAAVAVYFADVGTDIWLAVDYYLRGQRWWFGLTLFFVVLGSLSVQVFSFRWFVHDFSTEDSSTTTTSSCQQPGADCKTVVSSGSAAGEGEVRPSTPQRQASNASKSNIAATNSGSNSNGATRTSGKHRSASCSFCIWLLQSLIHILQLGQIWRYLHTIYLGIRSRQSGESGRWRFYWKMVYEYADVSMLHLLATFLESAPQLVLQLCIIVQTHSLQALQGFTAAASLVSLAWALASYQKALRDSRDDKKPISYMAVIIQFCWHFFTIAARVITFALFASVFQLYFGIFIVLHWCIMTFWIVHCETEFCITKWEEIVFDMVVGIIYIFSWFNVKEGRTRCRLFIYYFVILLENTALSALWYLYKAPQIADAFAIPALCVVFSSFLTGVVFMLMYYAFFHPNGPRFGQSPSCACDDPATAFSLPPEVATSTLRSISNNRSVASDRDQKFAERDGCVPVFQVRPTAPPTPSSRPPRIEESVIKIDLFRNRYPAWERHVLDRSLRKAILAFECSPSPPRLQYKDDALIQERLEYETTL

SEQ ID NO: 29 - DNA, 3727 bp, Rattus norvegicus
atgggtagagccccagggccttcgcatttctccaggctggggtttgccagtacagcatccctgaggctgccctctccttatcccgagggcccgccctctgctgccggctttgctttaggtgttccagccctacaggtcctctgccacccaggatctccaaagcatggcacgcccaccaccgctgctagtacagaagcccagcttcctagttgaagcgtgctgttcaccctcgccggcaacacacctagcaccgtaccacacccaaccaggtgcccgaactcccagtacaatacaaagagacctgctcttccccatccctcgccgctgccacgcccgctcgagtccacggccccctgccctcggcggtggcccaacacagagactccaacacgcggcgcgctctgcccaccccatcccccccagcgtcaaggaaatccacccaacgttttccgaaatcccacgagcccgggcctccgactgctgtgctgctgccctcggcgtccagcactggccagcccggcacccccacccgccgctcccctcgatctcgctcgctgtggactactacctgctcggccagcgctggtggtttgggctcaccctgttcttcgtggttctgggctcgctctctgtgcaagtgttcagcttccggtggtttgtgcacgatttcagcaccgaggacagcgccacgaccaccgcctccacctgccagcagcctggagcggattgcaagaccgtggtcagcagtgggtctgcagccggggaaggcgaggctcgtccttccacgccgcagaggcaagcatccaacgccagcaagagcaacatcgccgccaccaacagcggaagcaacagcaacggggccaccaggaccagcggcaaacacaggtctgcgtcctgctccttctgcatctggctcctgcagtcactcatccacatcttgcagctcgggcaagtctggaggtatttgcacacaatatacttaggtatccggagccggcagagcggggagagcagtaggtggcggttttactggaagatggtgtacgagtatgcagatgtgagcatgctgcacctgctggccacctttctggaaagtgcgccacaactggtcctgcagctctgcataattgtacagactcacagcttacaggccctccaaggttttacagcagcagcctcccttgtgtccttggcttgggccctagcctcctaccagaaggctcttcgggactcccgagatgacaaaaagcctatcagctacatggctgtcatcatccagttctgctggcatttcttcaccattgctgccagggtcatcacattcgccctctttgcctcggttttccagctgtattttgggatattcattgtcctccactggtgcatcatgaccttctggattgtccactgtgagacagaattctgtatcaccaaatgggaagagattgtgtttgacatggtggtgggtatcatctacatcttcagttggttcaatgtcaaggaaggcaggacacgctgcaggctgttcatttactattttgtaatccttttggaaaatacagccttgagtgcactctggtacctctacaaagctccccagattgcggatgcatttgccatccctgcattgtgcgtggttttcagcagctttttaacaggtgtcgtttttatgctgatgtactatgccttcttccatcccaatgggcccagatttgggcagtcaccaagttgtgcttgtgacgaccctgccactgccttctctatgcctccagaagtagccacaagcacactacggtccatctctaacaaccgcagtgttgccagtgaccgtgatcagaaatttgcagagcgggatggatgtgtacctgtgtttcaggtgagaccaactgcaccacctactccatcatctcgaccaccgcggattgaagaatcagtcattaaaattgacctgttcaggaatagatatccagcatgggagagacatgtgttggaccgaagcctgagaaaggccattttagcctttgaatgttccccatctcctccaaggctgcagtacaaagacgatgcccttattcaggagaggctggaatatgaaaccactttataaaacacaaagaaccgtaatgtccatataaaggggtaacagcagggctgaggcaataatgacaccttatccaagagtagggcaatgagctatatgttcttagtccaaacattgtcacggtatggtttgatcttccatcagctgactgcctgctgccggtgagcattcaagccagtagtgctgagagtttcttactccgctgaaaggggcgatgtcagcttagtatgactgttgctacaaattcctccagcacaggcttggggcacattcactggaggataacattattgtgaggaaatgttgcctctaatcattagggtattttaatggagtttactaatctttgcataaatatgttcataccaccaccaccaccacccctctatcaaaggagttaaggtggagctggagagatgactcagtagttaagagcactgactgctcttgcaaagaacccaggcttgagccccagcatccatttgatagttcactacaactctaattccagaatatccaatgcccatttttggcctcctcaggcactgcactcacatggtgcacagacatatatgcaggcaaaatacccatacacataaaataaaaataaatctccaaaaacaaaacaaaacaaaaacaaaaaaaagtttaggtgatttccttgatgcaaagctcacaacagactccaagaagaaagcaacatgcttggaatgaccctagaaaccattctctcctttgcaaaccagtagcatcaatgacaaaacctgtgcagtggacagcaagactcaagtgtttacactgatactagcatcgatgggatgattctttttctacgcatttcaggatttgttttttacttttaagttttgcagatgagaacattctttatgacagaaatcctatgcagcacatgtatggcttttgaagagaccaaggagctcaatattcatccgtgatgtaaattaaatgctaatcatgattcagtattcaattgcaaaaataaaatttatatacaaagagccatggcgggagggacagaatgagaatcagattctcaggtgtgtgcatctcctattgaaatacacccacaaagccacggtcgagaaaaagggactgtttccaggtctgtttctaggtgcaggatgagcacgggtcagcaggtgtgattccggaaaccacatgccacacctttctagtgccaaacttcgttcaatcacagaactgatacggtattcccccagactgagaataagtggtgtcccaaaacagacaaggacagaataatcaggttcttggctgtatacagacttaccctcttcccatccaaggtcaaagcgatgtgtctactagagactttgggacaccttttagcaagcgagtgcatacagatgcaatgtgtatgctatcaaaaataaaaactgcctggactgcttgagggcttaccactccatcagctaagatttgtatgtgaatcatctgtaaagttgtgcttttacaagcttctgagttttaaatacctccatacagcaagtaaacattcccgctttctgttcttggtgtcattggtcatggtgctttttgttgcattaaaagtgccggtcaaactttaaaaaaaaaaaaaaaaaa

SEQ ID NO: 30 - protein, 647 aa, Rattus norvegicus
MARPPPLLVQKPSFLVEACCSPSPATHLAPYHTQPGARTPSTIQRDLLFPIPRRCHARSSPRPPALGGGPTQRLQHAARSAHPIPPSVKEIHPTFSEIPRARASDCCAAALGVQHWPARHPHPPLPSISLAVDYYLLGQRWWFGLTLFFVVLGSLSVQVFSFRWFVHDFSTEDSATTTASTCQQPGADCKTVVSSGSAAGEGEARPSTPQRQASNASKSNIAATNSGSNSNGATRTSGKHRSASCSFCIWLLQSLIHILQLGQVWRYLHTIYLGIRSRQSGESSRWRFYWKMVYEYADVSMLHLLATFLESAPQLVLQLCIIVQTHSLQALQGFTAAASLVSLAWALASYQKALRDSRDDKKPISYMAVIIQFCWHFFTIAARVITFALFASVFQLYFGIFIVLHWCIMTFWIVHCETEFCITKWEEIVFDMVVGIIYIFSWFNVKEGRTRCRLFIYYFVILLENTALSALWYLYKAPQIADAFAIPALCVVFSSFLTGVVFMLMYYAFFHPNGPRFGQSPSCACDDPATAFSMPPEVATSTLRSISNNRSVASDRDQKFAERDGCVPVFQVRPTAPPTPSSRPPRIEESVIKIDLFRNRYPAWERHVLDRSLRKAILAFECSPSPPRLQYKDDALIQERLEYETTL

SEQ ID NO: 31 - protein, 4 aa, Artificial Sequence
  Description of Artificial Sequence Synthetic peptide
IGNR

SEQ ID NO: 32 - protein, 4 aa, Artificial Sequence
  Description of Artificial Sequence Synthetic peptide
VANR

SEQ ID NO: 33 - protein, 4 aa, Artificial Sequence
  Description of Artificial Sequence Synthetic peptide
IEPD

SEQ ID NO: 34 - protein, 4 aa, Artificial Sequence
  Description of Artificial Sequence Synthetic peptide
VEPD

SEQ ID NO: 35 - protein, 4 aa, Artificial Sequence
  Description of Artificial Sequence Synthetic peptide
IETD

SEQ ID NO: 36 - protein, 4 aa, Artificial Sequence
  Description of Artificial Sequence Synthetic peptide
IQAD

SEQ ID NO: 37 - protein, 4 aa, Artificial Sequence
  Description of Artificial Sequence Synthetic peptide
PTSY

SEQ ID NO: 38 - protein, 4 aa, Artificial Sequence
  Description of Artificial Sequence Synthetic peptide
YRFK

SEQ ID NO: 39 - protein, 4 aa, Artificial Sequence
  Description of Artificial Sequence Synthetic peptide
KVPL

SEQ ID NO: 40 - protein, 4 aa, Artificial Sequence
  Description of Artificial Sequence Synthetic peptide
WEHD

SEQ ID NO: 41 - protein, 4 aa, Artificial Sequence
  Description of Artificial Sequence Synthetic peptide
FEAD

SEQ ID NO: 42 - protein, 4 aa, Artificial Sequence
  Description of Artificial Sequence Synthetic peptide
YVHD

SEQ ID NO: 43 - protein, 4 aa, Artificial Sequence
  Description of Artificial Sequence Synthetic peptide
LESD

SEQ ID NO: 44 - protein, 4 aa, Artificial Sequence
  Description of Artificial Sequence Synthetic peptide
WEHD

SEQ ID NO: 45 - protein, 4 aa, Artificial Sequence
  Description of Artificial Sequence Synthetic peptide
LEHD

SEQ ID NO: 46 - protein, 4 aa, Artificial Sequence
  Description of Artificial Sequence Synthetic peptide
WEHD

SEQ ID NO: 47 - protein, 4 aa, Artificial Sequence
  Description of Artificial Sequence Synthetic peptide
LEHD

SEQ ID NO: 48 - protein, 4 aa, Artificial Sequence
  Description of Artificial Sequence Synthetic peptide
DEVD

SEQ ID NO: 49 - protein, 4 aa, Artificial Sequence
  Description of Artificial Sequence Synthetic peptide
DGPD

SEQ ID NO: 50 - protein, 4 aa, Artificial Sequence
  Description of Artificial Sequence Synthetic peptide
DEPD

SEQ ID NO: 51 - protein, 4 aa, Artificial Sequence
  Description of Artificial Sequence Synthetic peptide
DELD

SEQ ID NO: 52 - protein, 4 aa, Artificial Sequence
  Description of Artificial Sequence Synthetic peptide
DEED

SEQ ID NO: 53 - protein, 4 aa, Artificial Sequence
  Description of Artificial Sequence Synthetic peptide
DEVD

SEQ ID NO: 54 - protein, 4 aa, Artificial Sequence
  Description of Artificial Sequence Synthetic peptide
DEHD

SEQ ID NO: 55 - protein, 4 aa, Artificial Sequence
  Description of Artificial Sequence Synthetic peptide
VEHD

SEQ ID NO: 56 - protein, 4 aa, Artificial Sequence
  Description of Artificial Sequence Synthetic peptide
VEID

SEQ ID NO: 57 - protein, 4 aa, Artificial Sequence
  Description of Artificial Sequence Synthetic peptide
LETD

SEQ ID NO: 58 - protein, 4 aa, Artificial Sequence
  Description of Artificial Sequence Synthetic peptide
LEHD

SEQ ID NO: 59 - protein, 4 aa, Artificial Sequence
  Description of Artificial Sequence Synthetic peptide
DETD

SEQ ID NO: 60 - protein, 4 aa, Artificial Sequence
  Description of Artificial Sequence Synthetic peptide
VGPD

SEQ ID NO: 61 - protein, 25 aa, Artificial Sequence
  Description of Artificial Sequence Synthetic peptide
  SITE (1)..(25): This sequence may encompass 1-5 “Gly Gly Gly Gly Ser” repeating units
  See specification as filed for detailed description of substitutions and preferred embodiments
GGGGSGGGGSGGGGSGGGGSGGGGS

SEQ ID NO: 62 - DNA, 1708 bp, Homo sapiens
cttttgaaattctaaattctgatgcagaacgtatcagtgaaactccctcccactgtctcttgtattagcatcaaggaagcgagaaaaaataagcagcaccctgagaatggagacagtgtttgaagagatggatgaagaaagcacaggaggagtttcatcttcgaaagaagaaatagtccttggccagagactccatctaagctttccttttagcattatcttctcaactgttctctactgtggtgaggttgcctttggtttatacatgtttgaaatttatcgaaaagctaatgacacattctggatgtcatttaccatcagctttattattgtgggggcaattttggatcaaattatcctgatgtttttcaacaaagacttgaggagaaataaggctgcattacttttttggcacattcttcttttaggacctattgtgaggtgtttgcacaccattagaaattaccacaaatggttgaaaaatcttaaacaggagaaggaagagactcaagttagcatcacaaagagaaacacgatgctggaaagggagattgcattctcaatccgggataatttcatgcagcagaaggctttcaagtacatgtcagtgattcaggcttttctcggttctgttccacaattaattttgcagatgtatatcagtctcactatacgagaatggcctttgaatagagcattgctgatgacattttccctgttatcagttacttatggggccattcgctgcaatatactggccatccagatcagcaatgatgatactaccattaagctaccgccgatagaattcttctgtgtcgtgatgtggcgttttttggaggttatctcacgtgtagtgactctggcatttttcattgcatctctgaaactgaagagcctacccgttttgttaatcatatattttgtatcattgttggcaccgtggctggagttttggaaaagtggagctcatcttcctggcaacaaagaaaataattccaatatggtgggtacagtactgatgcttttcttgatcacactgctatatgctgccatcaacttctcctgctggtcagcagtgaaactgcagttgtcagatgacaaaataattgacgggagacagaggtggggccatagaatcctacactacagctttcagtttttagaaaatgtgataatgatattggtatttaggttctttggagggaaaactttgctgaattgttgtgactcattaattgccgtgcagctcatcataagctacctattggccactggctttatgctcctcttctatcagtatttgtacccatggcagtcaggcaaagtgttgccaggacgtactgaaaatcagccagaagcaccgtactattatgtaaacatcgagaaaactgaaaagaataaaaataagcagctgaggaattactgtcactcctgcaatagggttggatatttttcaatcagaaaaagtatgacatgttcataaaatatacatatatactttcacagaacaatgagtaaagatgctgaatgtgacttgttaagaggctcttaaatttaaaaaatatacacagcaaaatcttggaagtggtttctaataaaattcatttatgttctcctgtgaacgtgccttagtaatttttgttttcttaactataattatacaattcattaaataaaacaaaataaaaaaaaaaaaaaaaaaaaa

SEQ ID NO: 63 - protein, 459 aa, Homo sapiens
METVFEEMDEESTGGVSSSKEEIVLGQRLHLSFPFSIIFSTVLYCGEVAFGLYMFEIYRKANDTFWMSFTISFIIVGAILDQIILMFFNKDLRRNKAALLFWHILLLGPIVRCLHTIRNYHKWLKNLKQEKEETQVSITKRNTMLEREIAFSIRDNFMQQKAFKYMSVIQAFLGSVPQLILQMYISLTIREWPLNRALLMTFSLLSVTYGAIRCNILAIQISNDDTTIKLPPIEFFCVVMWRFLEVISRVVTLAFFIASLKLKSLPVLLIIYFVSLLAPWLEFWKSGAHLPGNKENNSNMVGTVLMLFLITLLYAAINFSCWSAVKLQLSDDKIIDGRQRWGHRILHYSFQFLENVIMILVFRFFGGKTLLNCCDSLIAVQLIISYLLATGFMLLFYQYLYPWQSGKVLPGRTENQPEAPYYYVNIEKTEKNKNKQLRNYCHSCNRVGYFSIRKSMTCS

SEQ ID NO: 64 - protein, 10 aa, Artificial Sequence
  Description of Artificial Sequence Synthetic peptide
NLVPMVATVQ

SEQ ID NO: 65 - DNA, 97 bp, Artificial Sequence
  Description of Artificial Sequence Synthetic oligonucleotide
  CDS (1)..(96)
tattatcattggctgcacccgagttgctgctggaagcctgacccggaccaggttgatggagcgcggtccttgctctccccagaaggctaccagttgc

SEQ ID NO: 66 - protein, 32 aa, Artificial Sequence
  Description of Artificial Sequence Synthetic polypeptide
YYHWLHPSCCWKPDPDQVDGARSLLSPEGYQL

SEQ ID NO: 67 - DNA, 108 bp, Artificial Sequence
  Description of Artificial Sequence Synthetic polynucleotide
  CDS (1)..(108)
tattatcattggctgcacccgagttgctgctggaagcctgacccggtgggacctgattttggtagagaattcgcgcggtccttgctctccccagaaggctaccagttg

SEQ ID NO: 68 - protein, 36 aa, Artificial Sequence
  Description of Artificial Sequence Synthetic polypeptide
YYHWLHPSCCWKPDPVGPDFGREFARSLLSPEGYQL

SEQ ID NO: 69 - DNA, 120 bp, Artificial Sequence
  Description of Artificial Sequence Synthetic polynucleotide
  CDS (1)..(120)
tattatcattggctgcacccgagttgctgctggaagcctgacccgggatcggtgggacctgattttggtagagaattcggcagtgcgcggtccttgctctccccagaaggctaccagttg

SEQ ID NO: 70 - protein, 40 aa, Artificial Sequence
  Description of Artificial Sequence Synthetic polypeptide
YYHWLHPSCCWKPDPGSVGPDFGREFGSARSLLSPEGYQL

(19) United States
(12) Patent Application Publication
(10) Pub. No.: US 2023/0287390 A1
(43) Pub. Date: Sep. 14, 2023

(54) COMPOSITIONS AND METHODS FOR IDENTIFYING EPITOPES

(71) Applicant: TScan Therapeutics, Inc., Waltham, MA (US)

(72) Inventors: Yifan WANG, Arlington, MA (US); Andrew P. Ferretti, Waltham, MA (US); Nancy Nabilsi, Lynnfield, MA (US); Gavin MacBeath, Wakefield, MA (US); Tomasz Kula, Brookline, MA (US)

(21) Appl. No.: 18/011,577

(22) PCT Filed: Jul. 20, 2021
(86) PCT No.: PCT/US2021/042311

§ 371 Date: Dec. 20, 2022

Related U.S. Application Data
(60) Provisional application No. 63/055,766, filed on Jul. 23, 2020.

(51) Int. Cl. C12N 15/10; C12N 9/00

(52) U.S. Cl. CPC C12N 15/1037; C12N 9/00

(57) ABSTRACT

Provided herein are methods and compositions for identifying epitopes by using reporters of phospholipid scramblase.

### CROSS-REFERENCE TO RELATED APPLICATIONS

This application claims the benefit of priority to U.S. Provisional Application Ser. No. 63/055,766, filed on 23 Jul. 2020; the entire contents of said application are incorporated herein in their entirety by this reference.

### BACKGROUND OF THE INVENTION

Phosphatidylserine (PS) is a well-established marker for cells undergoing apoptosis, and commercial reagents are available that use PS for the detection, enrichment, and/or removal of dying cells. PS is normally restricted to the inner leaflet of cell membrane lipid bi-layers and healthy cells are PS negative according to Annexin V staining. However, during apoptosis, apoptosis-mediated scramblases like XKR8 promote the translocation of PS to the outer leaflet of cell membrane lipid bi-layers, such as the cell surface membrane lipid bi-layer that becomes positive for PS according to Annexin V staining. Such scramblases maintain an inactive state in living cells and transition to a catalytically active state via caspase-mediated cleavage during cell apoptosis.

Cytotoxic lymphocytes like cytotoxic T cells use receptors like T cell receptors (TCRs) to recognize cognate antigens presented by target cells on MHC molecules. Cytotoxic lymphocyte activation results in the delivery of granules and agents contained therein, such as perforin and serine proteases like granzymes, to the target cells, which eventually leads to the killing of target cells via activation of APC-derived caspases. Granzyme B is one such cytotoxic protein, which exhibits protease activity and degrades various target cell proteins that contain the granzyme B cleavage motif. This feature of granzyme B has led to the development of cytoplasmic fluorescent granzyme reporters that allow for the identification of target cells recognized by T cells through cell sorting for a generated fluorescent signal. However, the use of such reporters in large-scale screens is limited by the processing speed and scale of cell sorting instruments.

Accordingly, there is a need for additional reporters that are capable of increasing the efficiency and sensitivity of target cell identification and enabling more effective T cell antigen discovery.

### SUMMARY OF THE INVENTION

The present invention is based, at least in part, on the provision of reporters of phospholipid scrambling comprising a scramblase comprising a serine protease cleavage site and/or a caspase cleavage site that activates the scramblase upon cleavage by the serine protease and/or the caspase. Such reporters are useful for enhancing the presentation of phosphatidylserine (PS) on target cells upon recognition by cytotoxic T cells and/or natural killer (NK) cells. This may occur when cytotoxic T cells and/or NK cells recognize antigen-presenting cells (APCs) expressing a peptide antigen-major histocompatibility complex (pMHC) complex via cell surface receptors and transfer serine proteases like granzymes into the APCs. Such APCs comprising the reporters of phospholipid scrambling express activated scramblase when cleaved by the serine proteases and/or downstream caspases at serine protease cleavage sites and/or caspase cleavage sites, respectively, present in the scramblase and maintaining the cleavable portion of the scramblase conferring inhibition of scramblase activity until cleaved. The activated scramblase is capable of promoting the translocation of phosphatidylserine (PS) to the outer leaflet of a cell membrane lipid bi-layer, such as the cell surface membrane bi-layer. Since PS is normally restricted to the inner leaflet of the membrane bi-layer, cells presenting PS on the outer leaflet of the membrane bi-layer like the cell surface indicates activation of the reporter and corresponding recognition of the expressed pMHC complex by a cytotoxic T cell and/or NK cell. This system allows for large-scale, rapid detection of APCs engaged by cytotoxic T cells and/or NK cells from among 1) a large population of APCs collectively expressing a large diversity of different peptide antigens and MHC complexes and 2) a large population of cytotoxic T cells and/or NK cells having affinity for a large diversity of different peptide antigens and MHC complexes. In addition, the antigens of the recognized pMHC complexes may be determined, such as by isolating APCs having reporter signal away from other APCs and identifying the antigens expressed therein (e.g., extracting antigen-encoding nucleic acids, optionally amplifying such nucleic acids, and sequencing such nucleic acids). Reporter compositions, as well as systems comprising such reporter compositions and methods using such reporter compositions, are provided herein.

In one aspect, a cell comprising a reporter of phospholipid scrambling, wherein the reporter of phospholipid scrambling comprises a scramblase comprising a serine protease cleavage site and/or a caspase cleavage site that activates the scramblase upon cleavage by the serine protease and/or the caspase, is provided.

In another aspect, a library of cells described herein, wherein the cells comprise different exogenous nucleic acids encoding one or more candidate antigens to thereby represent a library of candidate antigens expressed and presented with MHC class I and/or MHC class II molecules, is provided.

In still another aspect, a reporter of phospholipid scrambling comprising a scramblase comprising a serine protease cleavage site and/or a caspase cleavage site that activates the scramblase upon cleavage by the serine protease and/or the caspase, is provided.

In yet another aspect, a nucleic acid that encodes a reporter described herein, optionally wherein the nucleic acid comprises a nucleotide sequence having at least 80% identity with a nucleic acid sequence described herein, is provided.

In another aspect, a vector that comprises a nucleic acid that encodes a reporter described herein, is provided.

In still another aspect, a cell that comprises a nucleic acid or vector described herein, is provided.

In yet another aspect, a method of making a recombinant cell comprising (i) introducing in vitro or ex vivo a recombinant nucleic acid or a vector described herein into a host cell, (ii) culturing in vitro or ex vivo the recombinant host cell obtained, and (iii), optionally, selecting the cells which express said recombinant nucleic acid or vector, is provided.

In another aspect, a system for detection of an antigen presented by an antigen presenting cell (APC) that is recognized by a cyotoxic lymphocyte, optionally wherein the cytotoxic lymphocyte is a cytotoxic T cell and/or natural killer (NK) cell, comprising: a) an APC comprising a cell described herein and b) a cytotoxic lymphocyte, is provided.

In still another aspect, a method for identifying an antigen that is recognized by a cytotoxic T cell and/or NK cell, comprising a) contacting an APC or a library of APCs described herein with one or more cytotoxic lymphocytes, optionally wherein the cytotoxic lymphocytes are cytotoxic T cells and/or NK cells, under conditions appropriate for recognition by the cytotoxic lymphocytes of antigen presented by the APC or the library of APCs; b) identifying APC(s) having an activated scramblase upon cleavage by the serine protease originating from a cytotoxic lymphocyte, and/or the caspase, in response to recognition by the cytotoxic lymphocyte of antigen presented by the cell or the library of cells; and c) determining the nucleic acid sequence encoding the antigen from the cell identified in step b), thereby identifying the antigen that is recognized by the cytotoxic lymphocyte, is provided.

As described further herein, numerous embodiments are provided that can be applied to any aspect of the presevnt invention and/or combined with any other embodiment described herein.

### BRIEF DESCRIPTION OF THE DRAWINGS

FIG. 1 shows a schematic diagram of a granzyme-activated infrared fluorescent protein (IFP) reporter and a granzyme-activated scramblase reporter.

FIG. 2 shows engineered granzyme B cleavage sites in the scramblase reporter constructs.

FIG. 3A shows that scramblase enhances IFP⁺ Annexin V⁺ enrichment after 1 hour.

FIG. 3B shows that scramblase enhances IFP⁺ Annexin V⁺ enrichment after 4 hours.

FIG. 4 shows the Annexin V column-based enrichment of YW3 granzyme scramblase/IFP-GzB double reporter cells in the context of a large-scale screen.

### DETAILED DESCRIPTION OF THE INVENTION

The present invention is based, at least in part, on the generation of reporters of phospholipid scrambling comprising a scramblase comprising a serine protease cleavage site and/or a caspase cleavage site that activates the scramblase upon cleavage by the serine protease and/or the caspase. In representative examples, it was determined that such reporters enhance the presentation of phosphatidylserine (PS) on target cells upon T cell recognition, and enable efficient Annexin V-based enrichment of the target cells. This enables antigen discovery at a higher scale and efficiency.

Accordingly, the present invention relates, in part, to the reporters of phospholipid scrambling, as well as nucleic acids, vectors, cells, libraries, systems, and other compositions described herein, as well as methods of using such compositions described herein.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “administering” means providing a pharmaceutical agent or composition to a subject, and includes, but is not limited to, administering by a medical professional and self-administering.

The term “antigen” refers to a molecule capable of inducing an immune response in a host organism, and is specifically recognized by T cells. In some embodiments, an antigen is a peptide. As used herein, the term “candidate antigen” refers to a peptide encoded by an exogenous nucleic acid introduced into the target cells intended for use in the screening methods described herein. Libraries, as described herein, comprise target cells which include introduced candidate antigens.

The term “antigen-presenting cells” or “APC” relates to cells that display peptide antigen in complex with the major histocompatibility complex (MHC) on its surface. APC are also referred to herein as APC targets, target cells, or target APC. Any cell is suitable as an antigen-presenting cell in accordance with the present invention, as long as it expresses an MHC and presents an antigen (e.g., any cell that can present antigen via MHC class I and/or MHC class II to an immune cell (e.g., a cytotoxic immune cell)). Cells that have in vivo the potential to act as antigen presenting cells include, for example, professional antigen presenting cells like monocytes, dendritic cells, Langerhans cells, macrophages, B cells, as well as other antigen presenting cells (activated epithelial cells, keratinocytes, endothelial cells, astrocytes, fibroblasts, oligodendrocytes, glial cells, pancreatic beta cells, and the like). Such cells may be employed in accordance with the present invention after transfection or transformation with a library encoding candidate antigens as described herein (e.g., modified to present a candidate antigen via expression of an exogenous nucleic acid stably inserted into the genome of the APC). Also, cells not endogenously expressing MHC may be employed, in which case suitable MHC are to be transformed or transfected into said cells. Cells may be primary cells or cells of a cellin line. Representative, non-limiting examples of cells suitable for use as APCs include HEK293, HEK293T, U20S, K562, MelJuso, MDA-MB231, MCF7, NTERA2a, LN229, dendritic, primary T cells, and primary B cells).

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “caspase” refers to a family of protease enzymes playing essential roles in programmed cell death. Caspases are endoproteases that hydrolyze peptide bonds in a reaction that depends on catalytic cysteine residues in the caspase active site and occurs only after certain aspartic acid residues in the substrate. Although caspase-mediated processing can result in substrate inactivation, it may also generate active signaling molecules that participate in ordered processes such as apoptosis and inflammation. Accordingly, caspases have been broadly classified by their known roles in apoptosis (caspase-3, -6, -7, -8, and -9 in mammals), and in inflammation (caspase-1, -4, -5, -12 in humans and caspase-1, -11, and -12 in mice). The functions of caspase-2, -10, and -14 are less easily categorized. Caspases involved in apoptosis have been subclassified by their mechanism of action and are either initiator caspases (caspase-8 and -9) or executioner caspases (caspase-3, -6, and -7). Caspases are initially produced as inactive monomeric procaspases that require dimerization and often cleavage for activation. Assembly into dimers is facilitated by various adapter proteins that bind to specific regions in the prodomain of the procaspase. The exact mechanism of assembly depends on the specific adapter involved. Different caspases have different protein-protein interaction domains in their prodomains, allowing them to complex with different adapters. For example, caspase-1, -2, -4, -5, and -9 contain a caspase recruitment domain (CARD), whereas caspase-8 and -10 have a death effector domain (DED).

The caspase-3 subfamily includes caspase-3, -6, -7, -8, and -10. Among this family, caspase-3 shares highest homology with caspase-7 and both have short prodomains; whereas caspase-6, -8, and -10 have long prodomains. Caspase-3 has been shown to be a major execution caspase that acts downstream in the apoptosis pathway and is involved in cleaving important substrates such as ICAD (inhibitor of caspase activated DNase), which activates the apoptotic DNA ladder-forming activity of CAD (caspase activated DNase). The major route of activating short prodomain caspases is through direct proteolytic processing. Two known pathways that can activate procaspase-3 are through proteolytic cleavage by caspase-8 and -9. Thus, caspase-8 and -9 have been known as the two major upstream activators of caspase-3. Structure-function relationships describing caspase structure/sequence and activity are well-known in the art (see, e.g., Li et al. (2008) Oncogene 27:6194-6206 and Mcllwain et al. (2013) Cold Spring Haab. Perspect Biol. 2013; 5:a008656).

The term “caspase-activated deoxyribonuclease (CAD)” or “DNA fragmentation factor subunit beta (DFFB)” refers to a nuclease that induces DNA fragmentation and chromatin condensation during apoptosis. It is encoded by the DFFB gene in humans. It is usually an inactive monomer inhibited by inhibitor of caspase-acivated deoxyribonuclease (ICAD), and cleaved before dimerization. The apoptotic process is accompanied by shrinkage and fragmentation of the cells and nuclei and degradation of the chromosomal DNA into nucleosomal units. DNA fragmentation factor (DFF) is a heterodimeric protein of 40-kD (DFF40, DFFB, or CAD) and 45-kD (DFF45, DFFA, or ICAD) subunits. DFFA is the substrate for caspase-3 and triggers DNA fragmentation during apoptosis. DFF becomes activated when DFFA is cleaved by caspase-3. The cleaved fragments of DFFA dissociate from DFFB, the active component of DFF. DFFB has been found to trigger both DNA fragmentation and chromatin condensation during apoptosis.

The term “caspase-activated deoxyribonuclease (CAD)-mediated DNA degradation” refers to internucleosomal degradation of genomic DNA by the caspase-activated deoxyribonuclease (CAD).

The term “cleavage site,” in some embodiments, refers to a stretch of amino acid sequence that recognized and cleaved by a protease, such as a “serine protease cleavage site” (e.g., members of the granzyme family) or that of a caspase. For example, amino acid recognition motifs of members of the granzyme family are known in the art (see, e.g., Mahrus et al. (2005) Chem. Biol. 12:567-577, the MEROPS database described in Rawlings et al. (2010) Nucl. Acids Res. 38:D227-D233, and Bao et al. (2019) Briefings Bioinformatics 20:1669-1684). Exemplary, non-limiting cleavage sites for serine proteases (e.g., members of the granzyme family) are shown in Table 1A below.

TABLE 1A

| Serine Protease Name | Cleavage Site Sequence | Sequence ID No. |
|---|---|---|
| Granzyme A | IGNR | 31 |
| Granzyme A | VANR | 32 |
| Granzyme B | IEPD | 33 |
| Granzyme B | VEPD | 34 |
| Granzyme B | VGPDFGREF or VGPD | 4 |
| Granzyme B | IETD | 35 |
| Granzyme B | IQAD | 36 |
| Granzyme H | PTSY | 37 |
| Granzyme K | YRFK | 38 |
| Granzyme M | KVPL | 39 |

Similarly, the term “caspase cleavage site” refers to a stretch of sequence that recognized and cleaved by caspase (e.g., caspase 3, 7, 8 or 9). The amino acid recognition motifs of members of the caspase family are well-known in the art (see, e.g., Li and Yuan (2008) Oncogene 27:6194-6206). For example, representative, exemplary tetrapeptide substrate sequences for caspase-1- to -11 have been determined and are well-known in the art (see, e.g., Thornberry et al. (1997) J. Biol. Chem. 272: 17907-17911 and Kang et al. (2000) J Cell Biol 149: 613-622). To date, almost 400 substrates for mammalian caspases have been reported in the literature, which are compiled into an online database ‘CASBAH’ (available on the World Wide Web at casbah.ie) (Luthi and Martin (2007) Cell Death Differ. 14:641-650). Exemplary, non-limiting cleavage sites for caspases are shown in Table 1B below.

TABLE 1B

| Caspase Name | Cleavage Site Sequence | Sequence ID No. |
|---|---|---|
| Caspase 1 | WEHD | 40 |
| Caspase 1 | FEAD | 41 |
| Caspase 1 | YVHD | 42 |
| Caspase 1 | LESD | 43 |
| Caspase 4 | WEHD | 44 |
| Caspase 4 | LEHD | 45 |
| Caspase 5 | WEHD | 46 |
| Caspase 5 | LEHD | 47 |
| Caspase 3 | DEVD | 48 |
| Caspase 3 | DGPD | 49 |
| Caspase 3 | DEPD | 50 |
| Caspase 3 | DELD | 51 |
| Caspase 3 | DEED | 52 |
| Caspase 7 | DEVD | 53 |
| Caspase 2 | DEHD | 54 |
| Caspase 6 | VEHD | 55 |
| Caspase 6 | VEID | 56 |
| Caspase 8 | LETD | 57 |
| Caspase 9 | LEHD | 58 |
| C. elegans CED-3 | DETD | 59 |

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “control” refers to a control reaction which is treated otherwise identically to an experimental reaction, with the exception of one or more critical factors. A control may be a cell which is identical, but is not exposed to an activating molecule (e.g., an activating cytotoxic lymphocyte, such as a cytotoxic T cell and/or an NK cell). Alternatively, a control may be a cell which is exposed to an activating molecule but which lacks a reporter molecule (and may be otherwise identical to experimental cells). An appropriate control is determined by the skilled practitioner.

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in some embodiments, at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of a condition in the subject) for a subject that is started, modified and/or ended based or essentially based or at least partially based on the results of the analysis according to the present invention. The determination may, in addition to the results of analyses consistent with methods encompassed by the present invention, be based on personal characteristics of the subject to be treated. In most cases, the actual determination of the suitable treatment regimen for the subject will be performed by the attending physician or doctor.

The term “exogenous” refers to material originating external to or extrinsic to a cell (e.g., nucleic acid from outside a cell inserted into the cellular genome is considered exogenous nucleic acid).

The term “granzymes” refers to a family of serine proteases expressed by cytotoxic lymphocytes, suc as cytotoxic T lymphocytes and natural killer (NK) cells, that protect higher organisms against viral infection and cellular transformation. For example, following receptor-mediated conjugate formation between a granzyme-containing cell and an infected or transformed target cell, granzymes enter the target cell via endocytosis and induce apoptosis. Five different granzymes have been described in humans: granzymes A, B, H, K and M. In mice, clear orthologues of four of these granzymes (A, B, K and M) can be found, and granzyme C seems is believed to be the murine orthologue of granzyme H. The murine genome encodes several additional granzymes (D, E, F, G, L and N), of which D, E, F and G are expressed by cytotoxic lymphocytes. In some embodiments, granzyme L is encoded by a pseudogene and granzyme N is expressed in the testis.

Granzyme B is the most powerful pro-apoptotic member of the granzyme family. It is responsible for the rapid induction of caspase-dependent apoptosis. Human granzyme-B-mediated apoptosis is in part mediated by mitochondria. To induce mitochondrial changes, granzyme B cleaves the BH3-only pro-apoptotic protein Bid. Upon cleavage, truncated BID translocates to the mitochondria and together with Bax and/or Bak results in release of pro-apoptotic proteins and mitochondrial outer membrane permeabilization. Cytochrome c release is crucial in apoptosome formation and subsequent caspase-9 activation, which in turn cleaves downstream effector caspases. In addition to Bid, granzyme B can induce cytochrome c release by cleavage and inactivation of the anti-apoptotic Bcl-2 family member Mcl-1.

Besides its Bcl-2-family-directed actions, granzyme B can process several caspases, including the effector caspase 3 and initiator caspase 8. Granzyme B has also been reported to process several known caspase substrates directly, such as poly (ADP-ribose) polymerase (PARP), DNA-dependent protein kinase (DNA-PK), ICAD, the nuclear mitotic apparatus protein (NuMa) and lamin B. Although most research has focused on the caspase-related pathways, granzyme B also induces caspase-independent events. Major hallmarks of granzyme B-induced cellular damage are oligonucleosomal DNA fragmentation and mitochondrial damage.

An important pathway to granzyme A-induced damage involves cleavage and inactivation of SET (also known as PHAPII, TAF-Iβ, I2^{PP2A}), which functions as an inhibitor of the DNase activity of the tumor metastasis suppressor NM23-H1. The resulting hallmark of granzyme A-induced damage is single-stranded DNA nicks mediated by NM23-H1. Structure-function relationships describing caspase structure/sequence and activity are well-known in the art (see, e.g., Trapani (2001) Genome Biol. 2:3014.1-3014.7 and Bots and (2006) J. Cell Sci. 119:5011-5014).

The term “GS linker” refers to a linker having a sequence of glycine and serine, such as sequences consisting primarily of stretches of Gly and Ser residues. In some embodiments, the linker has the sequence of (Gly-Ser)ₙ. In some embodiments, the linker has the sequence of Gly-Ser. In some embodiments, the linker as the sequence of (Gly-Gly-Gly-Gly-Ser)ₙ. N is a natural number, such as 1, 2, 3, 4, 5, and the like.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “isolated” refers to a composition that is substantially free of other undesired materials (e.g., nucleic acids, cells, proteins, organelle, cellular material, separation medium, culture medium, etc. as the case may be). In some embodiments, compositions may be separated from cells or other materials present. Such undesired materials may be present in a number of environments, such as in a state where the component naturally occurs (e.g., chromosomal and extra-chromosomal DNA and RNA, cellular components, and the like), during production by recombinant DNA techniques, or chemical precursors or other chemicals when chemically synthesized. In some embodiments, the composition that is isolated may be determined to be substantially free of other undesired materials on a measured basis (e.g., clones, sequence, activity, weight, volume, and the like) such as having less than about 50%, 45%, 40%, 35%, 30%, 25%, 20%, 15%, 10%, 9%, 8%, 7%, 6%, 5%, 4%, 3%, 2%, 1%, or even less, or any range in between, inclusive, such as less than about 5-15%, undesired material. Another way to express substantial freedom of other undesired materials is to determine the composition of interest on a measured basis (e.g., clones, sequence, activity, weight, volume, and the like) such as having greater than about 50%, 55%, 60%, 65%, 70%, 75%, 80%, 85%, 90%, 91%, 92%, 93%, 94%, 95%, 96%, 97%, 98%, 99%, or greater, or any range in between, inclusive, such as greater than about 95-99%, desired composition relative to undesired materials.

The term “K_{D}” is intended to refer to the dissociation equilibrium constant of a particular interaction between associating compositions. For example, the binding affinity between a TCR and a peptide antigen-major histocompatibility complex (pMHC) complex may be measured or determined by standard assays, for example, biophysical assays, competitive binding assays, saturation assays, or standard immunoassays, such as ELISA or RIA.

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

The term “natural killer cell” or “NK cell” refers to a type of cytotoxic lymphocyte derived from a common progenitor as T and B cells. As cells of the innate immune system, NK cells are classified as group I innate lymphocytes (ILCs) and respond quickly to a wide variety of pathological challenges. NK cells are best known for killing virally infected cells, and detecting and controlling early signs of cancer. As well as protecting against disease, specialized NK cells are also found in the placenta and may play an important role in pregnancy. In some embodiments, NK cells use NK cell receptors (NKRs) to recognize peptide antigen-major histocompatibility complex (pMHC) complexes as part of an adaptive immune response (see, for example, Cooper (2018) Proc. Natl. Acad. Sci. 115:11357-11359).

The term “percent identity” between amino acid or nucleic acid sequences is synonymous with “percent homology,” which may be determined using the algorithm of Karlin and Altschul (1990) Proc. Natl. Acad. Sci. U.S.A. 87:2264-2268, modified by Karlin and Altschul (1993) Proc. Natl. Acad. Sci. U.S.A. 90:5873-5877. The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nucleic Acids Res. 25:3389-3402). When utilizing BLAST and Gapped BLAST programs, the default parameters of the respective programs (e.g., XBLAST and NBLAST) are used.

“Homologous,” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, at least about 75%, at least about 90%, or at least about 95% of the nucleotide residue positions of each of the portions are occupied by the same nucleotide residue. In some embodiments, all nucleotide residue positions of each of the portions are occupied by the same nucleotide residue.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “phospholipid” refers to a class of lipids that are a major component of cell membranes. They can form lipid bilayers because of their amphiphilic characteristic. The structure of the phospholipid molecule generally consists of two hydrophobic fatty acid “tails” and a hydrophilic “head” consisting of a phosphate group. The two components are usually joined together by a glycerol molecule. The phosphate groups can be modified with simple organic molecules, such as choline, ethanolamine, or serine. In some embodiments, the phospholipid is phosphatidylserine (PS).

The term “phosphatidylserine” or “PS” refers to a glycerophospholipid which consists of two fatty acids attached in ester linkage to the first and second carbon of glycerol and serine attached through a phosphodiester linkage to the third carbon of the glycerol. PS is a component of the cell membrane, and plays a key role in cell cycle signaling, specifically in relation to apoptosis. PS exposure on the external leaflet of the cell surface membrane is a classic feature of apoptotic cells and acts as an “eat me” signal allowing phagocytosis of post-apoptotic bodies. PS can be detected in a variety of well-known ways, including, but not limited to, biochemical fractionation followed by mass spectrometric identification, and/or use of PS-binding probes (e.g., 2,4,6-trinitrobenzenesulfonate (TNBS)), anti-PS antibodies, Annexin V, fluorescently-labelled PS analogues (e.g., 7-nitro-2-1,3-benzoxadiazol-4-yl (NBD)), peptide-based PS indicator PSP1, and/or discoidin-C2 (GFP-LactC2) (see, for example, Kay and Grinstein (2011) Sensors 11:1744-1755).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

The term “sample” includes samples from biological sources, such as whole blood, plasma, serum, brain tissue, cerebrospinal fluid, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In some embodiments, biological samples comprise cells, such as immune cells and/or antigen-presenting cells. In some embodiments, methods encompassed by the present invention further comprise obtaining a sample, such as from a biological source of interest.

The term “scramblase” refers to a protein responsible for the translocation of phospholipids between the two monolayers of a lipid bilayer of a cell membrane. In some embodiments, the scramblase is a member of the phospholipid scramblase family. Phospholipid scramblases are membrane proteins that mediate calcium-dependent, non-specific movement of plasma membrane phospholipids and phosphatidylserine exposure. The encoded protein contains a low affinity calcium-binding motif and may play a role in blood coagulation and apoptosis. In humans, phospholipid scramblases (PLSCRs) constitute a family of five homologous proteins that are named as hPLSCR1-hPLSCR5. Although PLSCR1 (phospholipid scramblase 1) was once reported to be a scramblase, its molecular properties and the phenotypes of PLSCR-deficient mice and Drosophila ruled PLSCR1 out as a phospholipid scramblase.

In some embodiments, the scramblase is an apoptosis-mediated scramblase rather than a calcium-mediated scramblase. In some embodiments, the scramblase is a member of the Xkr family, such as Xkr8, Xkr4, Xkr9, or Xkr3. In some embodiments, the scramblase is a human scramblase. Xkr8, a membrane protein carrying 10 putative transmembrane segments, was originally identified as a scramblase that is activated by caspase-mediated cleavage during apoptosis. Xkr8 promotes phosphatidylserine exposure on apoptotic cell surface, possibly by mediating phospholipid scrambling Phosphatidylserine is a specific marker only present at the surface of apoptotic cells and acts as a specific signal for engulfment. Xkr8 has no effect on calcium-induced exposure of PS. Xkr8 is activated upon caspase cleavage, suggesting that it does not act prior the onset of apoptosis. Xkr8 belongs to the Xkr family, which has nine and eight members in humans and mice, respectively. Xkr8 carries a well-conserved caspase 3 recognition site in its C-terminal tail region, and its cleavage by caspases 3/7 during apoptosis induces its dimerization to an active scramblase form. It has been shown that not only Xkr8, but also Xkr4, Xkr9, and other scramblases support apoptotic PS exposure when activated via cleavage (Suzuki et al. (2014) J. Biol. Chem. 289:30257-30267; Williamson (2015) Lipid Insights 8:41-44; Ploier et al. (2016) J. Vis. Exp. 115:54635; Suzuki et al. (2016) Proc. Natl. Acad. Sci. U.S.A. 113:9509-9514; Pomorski et al. (2016) Prog. Lipid Res. 64:69-84; Nagata et al. (2016) Cell Death Differ. 23:952-961; Sakuragi et al. (2019) Proc. Natl. Acad. Sci. U.S.A. 116:2907-2912). Like Xkr8, Xkr4 and Xkr9 carry a caspase-recognition site in their C-terminal region, and this site is cleaved during apoptosis to activate the scramblase and expose PS. Xkr8 is ubiquitously expressed in various tissues, and is expressed strongly in the testes. Xkr4 is ubiquitously expressed at low levels, but is strongly expressed in the brain and eyes. Xkr9 is strongly expressed in the intestines. Flies and nematodes carry an Xkr8 ortholog (CG32579 in D. melanogaster, and CED8 in C. elegans). CED8 has a caspase (CED3)-recognition site in its N terminus and is needed for CED3-dependent PS exposure.

Structure-function relationships between apoptosis-mediated scramblase activation and cleavage sites are well-known in the art (see, for example, Suzuki et al. (2014) J. Biol. Chem. 289:30257-30267; Williamson (2015) Lipid Insights 8:41-44; Ploier et al. (2016) J. Vis. Exp. 115:54635; Suzuki et al. (2016) Proc. Natl. Acad. Sci. U.S.A. 113:9509-9514; Pomorski et al. (2016) Prog. Lipid Res. 64:69-84; Nagata et al. (2016) Cell Death Differ. 23:952-961; Sakuragi et al. (2019) Proc. Natl. Acad. Sci. U.S.A. 116:2907-2912). For example, point mutations that prevent PS scramblase activity in apoptosis-mediated scramblases are well-known, such as A46E, S64L, G94R, E141R, L150E, S184V, and D295K mutations in Xkr8. Similarly, mutation of residues Val-35, Glu-141, Gln-163, Ser-184, Ile-216, Val-305, and Thr-309 (such as V35A, Q163T, I216T, V3055, and T309F) (numbering is based on Xkr8), which are conserved among Xkr8, Xkr9, Xkr4, and CED-8, do not prevent PS scramblase activity in apoptosis-mediated scramblases. However, mutation of residues Glu-141 and Ser-184 (such as E141R and S184V) (numbering is based on Xkr8), which are present in Xkr8, Xkr9, Xkr4, and CED-8, do prevent PS scramblase activity in apoptosis-mediated scramblases. Similarly, the structure of cleaved apoptosis-mediated scramblase forms and activation of scramblase activity are well-known. For example, cleavage of apoptosis-mediated scramblases at their endogenous (native) caspase cleavage position, whether with the native caspase cleavage sequence or cleavage sequence of another protease like a serine protease or another caspase, activates scramblase activity. Cleavage C-terminal to such endogenous caspase cleavage positions (e.g., downstream of residues 352-356 of SEQ ID NO: 10) also activates scramblase activity.

The term “Xkr8” is intended to include fragments, variants (e.g., allelic variants), and derivatives thereof. Representative human Xkr8 cDNA and human Xkr8 protein sequences are well-known in the art and are publicly available from the National Center for Biotechnology Information (NCBI). For example, human Xkr8 (NP_060523.2) is encodable by the transcript (NM_018053.4). Nucleic acid and polypeptide sequences of Xkr8 orthologs in organisms other than humans are well-known and include, for example, chimpanzee Xkr8 (NM_001033037.1 and NP_001028209.1), Rhesus monkey Xkr8 (XM_015151522.1 and XP_015007008.1), dog Xkr8 (XM_003638918.4 and XP 003638966.1), cattle Xkr8 (XM 002685687.5 and XP 002685733.1), mouse Xkr8 (NM201368.1 and NP_958756.1), rat Xkr8 (NM_001012099.1 and NP_001012099.1), chicken Xkr8 (NM_001044693.1 and NP_001038158.1), tropical clawed frog Xkr8 (NM_001033944.1 and NP_001029116.1), and zebrafish Xkr8 (NM_001006014.2 and NP 001006014.2). Representative sequences of Xkr8 orthologs are presented below in Table 2A.

Reagents useful for detecting Xkr8 and cleaved forms thereof are known in the art. For example, Xkr8 can be detected using antibodies LS-B12131 (LSBio), DPABH-14044 (Creative Diagnostics), TA330830 and TA330831 (Origene), NBP2-81866 and NBP2-14699 (Novus Biologicals), etc. Some of these Xkr8 antibodies bind to a C-terminal portion of Xkr8, such as Cat. No. ABIN2568972 and Cat. No. ABIN6752928 (antibodies-online.com). Some of these Xkr8 antibodies bind to an N-terminal portion of Xkr8, such as orb45542 (Biorbyt).

The term “Xkr9” is intended to include fragments, variants (e.g., allelic variants), and derivatives thereof. Representative human Xkr9 cDNA and human Xkr9 protein sequences are well-known in the art and are publicly available from the National Center for Biotechnology Information (NCBI). For example, human Xkr9 isoform 1 (NP_001274187.1) is encodable by the transcript variant 2 (NM_001287258.2); human Xkr9 isoform 2 (NP_001011720.1; NP_001274188.1; and NP_001274189.1) is encodable by the transcript variant 1 (NM_001011720.2), transcript variant 3 (NM_001287259.2), and transcript variant 4 (NM_001287260.2). Nucleic acid and polypeptide sequences of Xkr9 orthologs in organisms other than humans are well-known and include, for example, chimpanzee Xkr9 (NM_001033038.1 and NP_001028210.1), Rhesus monkey Xkr9 (XM_028852736.1 and XP_028708569.1), dog Xkr9 (XM_022412238.1 and XP_022267946.1; XM 022412240.1 and XP_022267948.1; XM 022412239.1 and XP_022267947.1; XM 014109283.2 and XP_013964758.1; XM 014109286.2 and XP_013964761.1; XM 022412241.1 and XP_022267949.1; XM 022412244.1 and XP_022267952.1; XM 022412243.1 and XP_022267951.1; XM 022412245.1 and XP_022267953.1; XM_014109287.2 and XP_013964762.1), cattle Xkr9 (XM_002692698.5 and XP_002692744.1), mouse Xkr9 (NM_001011873.2 and NP_001011873.1), rat Xkr9 (NM_001012229.1 and NP_001012229.1), chicken Xkr9 (NM_001034824.1 and NP_001029996.1), tropical clawed frog Xkr9 (NM_001033945.1 and NP_001029117.1), and zebrafish Xkr9 (NM_001012259.1 and NP_001012259.1). Representative sequences of Xkr9 orthologs are presented below in Table 2A.

Reagents useful for detecting Xkr9 and cleaved forms thereof are known in the art. For example, Xkr9 can be detected using antibodies CABT-BL3813 (Creative Diagnostics), NBP1-94164 (Novus Biologicals), Cat #PA5-60711 (ThermoFisher Scientific), etc.

The term “Xkr4” is intended to include fragments, variants (e.g., allelic variants), and derivatives thereof. Representative human Xkr4 cDNA and human Xkr4 protein sequences are well-known in the art and are publicly available from the National Center for Biotechnology Information (NCBI). For example, human Xkr4 (NP_443130.1) is encodable by the transcript (NM_052898.2). Nucleic acid and polypeptide sequences of Xkr4 orthologs in organisms other than humans are well-known and include, for example, chimpanzee Xkr4 (NM_001033036.1 and NP_001028208.1), dog Xkr4 (XM_846336.5 and XP_851429.2), cattle Xkr4 (XM 002692650.4 and XP_002692696.2), mouse Xkr4 (NM_001011874.1 and NP_001011874.1), rat Xkr4 (NM_001011971.1 and NP_001011971.1), tropical clawed frog Xkr4 (NM_001032307.1 and NP_001027478.1), and zebrafish Xkr4 (NM_001012258.1 and NP_001012258.1; NM_001077752.1 and NP_001071220.1). Representative sequences of Xkr4 orthologs are presented below in Table 2A.

Reagents useful for detecting Xkr4 and cleaved forms thereof are known in the art. For example, Xkr4 can be detected using antibodies CABT-BL3812 (Creative Diagnostics), TA324416 and TA351963 (Origene), NBP1-93567 (Novus Biologicals), Cat #PA5-51272 and Cat #PA5-55225 (ThermoFisher Scientific), etc. Some of these Xkr8 antibodies bind to a C-terminal portion of Xkr8, such as TA324416 (Origene).

The term “Xkr3” is intended to include fragments, variants (e.g., allelic variants), and derivatives thereof. Representative human Xkr3 cDNA and human Xkr3 protein sequences are well-known in the art and are publicly available from the National Center for Biotechnology Information (NCBI). For example, human Xkr3 (NP_001305180.1) is encodable by the transcript (NM_001318251.1). Nucleic acid and polypeptide sequences of Xkr3 orthologs in organisms other than humans are well-known. Representative sequences of Xkr3 orthologs are presented below in Table 2A.

Reagents useful for detecting Xkr3 and cleaved forms thereof are known in the art. For example, Xkr8 can be detected using antibodies AP54583PU-N and TA351961 (Origene), ABIN955597 and ABIN1537293 (antibodies-online.com), etc.

The term “serine protease” refers to enzymes that cleave peptide bonds in proteins, in which serine serves as the nucleophilic amino acid at the active site. They are found ubiquitously in both eukaryotes and prokaryotes. Over one third of all known proteolytic enzymes are serine proteases. In some embodiments, the serine protease is a granzyme (e.g., granzyme B).

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “subject” refers to any organism having an immune system, such as an animal, mammal or human. In some embodiments, the subject is healthy. In some embodiments, the subject is afflicted with a disease. The term “subject” is interchangeable with “patient.”

The term “T cell” includes CD4+ T cells and CD8+ T cells. The term T cell also includes both T helper 1 type T cells and T helper 2 type T cells. Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least PCT Publ. WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an peptide antigen-major histocompatibility complex (pMHC) complex. In some cases, an antigen-binding portion or fragment of a TCR may contain only a portion of the structural domains of a full-length or intact TCR, but yet is able to bind the peptide epitope, such as a pMHC complex, to which the full TCR binds. In some cases, an antigen-binding portion contains the variable domains of a TCR, such as variable α chain and variable β chain of a TCR, sufficient to form a binding site for binding to a specific pMHC complex. Generally, the variable chains of a TCR contain complementarity determining regions (CDRs) involved in recognition of the peptide, MHC and/or pMHC complex.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a composition effective for producing some desired therapeutic effect in at least a sub-population of cells in an animal at a reasonable benefit/risk ratio applicable to any medical treatment. Toxicity and therapeutic efficacy of a composition may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the composition. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the composition. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the composition. In some embodiments, response in a desired indicator, such as a T cell immune response, in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in an undesired indicator, such as a viral load, may be achieved.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

“Treating” a disease in a subject or “treating” a subject having a disease refers to subjecting the subject to a pharmaceutical treatment, e.g., the administration of a composition, such that at least one symptom of the disease is decreased or prevented from worsening.

“Vector” refers to a nucleic acid molecule capable of transporting another nucleic acid to which it has been linked. In some embodiments, a vector is an episome, i.e., a nucleic acid capable of extra-chromosomal replication. In some embodiments, a vector is capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Reporters of Phospholipid Scrambling

In certain aspects, provided herein are reporters of phospholipid scrambling.

In some embodiments, the reporter of phospholipid scrambling comprises a scramblase comprising a serine protease cleavage site and/or a caspase cleavage site that activates the scramblase upon cleavage by the serine protease and/or the caspase. In some embodiments, the activated scramblase is capable of promoting the translocation of phosphatidylserine (PS) to the outer leaflet of a cell membrane lipid bi-layer, such as at the cell surface. Such scramblases include, but are not limited to, apoptosis-mediated scrambles, such as members of Xkr family (e.g., Xkr4, Xkr8, Xkr9, and Xkr3). In some embodiments, the scramblase is a human apoptosis-mediated scramblase. For example, the scramblase may be one selected from Table 1A. Apoptosis-mediated scramblases natively comprise a caspase cleavage site. In some embodiments, the native caspase cleavage site is used in the reporter. In some embodiments, the native caspase cleavage site is replaced with a cleavage site of another protease, such as a serine protease like a granzyme or another caspase. In some embodiments, a cleavage site of a protease, such as a serine protease like a granzyme or a caspase, is introduced C-terminal to the native caspase cleavage site position and the native caspase cleavage site position is either maintained in native form or mutated to no longer function as a caspase cleavage site. In some embodiments, more than one protease cleavage site is present in the reporter of phospholipid scrambling.

As described above, structure-function relationships between scramblase activation and scramblase cleavage sites are well-known, as well as the sequences of serine protease and caspase cleavage sites. For example, GzB substrates include those containing P4 to P1 amino acids Ile/Val, Glu/Met/Gln, Pro/Xaa, with an aspartic acid N-terminal to the proteolytic cleavage. Non-charged amino acids are preferred at P1, and Ser, Ala, or Gly are preferred at P2. In certain embodiments, the serine protease or caspase cleavage site comprises (e.g., consists of) an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity with a cleavage site, such as selected from a sequence shown in Table 1A or Table 1B. In certain embodiments, the serine protease or caspase cleavage site comprises (e.g., consists of) an amino acid sequence set forth in Table 1A or Table 1B. In some embodiments, GzB is the serine protease and the cleavage sequence used is one that is cleaved by GzB, but not by caspases, e.g., VGPD (Choi and Mitchison (2013) PNAS 110:6488-6493. In some embodiments, other GzB cleavage sequences are used, e.g., IETD (SEQ ID NO:6) as described in Casciola-Rosen et al. (2007) J. Biol. Chem. 282:4545-4552.

In some embodiments, once activated by serine protease- and/or caspase cleavage site-mediated cleavage, the cleaved scramblase is capable of promoting the translocation of phosphatidylserine (PS) to the outer leaflet of cell membrane lipid bi-layer. The exposed phosphatidylserine (PS) may be detected by an assay such as those described herein (e.g., Annexin-V beads and/or column). Generally, the reporter provides a detectable signal, such as promoting the translocation of phosphatidylserine (PS) to the outer leaflet of cell membrane lipid bi-layer, after serine protease- and/or caspase cleavage site-mediated cleavage of the reporter. This allows for the isolation of cells that have been recognized by a CTL and received GzB.

In certain embodiments, the reporters of granzyme B activity comprises (e.g., consists of) an amino acid sequence having at least 80%, 85%, 90%, 95%, 98%, or 99% identify with SEQ ID NO: 2 or 6. In certain embodiments, the reporter of phospholipid scrambling comprises (e.g., consists of) an amino acid sequence set forth in SEQ ID NO: 2 or 6.

In certain embodiments, the reporters of serine protease or caspase cleavage site activity described herein may be used independently or in combination with other alternative serine protease or caspase cleavage site reporters that serve the purpose of allowing for the detection of serine protease or caspase cleavage site activity in target cells that have been productively recognized by a cytotoxic T lymphocyte (CTL). For example, the reporters of serine protease or caspase cleavage site activity described herein may be used in combination with the GzB-activated IFP reporter comprising a N-fragment (N-IFP) and a C-fragment (C-IFP), functionally separated by the GzB cleavage site, as described in PCT Publ. WO 2018/227091. Additional alternative serine protease or caspase cleavage site reporters that may be used in combination with the reporters described herein include but are not limited to those described in PCT Publ. WO 2018/227091 and Kamiyama et al. (2016) Nat. Commun. 7:11046.

In certain embodiments, the reporters of phospholipid scrambling described herein may be used in combination with reporters that may be used to isolate target cells recognized by CTLs but are independent of phospholipid scrambling, e.g., a caspase-activatable fluorescent reagent, such as CellEvent™.

The alternative reporters may be used to identify and/or isolate target cells recognized by CTLs concurrently or sequentially. For example, target cells may be enriched with the reporters of phospholipid scrambling activity described herein with an Annexin-V bead/column first, and the target cells recognized by CTLs may be further sorted or isolated from the enriched cells based on the detectable signal of another reporter, such as by FACS or affinity purification.

TABLE 2A
Xkr8
Xkr9
Xkr4
Xkr3
Human Xkr8 (hXkr8)
Human Xkr9 (hXkr9)
Human Xkr4 (hXkr4)
Human Xkr3 (hKxr3)
Human XKR8 mRNA sequence; NM_018053.4; CDS: 98-1285 (SEQ ID NO: 9)

| | |
|---|---|
| 1 | gagggctgcg cccacctcct tcctgcctcg gcaaccccgg gccctgaggg caggccccaa |
| 61 | ccgcggagga gcaggagagg gcggaggccg gcgggccatg ccctggtcgt cccgcggcgc |
| 121 | cctccttcgg gacctggtcc tgggcgtgct gggcaccgcc gccttcctgc tcgacctggg |
| 181 | caccgacctg tgggccgccg tccagtatgc gctcggcggc cgctacctgt gggcggcgct |
| 241 | ggtgctggcg ctgctgggcc tggcctccgt ggcgctgcag ctcttcagct ggctctggct |
| 301 | gcgcgctgac cctgccggcc tgcacgggtc gcagcccccg cgccgctgcc tggcgctgct |
| 361 | gcatctcctg cagctgggtt acctgtacag gtgcgtgcag gagctgcggc aggggctgct |
| 421 | ggtgtggcag caggaggagc cctctgagtt tgacttggcc tacgccgact tcctcgccct |
| 481 | ggacatcagc atgctgcggc tcttcgagac cttcttggag acggcaccac agctcacgct |
| 541 | ggtgctggcc atcatgctgc agagtggccg ggctgagtac taccagtggg ttggcatctg |
| 601 | cacatccttc ctgggcatct cgtgggcact gctcgactac caccgggcct tgcgcacctg |
| 661 | cctcccctcc aagccgctcc tgggcctggg ctcctccgtg atctacttcc tgtggaacct |
| 721 | gctgctgctg tggccccgag tcctggctgt ggccctgttc tcagccctct tccccagcta |
| 781 | tgtggccctg cacttcctgg gcctgtggct ggtactgctg ctctgggtct ggcttcaggg |
| 841 | cacagacttc atgccggacc ccagctccga gtggctgtac cgggtgacgg tggccaccat |
| 901 | cctctatttc tcctggttca acgtggctga gggccgcacc cgaggccggg ccatcatcca |
| 961 | cttcgccttc ctcctgagtg acagcattct cctggtggcc acctgggtga ctcatagctc |
| 1021 | ctggctgccc agcgggattc cactgcagct gtggctgcct gtgggatgcg gctgcttctt |
| 1081 | tctgggcctg gctctgcggc ttgtgtacta ccactggctg caccctagct gctgctggaa |
| 1141 | gcccgaccct gaccaggtag acggggcccg gagtctgctt tctccagagg ggtatcagct |
| 1201 | gcctcagaac aggcgcatga cccatttagc acagaagttt ttccccaagg ctaaggatga |
| 1261 | ggctgcttcg ccagtgaagg gataggtgaa cggcgtcctt tgaagcagga tcagacccag |
| 1321 | ccagcagaga tggagagtga ctctgttggc agaaggcagg cgaggataag ctaacgatgc |
| 1381 | tgctgtggcc tctatgcact cagcaagagc gggacgcctg tgctgggccg ggcaccaggg |
| 1441 | atggtgctga gtcgggcaga ggcctccttt caaggagttc acagtgaaca agatgagaag |
| 1501 | ggctgggccc tggagggtca agagccccaa ttatgtacaa gacactttgg gaggaaagaa |
| 1561 | gactaccttt tccccctgcc attggtatag ctggtgcccc aaaacttcca cctccctccc |
| 1621 | tggctacctc taaaatgact ggtataggtg ctgccccacc ccttagctcc cctatcctgg |
| 1681 | gctaggaggc cacaggggct gtcctctaga attcttcctt ccctccccca caccattcat |
| 1741 | tcaattcatg aaacaaatct ttgccaagag cagtttatgt gccaggaaca tcattctgtc |
| 1801 | cttgcaacct ggaacaagac cagctaccag cctagcttca tccgctactt gcaccaacca |
| 1861 | gtcccgggtt agatcccaaa tgctagaagc cagggatgcc caactctggg tggccccagt |
| 1921 | cagaacctct gggatctcag tgaagctggc ctggcctctg ctcctgctct caaggggctg |
| 1981 | cttttcaacc aagagccttg tgagcctggt ctgagccttg cacagccact gagtattttt |
| 2041 | tttgccttag ccagtgtacc tcctacctca gtctatgtga gaggaagaga atgtgtgtgc |
| 2101 | ctgtgggtct ctacaagtga cagatgtgtt gttttcaaca gtattattag gttatgaata |
| 2161 | aagcctcatg aaatcctc |

Human XKR8 amino acid sequence; NP_060523.2 (SEQ ID NO: 10)

| | |
|---|---|
| 1 | mpwssrgall rdlvlgvlgt aaflldlgtd lwaavqyalg grylwaalvl allglasval |
| 61 | qlfswlwlra dpaglhgsqp prrclallhl lqlgylyrcv qelrqgllvw qqeepsefdl |
| 121 | ayadflaldi smlrlfetfl etapqltlvl aimlqsgrae yyqwvgicts flgiswalld |
| 181 | yhralrtclp skpllglgss viyflwnlll lwprvlaval fsalfpsyva lhflglwlvl |
| 241 | llwvwlqgtd fmpdpssewl yrvtvatily fswfnvaegr trgraiihfa fllsdsillv |
| 301 | atwvthsswl psgiplqlwl pvgcgcfflg lalrlvyyhw lphsccwkpd pdqvdgarsl |
| 361 | lspegyqlpq nrrmthlaqk ffpkakdeaa spvkg |

Mouse XKR8 mRNA sequence; NM_201368.1; CDS: 82-1287 (SEQ ID NO: 11)

| | |
|---|---|
| 1 | gacgactgcc ccgccccctt cctgccggac tagcggggcg ggagggcagg tccgcggttg |
| 61 | tgtggttgct tggagaggat catgcctctg tccgtgcacc accatgtggc cttagacgtg |
| 121 | gtcgtaggcc tggtgagtat cttgtctttc ctgctggatc tggtcgctga cctgtgggcc |
| 181 | gttgtccagt acgtgctcct tggccgttat ctgtgggccg cgctggtact ggtcctgctg |
| 241 | ggccaagctt cggtgctgct gacgctcttc agctggctct ggctgacagc tgatcccacc |
| 301 | gagctgcacc attcgcagct ctcgcgtcct ttcctggctc tgctgcacct gctgcagctc |
| 361 | ggctacctgt ataggtgttt gcacggaatg catcaagggc tgtccatgtg ctaccaggag |
| 421 | atgccatccg agtgtgacct ggcctacgca gactttctct ccctggacat cagcatgctg |
| 481 | aagcttttcg agagcttcct ggaggcgacg ccacagctca cactggtgct ggcaattgta |
| 541 | ttgcagaatg gccaggcgga atactaccag tggtttggca tcagctcatc ctttcttggc |
| 601 | atctcgtggg cactgctgga ttaccatcgg tctctgcgta cctgtcttcc ctccaagcca |
| 661 | cgcctgggcc ggagttcctc tgctatctac ttcctgtgga acctgctgct gctggggccc |
| 721 | agaatctgtg ccatcgcctt gttctcagct gtcttcccct actatgtggc cctgcatttc |
| 781 | ttcagcctgt ggctggtact tttgttctgg atctggcttc aaggcacaaa ttttatgcct |
| 841 | gactccaaag gtgagtggct gtaccgggtg acaatggccc tcatcctcta tttctcctgg |
| 901 | ttcaacgtgt ctgggggccg cactcgaggc cgggccgtca tccacctgat cttcatcttc |
| 961 | agtgacagtg ttctgctggt caccacctcc tgggtgacac acggcacctg gctgcccagt |
| 1021 | gggatctcat tgctgatgtg ggtgacaata ggaggagcct gcttcttcct gggactggct |
| 1081 | ttgcgtgtga tctactacct ctggctgcac cctagctgca gctgggaccc tgacctcgtg |
| 1141 | gatgggaccc taggactcct ttctccccat cgtcctccta agctgattta taacaggcgt |
| 1201 | gccaccctgt tagcagagaa cttcttcgcc aaggccaaag ctcgggctgt cctgacagag |
| 1261 | gaggtgcagc tgaatggagt cctctgaggc agggtctgat tcagccagtg aggaagataa |
| 1321 | tgcgagtggg gccttgcaag ggacaaggcg ggccagtcat gtgcaagcca ttttttttct |
| 1381 | tctgaagccg atggaactgc tgtcagcaaa cactcggttg tttgttgttc tcacctctca |
| 1441 | ggtgattggt ggcgtcctgg ctcctggttc cctagcccgc tctagatgac acaagattct |
| 1501 | gggagaactc ttccctaccc catcccatcc attcacttca accaacaaat gctaaaggca |
| 1561 | ctttatgttc tcggaacacc atcctggctt ctgaactgcc tgccactcta gcttctttcc |
| 1621 | ctgcccacct ggacagatcc tgggtagact cctaaacagt gaggccaggt atgtccctcc |
| 1681 | agtgtcctga tgctcaggcc acctttatac caagtgcctt atggacctgt ggtctaggcc |
| 1741 | atgtgatgcc cagtaagtat tttcattctc ctacctcagt ctatgtggaa gaacatatat |
| 1801 | gcatgtgttt aacagtatta aagcctcatg agattctcca gaccagtatg taccactaag |
| 1861 | tgtagtctat caccctttac agacacgtag aaggcgcctg gaacccctta aaactgacac |
| 1921 | agacccctgg catacaaatg tgggcatagg tttgacttaa ttttgcttcc caagacgcag |
| 1981 | gggctagtga gcccgagccg gttgatcatt cggctagcag aactcatggg cagatgctag |
| 2041 | tgtattcttt tagcagctcc gtactgagcc taaagaggac ttgaggatgg ggatggcagg |
| 2101 | tttgaggggc tggatggaag gtaaaggatt gggggttctt tttgggtgag aggtgcagtg |
| 2161 | gcttctggga tgtggtcaat agctccgtgg aggtggcgtg ttctgctctc ggaggtttgt |
| 2221 | ggtcttgttg ggaaaaggga acaggagaga ggctccaggg gcagaagaaa aggttccagg |
| 2281 | tcccagtgct gggacccaga tagttctagc agtcattcat ttatttgtgt ggacgtgaaa |
| 2341 | taacctgtga cccaaacaag caccaagtac tgaaagaaaa ccagatggag aggtgagagg |
| 2401 | gaggatgtat gttgtgggtg gaagttgcag ctttataaaa aaccattggg gaggacccct |
| 2461 | ctgagaaact gaggcataga ctgtaagcta cttcagcagt gactgcagca tggagtctgc |
| 2521 | gtggtttgtt ggagaaggaa tctgcgaatg ctgttccctg tggcacagca accccactgt |
| 2581 | aagaggactg tggggtgcgg ttggctcaca gccaaggagg ctgcagagat gcaggtgggg |
| 2641 | gcctggaaga ggctctggga gaaggtactt cttatactaa aaggtacagg ctgactatgg |
| 2701 | acagaaagga cctaatttcc agacctgaat tttacagacc aggaaaagga gccaaagtgg |
| 2761 | ttgttgatgt taaaagggtc tgaaaaacag tcaccacctc cgtgttcact ctcatggaaa |
| 2821 | aacggatgta atcacaccag aaggtgtcat cctctaaaca gatgccccca caggtacaca |
| 2881 | cctgaaatca ctgttactct catttatgaa aatggtaaga tagggatgag ccagtgtgac |
| 2941 | acacctacca gtctgggcaa ggacatcagg agttcagact cctcagtgac aatgtcagag |
| 3001 | gccagcttgg gctacatgag accctgtctc caacaaaatg aaattatttt atttatttat |
| 3061 | ttatttggct ttttgagacg gggtttctct gtgtagccct ggctgtcctg gaactcactc |
| 3121 | tgtagaccag gctatcctca aactcagaaa tctgcctgcc tctgcctccc aagtgctggg |
| 3181 | attaaaggca tgcgccacca cgcctggcac attttttttt taaattaaaa aaagaaagac |
| 3241 | gttactaccc tgctcttgtt ttgtgacaca caatctggtc tgagaggacc ctgagcacat |
| 3301 | cttccttcct tcaacactac cgtgctaagt tcttaaaatc tcggacttaa aaccaggtta |
| 3361 | gtgacattac ccgtagttag gatgtttggt ttgttgggga ttggttctaa tgctctgtct |
| 3421 | taattcggct cccagaatca cacgggaatc tgctctgcta aaggaagcct gtcactagtt |
| 3481 | ggctgtgatt gggaaataaa gttgcccagg gctggctggg caggaaagag gcgggacttt |
| 3541 | taggttgtga gggcaaggaa ccccggggag ttggaagcag agggatttca ctgcgcagtt |
| 3601 | gggtctgggg cagcagagat gaaatgatga cttagcaagt cgactcaggg aggttagggg |
| 3661 | ggtagaatgt atgctagtcg cacggagggt tagacacgtc cagccactga gctagtcaga |
| 3721 | gcatatcaaa gttagatggt gtgtgtctct cattcacaaa tcccgggaac acttggccag |
| 3781 | ccgggagtca ggggtctaag cactacaggg tttggaaacc agccaacact agaatctgca |
| 3841 | cttgtgactg agcaggggta cggacaacag ctaacagtct acttgagctg cactgcggct |
| 3901 | cagaagatca cttcccggag aaaattcacc ttggagtccg acatatctca cctttggaag |
| 3961 | ctagaaacaa cttctaattt ccttcactgg aacaatgggt aaaaagccct cttgtaagct |
| 4021 | agtgggggcc aatcagacca aatgtggcag aatgtagaac acctggttgg tgggacggga |
| 4081 | agtcaggatt tattgggttg cggcttaatt aatgctcagc acagactgac tcctccttgg |
| 4141 | taacgttcag cacactcgac agctctgaaa tccattccat ttctatacct taaaaagcag |
| 4201 | tgtattttag aaacaattca aataaacatt tctctcgc |

Mouse XKR8 amino acid sequence: NP_958756.1; (SEQ ID NO: 12)

| | |
|---|---|
| 1 | mplsvhhhva ldvvvglvsi lsflldlvad lwavvqyvll grylwaalvl vllgqasvll |
| 61 | qlfswlwlta dptelhhsql srpflallhl 1qlgylyrcl hgmhqglsmc yqempsecdl |
| 121 | ayadflsldi smlklfesfl eatpqltlvl aivlqngqae yyqwfgisss flgiswalld |
| 181 | yhrslrtclp skprlgrsss aiyflwnlll lgpricaial fsavfpyyva lhffslwlvl |
| 241 | lfwiwlqgtn fmpdskgewl yrvtmalily fswfnvsggr trgravihli fifsdsvllv |
| 301 | ttswythgtw lpsgisllmw vtiggacffl glalrviyyl wlhpscswdp dlvdgtlgl1 |
| 361 | sphrppkliy nrratllaen ffakakarav lteevqlngv l |

Rat XKR8 mRNA sequence; NM_001012099.1; CDS: 886-2085; (SEQ ID NO: 13)

| | |
|---|---|
| 1 | tgtgaggacg tctgccgaag ggagcatgtg tgcgccatac agcacgtgga gttcgacact |
| 61 | tacgccacct gcttgcatgg tcttggtgcc aacctggtac ctggtttcct gctcatactg |
| 121 | actctgctga cgagcctaca cgtattggag gtgctatgac tgtaggcact gccagcctac |
| 181 | cctcttactt ggttcgtctt tctccctggt aaaactgggc aacattaccc aatggagaga |
| 241 | gagggagaty aattttgcca tcagtctgtg gagagtaagg tcggatggga catttggatt |
| 301 | caccagagag ggcgctaaga agcacatttc ttctgagttt tatgttttat ccacagagct |
| 361 | tgtttgcggt acatgtcttg gtgcattatt ccctttaata caaacatcaa actatcatgc |
| 421 | acttgatcgc cacagtaaag tgaacccgca ggaagatggg ccctggagag tctgtgcttt |
| 481 | tgagtccctg ctcaaggtct aaaactggga acccacgtgg tctgcaaaat cccttggtac |
| 541 | ttttaaataa aagacttttc tgatttggtt tcgcaacagt gcaaccgtga gggatcacag |
| 601 | ctgcgaccca gacactagtc ttgtggccac tcttgttaac tagagcctca aaaggcagaa |
| 661 | tccaaaccag tagaggcagg gctcaagaca gggagggctg ggggcggggt ctgggcggtg |
| 721 | ggaccgccta gggggcggag tcgtggactc gctcctcccc ggacggggcg agatggggaa |
| 781 | gttccgccca gcagcccggc ctctgggagg actgccccac ccccttcctg ccggactagc |
| 841 | cgggctggag ggcagatccg cggttgtgag gttgcctgga gggccatgcc tctgtccgtg |
| 901 | cacccccaag tggccttaga cgtggtcata ggtctggtga gtaccttgtc tttcctgttg |
| 961 | gacctggtcg ccgacctgtg ggccgtcgtc cagtacgtgc tcgttggccg ttacctgtgg |
| 1021 | gccgcgctgg tagtggtgct gctgggccaa gcctcggtgc tgctgcagct cttcagctgg |
| 1081 | ctctggctga cagctgaccc caccgagctg caccagttgc agccctcgcg tcgtttcctg |
| 1141 | gctctgctgc acctgctgca gctcggctac ctgtataggt gcctgcacgg aatgcggcag |
| 1201 | ggactgtcca tgtgctgcca ggaggtaccg tctgaatgtg acctggccta tgctgacttc |
| 1261 | ctctccctgg acatcagcat gctgcggctt tttgagagct tcttggaggc gaccccacag |
| 1321 | ctcacgctgg tgctggccat cgtgttgcag agtggaaatg ccgaatacta ccagtggttt |
| 1381 | ggcatcagct catcctttct gggcatctcg tgggcattgc tggactacca tcggtccttg |
| 1441 | cgcacctgcc tcccctccaa gccgcgcctg ggctggtgct cctctgcggt ctacttcctg |
| 1501 | tggaacctgc tgctgttggg gccccggatc tgtgccatcg ccacgttctc ggtcgtcttt |
| 1561 | ccctactgct tggccctgca tttcctcagc ctgtggctgg tgctgttgta ctgggtctgg |
| 1621 | cttcaagaca cgaagtttat gccaaactct aatggcgagt ggctataccg ggtgacggtg |
| 1681 | gcgctcatcc tttatttctc ctggttcaat gtgtctgggg gtcgcactcg aggccgggcc |
| 1741 | actatccacc tgggcttcat cctcagtgac agtgttctgc ttgtcaccac ctcctgggtg |
| 1801 | acagatagta cctggttgcc cggtggggtc ttattgtggg cggctttagg cggcgcctgc |
| 1861 | ttctccctgg gactggtttt gcgtatgatc tactacctcc ggctgcaccc tagctgcagc |
| 1921 | tcggaacccg actttgtgga tcggacccta agactcctcc ctcccgagcg tcctccaaag |
| 1981 | ctgatttata acaggcgtgc cactcggtta gcacagaact tctttgccaa gctcaaaacc |
| 2041 | caggccgccc tcccacaggc ggtacagctg aacggagtcc tctgaggcag ggtctgattc |
| 2101 | agccagtgag gaagatgagg agagtggggc cttgcaaggg acaagggggc caatcatgtg |
| 2161 | caagccagtt tttttcctct ccaaccgata gagcttccat tcccaaatct tcagttgtta |
| 2221 | ccactttcac ctctcacgtg attggtggcg tcctggttcc tggttcccta gcctgctcta |
| 2281 | gatgacagac tctgggggat gttctcgaga actcttccct aacctatccc atccattcac |
| 2341 | ttcccccaac aaatgcactg atgttctggg agcatcatcc tgacttctga actggctgcc |
| 2401 | accctagctt ctttccctgc ccacctggac aaatcctccg tagactcttg aagagcggag |
| 2461 | ggaggccaga gatgcccctc cagtgtcctg acgttcaggc tcttaggcca ccttacacca |
| 2521 | agtgccttat ggacctgtgg cctaggccat gtgatgccca ccaagtattt ttcattctcc |
| 2581 | tacctcagtc tgtgtgaaag aagaacatgt gtgcatgtgt ttaacagtat taaaacctca |
| 2641 | cgagagtctc caaaaaaaaa aaaaaaaaaa a |

Rat XKR8 amino acid sequence; NP_001012099.1 (SEQ ID NO: 14)

| | |
|---|---|
| 1 | mplsvhpqva ldvviglvst lsflldlvad lwavvqyvlv grylwaalvv vllgqasvll |
| 61 | qlfswlwlta dptelhqlqp srrflallhl 1qlgylyrcl hgmrqglsmc cqevpsecdl |
| 121 | ayadflsldi smlrlfesfl eatpqltlvl aivlqsgnae yyqwfgisss flgiswalld |
| 181 | yhrslrtclp skprlgwcss avyflwnlll lgpricaiat fsvvfpycla lhflslwlvl |
| 241 | lywvwlqdtk fmpnsngewl yrvtvalily fswfnvsggr trgratihlg filsdsvllv |
| 301 | ttswvtdstw lpggvllwaa lggacfslgl vlrmiyylrl hpscswepdf vdgtlrllpp |
| 361 | erppkliynr ratrlaqnff aklktqaalp qavqlngvl |

Human XKR9 transcript variant 1 sequence; NM_001011720.2; CDS: 561-1682
(SEQ ID NO: 15)

| | |
|---|---|
| 1 | agaggtcacg tgacgccgcg cgggctgcgc gggcagtggt gggaaggctg gcgcgaggcg |
| 61 | tgaggtggcg tgaggcgaag ctggaatctg cctctgtcac gggggctggt gcctcacggg |
| 121 | tttgtgtcct agacaggcga gtggatccaa gtgggcgaga gacattttaa tctggaagag |
| 181 | tcttgtgatt tcggagacag tgaagaagaa gtaaaatatt cacaagatga agatttttcc |
| 241 | agaagggact ttgagtcaaa gatggctttt tatatttgac aagtcttgtc atctgtaatg |
| 301 | aagatcattg tgaaacagaa gattgattaa agccttgtaa cattggacct agattagaga |
| 361 | tttagaaaag aaagtcaaaa ttagtcactt tagtgttagt gttcccattt cataatattt |
| 421 | attctttctt ctaaatagat ttagggagta gaaattaaaa ttcaatgcta taccaaaggg |
| 481 | tatactaata tttgtttggc tttttttccc tttttgtgag ggagaaaaaa gtagataacg |
| 541 | aaaagctata gtcattcgta atgaaatata ctaaacagaa ttttatgatg tcagttcttg |
| 601 | gcattataat ctacgtaact gatttaattg tggacatatg ggtatctgtc agatttttcc |
| 661 | atgaaggaca gtatgttttt agtgctttag cgttaagctt tatgcttttt ggaacacttg |
| 721 | tggctcagtg ttttagttat tcttggttca aggctgattt aaagaaagca ggccaagaaa |
| 781 | gtcagcattg ttttcttcta cttcattgct tgcaaggagg agtttttaca aggtattggt |
| 841 | ttgccttaaa aaggggttac catgcagctt ttaaatatga cagcaatact agtaacttcg |
| 901 | tggaagaaca aattgatcta cataaagaag ttatagatag agtgactgat ttgagcatgc |
| 961 | tcagactatt tgagacctac ctggaaggct gcccacaact tattcttcaa ctctacattc |
| 1021 | ttctggagca tggacaagcg aatttcagtc agtatgcggc catcatggtc tcttgctgtg |
| 1081 | ctatttcttg gtcaactgtt gattatcaag tagctttaag aaaatccttg cctgacaaaa |
| 1141 | agcttcttaa tcgattatgt cccaaaatca catatctctt ttacaagttg tttacattat |
| 1201 | tatcgtggat gctgagtgtt gtacttctac tattcttaaa tcttaagatt gctttatttc |
| 1261 | tcttgttatt tctttggttg ttaggtataa tatgggcatt taaaaacaac acccagtttt |
| 1321 | gtacttgtat aagtatggaa ttcttatata ggattgttgt tggattcatt cttatcttta |
| 1381 | cattttttaa tattaaggga cagaatacca agtgtccaat gtcttgttat tatattgtta |
| 1441 | gggtactggg cactttgggg atattgactg tattctgggt ttgccccctc actattttta |
| 1501 | atccagacta ttttatacct atcagtataa ctatagttct tactcttctt cttggaattc |
| 1561 | tttttcttat tctttattat gggagttttc acccaaacag aagtgcagaa acaaaatgtg |
| 1621 | atgaaattga tggaaaacca gttctaagag aatgtagaat gagatatttc ctaatggaat |
| 1681 | aagctattca tttatgatat atattttctt atattttgtt tcattggtta gtaaagaaaa |
| 1741 | tgtgtgttat gtgggtgtgt tgtctcttat ttttgccacc tttaatttga aattagttca |
| 1801 | gtgaaatagg agatacatag tagtatttta tttttaaaat taatttctca tttggttttg |
| 1861 | aagatcttga gtactcagat atctttctac tgcctggtag agctgccatc ttgagcctga |
| 1921 | aatataagaa atggtctggt tttcataatg agaaggctgg aattgagctt ccctcccatt |
| 1981 | ttccttgttc ctgaactaat actactgtac ctgttatgga ggactgcaaa gggaagagaa |
| 2041 | aagcagaaca ctgtattatt ttttccttta ttgtcttcag tgcatatatt tgcagttggg |
| 2101 | gacaggttga gtagaggaaa agggaaagaa gggaaagcag aaaacaaatt tttagcatct |
| 2161 | gctgtgcttt catccatgaa atctccaatt cagtaagtgc aaaagagaat tggtgtgcat |
| 2221 | ctgagaggtc tgacatttca ttatttactt atttcctagc ttttctgaat taatgcactc |
| 2281 | ttaacatata attatattaa tcctatttgt gctagaatag ttgtatctaa atcatatttt |
| 2341 | aaaattattt ttatttttaa aaaattatgg taaaaacata taaaatttac catcttaatc |
| 2401 | actttgagtg tacagttcat cagtgttaac tgtattcacc ttgtgcaaca gatctcaagg |
| 2461 | actttttcac cttgtaaaac taagattctc tatttattga acaaatcccc atttcctcct |
| 2521 | tccccaagtc tctctcaact gaaattataa ttttttgttt ctatgagttt gaatacttta |
| 2581 | gataccttgt tgccatggtt tgaatgtgcc ccccagattt catgtgtgtg aaacttaatc |
| 2641 | tccaaatttg tatgttgatg gcatttggaa gtggtgggga ctttgtttat ttatttattt |
| 2701 | ttaatttttt aattttatat tattattatt attattatac tttaaggttt agggtacatg |
| 2761 | tgcacaatgt gcaggttagt tacatatgta tacatgtgcc atgctggtgt gctgcaccca |
| 2821 | ttaactcgtc atttatcatt aggtatatct cctaaagcta tccctccccc ctccccccac |
| 2881 | cccacaacag tccccagagt gtgatgatcc ccttcctgtg tccatgtgtt ctcattgttc |
| 2941 | agttcccacc tatgagtgag aatatgcagt gtttggtttt ttgttcttgc gatagtttac |
| 3001 | tgagaatgat gatttccagc ttcatccatg tccctacaaa ggacatgaac tcatcatttt |
| 3061 | ttatggctgc atagtattcc atggtgtata tgtgccacat tttcttaatc cagtctattg |
| 3121 | ttgttggaca tttgggttgg ttccaagtct ttgctattgt gaatagtgct gcaataaaca |
| 3181 | tacgtgtgca tgtgtcttta |

Human XKR9 transcript variant 2 sequence; NM_001287258.2; CDS: 1075-1800
(SEQ ID NO: 16)

| | |
|---|---|
| 1 | agaggtcacg tgacgccgcg cgggctgcgc gggcagtggt gggaaggctg gcgcgaggcg |
| 61 | tgaggtggcg tgaggcgaag ctggaatctg cctctgtcac gggggctggt gcctcacggg |
| 121 | tttgtgtcct agacaggcga gtggatccaa gtgggcgaga gacattttaa tctggaagag |
| 181 | tcttgtgatt tcggagacag tgaagaagaa gtaaaatatt cacaagatga agatttttcc |
| 241 | agaagggact ttgagtcaaa gatggctttt tatatttgac aagtcttgtc atctgtaatg |
| 301 | aagatcattg tgaaacagaa gattgattaa agccttgtaa cattggacct agattagaga |
| 361 | tttagaaaag aaagtcaaaa ttagtcactt tagtgttagt gttcccattt cataatattt |
| 421 | attctttctt ctaaatagat ttagggagta gaaattaaaa ttcaatgcta taccaaaggg |
| 481 | tatactaata tttgtttggc tttttttccc tttttgtgag ggagaaaaaa gtagataacg |
| 541 | aaaagctata gtcattcgta atgaaatata ctaaacagaa ttttatgatg tcagttcttg |
| 601 | gcattataat ctacgtaact gatttaattg tggacatatg ggtatctgtc agatttttcc |
| 661 | atgaaggaca gtatgttttt agtgctttag cgttaagctt tatgcttttt ggaacacttg |
| 721 | tggctcagtg ttttagttat tcttggttca aggctgattt aaagaaagca ggccaagaaa |
| 781 | gtcagcattg ttttcttcta cttcattgct tgcaaggagg agtttttaca agggccttgc |
| 841 | tctgtcaccc aggctggcct gcagtggcgc cttcccagct cattgcagcc tccacctcct |
| 901 | tcgttcaaga gattctcctg catcagcttc ctgagtagct gggattacag gtattggttt |
| 961 | gccttaaaaa ggggttacca tccagctttt aaatatgaca gcaatactag taacttcgtg |
| 1021 | gaagaacaaa ttgatctaca taaagaagtt atagatagag tgactgattt gagcatgctc |
| 1081 | agactatttg agacctacct ggaaggctgc ccacaactta ttcttcaact ctacattctt |
| 1141 | ctggagcatg gacaagcgaa tttcagtcag tatgcggcca tcatggtctc ttgctgtgct |
| 1201 | atttcttggt caactgttga ttatcaagta gctttaagaa aatccttgcc tgacaaaaag |
| 1261 | cttcttaatg gattatgtcc caaaatcaca tatctctttt acaagttgtt tacattatta |
| 1321 | tcgtggatgc tgagtgttgt acttctacta ttcttaaatg ttaagattgc tttatttctg |
| 1381 | ttgttatttc tttggttgtt aggtataata tcggcattta aaaacaacac ccagttttgt |
| 1441 | acttgtataa gtatggaatt cttatatagg attgttgttg gattcattct tatctttaca |
| 1501 | ttttttaata ttaagggaca gaataccaag tgtccaatgt cttgttatta tattgttagg |
| 1561 | gtactgggca ctttggggat attgactgta ttctgggttt gccccctcac tatttttaat |
| 1621 | ccagactatt ttatacctat cagtataact atagttctta ctcttcttct tggaattctt |
| 1681 | tttcttattg tttattatgg gagttttcac ccaaacagaa gtgcagaaac aaaatgtgat |
| 1741 | gaaattgatg gaaaaccagt tctaagagaa tgtagaatga gatatttcct aatggaataa |
| 1801 | gctattcatt tatgatatat attttcttat attttgtttc attggttagt aaagaaaatg |
| 1861 | tgtgttatgt gggtgtgttg tctcttattt ttgccacctt taatttgaaa ttagttcagt |
| 1921 | gaaataggag atacatagta gtattttatt tttaaaatta atttctcatt tggttttgaa |
| 1981 | gatcttgagt actcagatat ctttctactg cctggtagag ctgccatctt gagcctgaaa |
| 2041 | tataagaaat ggtctggttt tcataatgag aaggctggaa ttgagcttcc ctcccatttt |
| 2101 | ccttgttcct gaactaatac tactgtacct gttatggagg actgcaaagg gaagagaaaa |
| 2161 | gcagaacact gtattatttt ttcctttatt gtcttcagtg catatatttg cagttgggga |
| 2221 | caggttgagt agaggaaaag ggaaagaagg gaaagcagaa aacaaatttt tagcatctgc |
| 2281 | tgtgctttca tccatgaaat ctccaattca gtaagtgcaa aagagaattg gtgtgcatct |
| 2341 | gagaggtctg acatttcatt atttacttat ttcctagctt ttctgaatta atgcactctt |
| 2401 | aacatataat tatattaatc ctatttgtgc tagaatagtt gtatctaaat catattttaa |
| 2461 | aattattttt atttttaaaa aattatggta aaaacatata aaatttacca tcttaatcac |
| 2521 | tttgagtgta cagttcatca gtgttaactg tattcacctt gtgcaacaga tctcaaggac |
| 2581 | tttttcacct tgtaaaacta agattctcta tttattgaac aaatccccat ttcctccttc |
| 2641 | cccaagtctc tctcaactga aattataatt ttttgtttct atgagtttga atactttaga |
| 2701 | taccttgttg ccatggtttg aatgtgcccc ccagatttca tgtgtgtgaa acttaatctc |
| 2761 | caaatttgta tcttgatggc atttggaagt ggtggggact ttgtttattt atttattttt |
| 2821 | aattttttaa ttttatatta ttattattat tattatactt taaggtttag ggtacatgtg |
| 2881 | cacaatgtgc aggttagtta catatgtata catgtgccat gctggtgtgc tgcacccatt |
| 2941 | aactcgtcat ttatcattag gtatatctcc taaagctatc cctcccccct ccccccaccc |
| 3001 | cacaacagtc cccagagtgt gatgatcccc ttcctgtgtc catgtgttct cattgttcag |
| 3061 | ttcccaccta tgagtgagaa tatgcagtgt ttggtttttt gttcttgcga tagtttactg |
| 3121 | agaatgatga tttccagctt catccatgtc cctacaaagg acatgaactc atcatttttt |
| 3181 | atggctgcat agtattccat ggtgtatatg tgccacattt tcttaatcca gtctattgtt |
| 3241 | gttggacatt tgggttggtt ccaagtcttt gctattgtga atagtgctgc aataaacata |
| 3301 | cgtgtgcatg tgtcttta |

Human XKR9 transcript variant 3 sequence; NM_001287259.2; CDS: 671-1792
(SEQ ID NO: 17)

| | |
|---|---|
| 1 | agaggtcacg tgacgccgcg cgggctgcgc gggcagtggt gggaaggctg gcgcgaggcg |
| 61 | tgaggtggcg tgaggcgaag ctggaatctg cctctgtcac gggggctggt gcctcacggg |
| 121 | tttgtgtcct agacaggcga gtggatccaa gtgggcgaga gacattttaa tctggaagag |
| 181 | tcttgtgatt tcggagacag tgaagaagaa gtaaaatatt cacaagatga agatttttcc |
| 241 | agaagggact ttgagtcaaa gatggctttt tatatttgac aagattcaaa atctagtgca |
| 301 | ttagactttt gaactagctg ttccttcaag ctggaaggct tttccatctc tatgcacatg |
| 361 | gccaatttca ctactcaaat gccaccttct cagtcttgtc atctgtaatg aagatcattg |
| 421 | tgaaacagaa gattgattaa agccttgtaa cattggacct agattagaga tttagaaaag |
| 481 | aaagtcaaaa ttagtcactt tagtgttagt gttcccattt cataatattt attctttctt |
| 541 | ctaaatagat ttagggagta gaaattaaaa ttcaatgcta taccaaaggg tatactaata |
| 601 | tttgtttggc tttttttccc tttttgtgag ggagaaaaaa gtagataacg aaaagctata |
| 661 | gtcattcgta atgaaatata ctaaacagaa ttttatgatg tcagttcttg gcattataat |
| 721 | ctacgtaact gatttaattg tggacatatg ggtatctgtc agatttttcc atgaaggaca |
| 781 | gtatgttttt agtgctttag cgttaagctt tatgcttttt ggaacacttg tggctcagtg |
| 841 | ttttagttat tcttggttca aggctgattt aaagaaagca ggccaagaaa gtcagcattg |
| 901 | ttttcttcta cttcattgct tgcaaggagg agtttttaca aggtattggt ttgccttaaa |
| 961 | aaggggttac catgcagctt ttaaatatga cagcaatact agtaacttcg tcgaagaaca |
| 1021 | aattgatcta cataaagaag ttatagatag agtgactgat ttgagcatgc tcagactatt |
| 1081 | tgagacctac ctggaaggct gcccacaact tattcttcaa ctctacattc ttctggagca |
| 1141 | tggacaagcg aatttcagtc agtatgcggc catcatggtc tcttgctgtg ctatttcttg |
| 1201 | gtcaactgtt gattatcaag tagctttaag aaaatccttg cctgacaaaa agcttcttaa |
| 1261 | tcgattatgt cccaaaatca catatctctt ttacaagttg tttacattat tatcgtggat |
| 1321 | gctgagtgtt gtacttctac tattcttaaa tcttaagatt gctttatttc tgttgttatt |
| 1381 | tctttggttg ttaggtataa tatgggcatt taaaaacaac acccagtttt gtacttgtat |
| 1441 | aagtatggaa ttcttatata ggattgttgt tggattcatt cttatcttta cattttttaa |
| 1501 | tattaaggga cagaatacca agtgtccaat gtcttgttat tatattgtta gggtactggg |
| 1561 | cactttgggg atattgactg tattctgggt ttgccccctc actattttta atccagacta |
| 1621 | ttttatacct atcagtataa ctatagttct tactcttctt cttggaattc tttttcttat |
| 1681 | tgtttattat gggagttttc acccaaacag aagtgcagaa acaaaatgtg atgaaattga |
| 1741 | tggaaaacca gttctaagag aatgtagaat gagatatttc ctaatggaat aagctattca |
| 1801 | tttatgatat atattttctt atattttgtt tcattggtta gtaaagaaaa tgtgtgttat |
| 1861 | gtgggtgtgt tgtctcttat ttttgccacc tttaatttga aattagttca gtgaaatagg |
| 1921 | agatacatag tagtatttta tttttaaaat taatttctca tttggttttg aagatcttga |
| 1981 | gtactcagat atctttctac tgcctggtag agctgccatc ttgagcctga aatataagaa |
| 2041 | atggtctggt tttcataatg agaaggctgg aattgagctt ccctcccatt ttccttgttc |
| 2101 | ctgaactaat actactgtac ctgttatgga ggactgcaaa gggaagagaa aagcagaaca |
| 2161 | ctgtattatt ttttccttta ttgtcttcag tgcatatatt tgcagttggg gacaggttga |
| 2221 | gtagaggaaa agggaaagaa gggaaagcag aaaacaaatt tttagcatct gctgtgcttt |
| 2281 | catccatgaa atctccaatt cagtaagtgc aaaagagaat tggtgtgcat ctgagaggtc |
| 2341 | tgacatttca ttatttactt atttcctagc ttttctgaat taatgcactc ttaacatata |
| 2401 | attatattaa tcctatttgt gctagaatag ttgtatctaa atcatatttt aaaattattt |
| 2461 | ttatttttaa aaaattatgg taaaaacata taaaatttac catcttaatc actttgagtg |
| 2521 | tacagttcat cagtgttaac tgtattcacc ttgtgcaaca gatctcaagg actttttcac |
| 2581 | cttgtaaaac taagattctc tatttattga acaaatcccc atttcctcct tccccaagtc |
| 2641 | tctctcaact gaaattataa ttttttgttt ctatgagttt gaatacttta gataccttgt |
| 2701 | tgccatggtt tgaatgtgcc ccccagattt catgtgtgtg aaacttaatc tccaaatttg |
| 2761 | tatgttgatg gcatttggaa gtggtgggga ctttgtttat ttatttattt ttaatttttt |
| 2821 | aattttatat tattattatt attattatac tttaaggttt agggtacatg tgcacaatgt |
| 2881 | gcaggttagt tacatatgta tacatgtgcc atgctggtgt gctgcaccca ttaactcgtc |
| 2941 | atttatcatt aggtatatct cctaaagcta tccctccccc ctccccccac cccacaacag |
| 3001 | tccccagagt gtgatgatcc ccttcctgtg tccatgtgtt ctcattgttc agttcccacc |
| 3061 | tatgagtgag aatatgcagt gtttggtttt ttgttcttgc gatagtttac tgagaatgat |
| 3121 | gatttccagc ttcatccatg tccctacaaa ggacatgaac tcatcatttt ttatggctgc |
| 3181 | atagtattcc atggtgtata tgtgccacat tttcttaatc cagtctattg ttgttggaca |
| 3241 | tttgggttgg ttccaagtct ttgctattgt gaatagtgct gcaataaaca tacgtgtgca |
| 3301 | tgtgtcttta |

Human XKR9 transcript variant 3 sequence; NM_001287259.2; CDS: 671-1792
(SEQ ID NO: 18)

| | |
|---|---|
| 1 | agaggtcacg tgacgccgcg cgggctgcgc gggcagtggt gggaaggctg gcgcgaggcg |
| 61 | tgaggtggcg tgaggcgaag ctggaatctg cctctgtcac gggggctggt gcctcacggg |
| 121 | tttgtgtcct agacaggcga gtggatccaa gtgggcgaga gacattttaa tctggaagag |
| 181 | tcttgtgatt tcggagacag tgaagaagaa gtaaaatatt cacaagatga agatttttcc |
| 241 | agaagggact ttgagtcaaa gatggctttt tatatttgac aagattcaaa atctagtgca |
| 301 | ttagactttt gaactagctg ttccttcaag ctggaaggct tttccatctc tatgcacatg |
| 361 | gccaatttca ctactcaaat gccaccttct cagtcttgtc atctgtaatg aagatcattg |
| 421 | tgaaacagaa gattgattaa agccttgtaa cattggacct agattagaga tttagaaaag |
| 481 | aaagtcaaaa ttagtcactt tagtgttagt gttcccattt cataatattt attctttctt |
| 541 | ctaaatagat ttagggagta gaaattaaaa ttcaatgcta taccaaaggg tatactaata |
| 601 | tttgtttggc tttttttccc tttttgtgag ggagaaaaaa gtagataacg aaaagctata |
| 661 | gtcattcgta atgaaatata ctaaacagaa ttttatgatg tcagttcttg gcattataat |
| 721 | ctacgtaact gatttaattg tggacatatg ggtatctgtc agatttttcc atgaaggaca |
| 781 | gtatgttttt agtgctttag cgttaagctt tatgcttttt ggaacacttg tggctcagtg |
| 841 | ttttagttat tcttggttca aggctgattt aaagaaagca ggccaagaaa gtcagcattg |
| 901 | ttttcttcta cttcattgct tgcaaggagg agtttttaca aggtattggt ttgccttaaa |
| 961 | aaggggttac catgcagctt ttaaatatga cagcaatact agtaacttcg tcgaagaaca |
| 1021 | aattgatcta cataaagaag ttatagatag agtgactgat ttgagcatgc tcagactatt |
| 1081 | tgagacctac ctggaaggct gcccacaact tattcttcaa ctctacattc ttctggagca |
| 1141 | tggacaagcg aatttcagtc agtatgcggc catcatggtc tcttgctgtg ctatttcttg |
| 1201 | gtcaactgtt gattatcaag tagctttaag aaaatccttg cctgacaaaa agcttcttaa |
| 1261 | tcgattatgt cccaaaatca catatctctt ttacaagttg tttacattat tatcgtggat |
| 1321 | gctgagtgtt gtacttctac tattcttaaa tcttaagatt gctttatttc tgttgttatt |
| 1381 | tctttggttg ttaggtataa tatgggcatt taaaaacaac acccagtttt gtacttgtat |
| 1441 | aagtatggaa ttcttatata ggattgttgt tggattcatt cttatcttta cattttttaa |
| 1501 | tattaaggga cagaatacca agtgtccaat gtcttgttat tatattgtta gggtactggg |
| 1561 | cactttgggg atattgactg tattctgggt ttgccccctc actattttta atccagacta |
| 1621 | ttttatacct atcagtataa ctatagttct tactcttctt cttggaattc tttttcttat |
| 1681 | tgttatgtgg gtgtgttgtc tcttattttt gccaccttta atttgaaatt agttcagtga |
| 1741 | aataggagat acatagtagt attttatttt taaaattaat ttctcatttg gttttgaaga |
| 1801 | tcttgagtac tcagatatct ttctactgcc tggtagagct gccatcttga gcctgaaata |
| 1861 | taagaaatgg tctggttttc ataatgagaa ggctggaatt gagcttccct cccattttcc |
| 1921 | ttgttcctga actaatacta ctgtacctgt tatggaggac tccaaaggga agagaaaagc |
| 1981 | agaacactgt attatttttt cctttattgt cttcagtgca tatatttgca gttggggaca |
| 2041 | ggttgagtag aggaaaaggg aaagaaggga aagcagaaaa caaattttta gcatctgctg |
| 2101 | tgctttcatc catgaaatct ccaattcagt aagtgcaaaa gagaattggt gtgcatctga |
| 2161 | gaggtctgac atttcattat ttacttattt cctagctttt ctgaattaat gcactcttaa |
| 2221 | catataatta tattaatcct atttgtgcta gaatagttgt atctaaatca tattttaaaa |
| 2281 | ttatttttat ttttaaaaaa ttatggtaaa aacatataaa atttaccatc ttaatcactt |
| 2341 | tgagtgtaca gttcatcagt gttaactgta ttcaccttgt gcaacagatc tcaaggactt |
| 2401 | tttcaccttg taaaactaag attctctatt tattgaacaa atccccattt cctccttccc |
| 2461 | caagtctctc tcaactgaaa ttataatttt ttgtttctat gagtttgaat actttagata |
| 2521 | ccttgttgcc atggtttgaa tgtgcccccc agatttcatg tgtgtgaaac ttaatctcca |
| 2581 | aatttgtatg ttgatggcat ttggaagtgg tggggacttt gtttatttat ttatttttaa |
| 2641 | ttttttaatt ttatattatt attattatta ttatacttta aggtttaggg tacatgtgca |
| 2701 | caatgtgcag gttagttaca tatgtataca tgtgccatgc tggtgtgctg cacccattaa |
| 2761 | ctcgtcattt atcattaggt atatctccta aagctatccc tcccccctcc ccccacccca |
| 2821 | caacagtccc cagagtgtga tgatcccctt cctgtgtcca tgtgttctca ttgttcagtt |
| 2881 | cccacctatg agtgagaata tgcagtgttt ggttttttgt tcttgcgata gtttactgag |
| 2941 | aatgatgatt tccagcttca tccatgtccc tacaaaggac atgaactcat cattttttat |
| 3001 | ggctgcatag tattccatgg tgtatatgtg ccacattttc ttaatccagt ctattgttgt |
| 3061 | tggacatttg ggttggttcc aagtctttgc tattgtgaat agtgctgcaa taaacatacg |
| 3121 | tgtgcatgtg tcttta |

Human XKR9 isoform 1 sequence; NP_001274187.1; (SEQ ID NO: 19)

| | |
|---|---|
| 1 | mlrlfetyle gcpqlilqly illehgqanf sqyaaimvsc caiswstvdy qvalrkslpd |
| 61 | kkllnglcpk itylfyklft llswmlsvvl llflnvkial flllflwllg iiwafknntq |
| 121 | fctcismefl yrivvgfili ftffnikgqn tkcpmscyyi vrvlgtlgil tvfwvcplti |
| 181 | fnpdyfipis itivltlllg ilflivyygs fhpnrsaetk cdeidgkpvl recrmryflm |
| 241 | e |

Human XKR9 isoform 2 sequence; NP_001011720.1; NP_001274188.1; and
NP_001274189.1; (SEQ ID NO: 20)

| | |
|---|---|
| 1 | mkytkqnfmm svlgiiiyvt dlivdiwvsv rffhegqyvf salalsfmlf gtlvaqcfsy |
| 61 | swfkadlkka gqesqhcfll lhclqggvft rywfalkrgy haafkydsnt snfveeqidl |
| 121 | hkevidrvtd lsmlrlfety legcpqlilq lyillehgqa nfsqyaaimv sccaiswstv |
| 181 | dyqvalrksl pdkkllnglc pkitylfykl ftllswmlsv vlllflnvki alflllflwl |
| 241 | lgiiwafknn tqfctcisme flyrivvgfi liftffnikg qntkcpmscy yivrvlgtlg |
| 301 | iltvfwvcpl tifnpdyfip isitivltll lgilflivyy gsfhpnrsae tkcdeidgkp |
| 361 | vlrecrmryf lme |

Mouse XKR9 mRNA sequence; NM_001011873.2; CDS: 465-1586; (SEQ ID NO: 21)

| | |
|---|---|
| 1 | gatcctaaag agttagacag tgaagaaata gaactcataa gctgaagatt tccaagaaga |
| 61 | gacattgagt taaagaaggc ttttatattt gtcacaaaca ttgttatctg taatgaagat |
| 121 | cacagcagag gcgaagatac agcaaggcct tcttgtacca cttgatctgg cgtagacatt |
| 181 | tttttttaaa ggaagttaaa gttattcact tttgttttag tgttccaatt tcataatatt |
| 241 | tatttattta tttttcgtac taggcactga atataggagt gtatgaatgt tagataaaca |
| 301 | ctccatcact gaactatatc accatattct tttcactagt tagactcagt gtataaatta |
| 361 | caattcaatg ctaacccaaa agatacacta gtatccattg tggcattttc ccctattttt |
| 421 | gtatctgaaa aggagtaact aggcaatagc cacagtcctt cataatgaaa tataccaagt |
| 481 | gtaattttat gatgtccgtt ttgggcatta taatctatgt aactgattta gttgcagaca |
| 541 | ttgtcctatc tgttaggtac ttccatgatg gacaatatgt tcttggtgtt ttaaccttga |
| 601 | gctttgtgct ttgtggaaca ctcatagtcc attgttttag ctactcatgg ttgaaggctg |
| 661 | acttagagaa agcaggacaa gaaaatgaac gttattttct tctacttcat tgcttgcaag |
| 721 | gaggagtttt cacaaggtat tggtttgcct tgagaacggg ttaccatgtg gttttcaaac |
| 781 | acagcgacag gaagagtaat tttatggagg agcaaacgga tcctcacaaa gaagcaatag |
| 841 | acatggccac cgacttgagc atgctcaggc tgtttgagac ctacctggaa ggctgcccgc |
| 901 | aactcattct ccagctctat gcctttctgg agtgtggcca ggcaaattta agtcagtgca |
| 961 | tggtcatcat ggtttcctgc tgtgctattt cttggtcaac tgttgactat caaatagctt |
| 1021 | taagaaaatc attgcccgat aaaaatcttc tccgaggact ctggcccaaa ctcatgtatc |
| 1081 | tcttttacaa gttgcttacc ttgttatcct ggatgctgag tgttgtactt ctgctgttcg |
| 1141 | tagatgtgag ggttgctttg cttctgctat tatttctttg gatcacaggc ttcatatggg |
| 1201 | catttataaa ccatactcag ttttgtaatt ctgtaagtat ggagttctta tataggattg |
| 1261 | tggttggatt catccttgtg tttacatttt ttaatatcaa ggggcagaat accaaatgcc |
| 1321 | caatgtcttg ttattatact gtaagagtgc taggcaccct gggaatcttg actgtattct |
| 1381 | ggatctaccc tctttctatc tttaactctg actattttat ccctattagt gccaccatag |
| 1441 | ttcttgctct tctccttggg attatttttc ttggtgttta ttatggaaat tttcacccaa |
| 1501 | atagaaatgt agaaccacaa cttgatgaaa ctgatggaaa agcacctcag agagattgta |
| 1561 | gaataagata ttttctaatg gactaacttg tgaattcatg agaaatattt tatttttttt |
| 1621 | gtttcattgc ctagtaaaaa aaatgtctgt catatgtatg tgttgttact tagtttatca |
| 1681 | cctctgtctg aaatgagtta tggcacatgg tgaatgagag catagtaata ttttatggtt |
| 1741 | taaaataatt tcttctttgt gttgctgagg atcaggcctg cacatgctat gtaaatattc |
| 1801 | taccactgag ttgcaccccc agccatctcg ctggttccaa aagtcttgag tgttgagata |
| 1861 | gttgctttct gtctgataga gctgccatgt tgttcctcaa gtggaataaa caatgtggtc |
| 1921 | ccataa |

Mouse XKR9 amino acid sequence; NP_001011873.1 (SEQ ID NO: 22)

| | |
|---|---|
| 1 | mkytkcnfmm svlgiiiyvt dlvadivlsv ryfhdgqyvl gvltlsfvlc gtlivhcfsy |
| 61 | swlkadleka gqeneryfll lhclqggvft rywfalrtgy hvvfkhsdrk snfmeeqtdp |
| 121 | hkeaidmatd lsmlrlfety legcpqlilq lyaflecgqa nlsqcmvimv sccaiswstv |
| 181 | dyqialrksl pdknllrglw pklmylfykl ltllswmlsv vlllfvdvrv alllllflwi |
| 241 | tgfiwafinh tqfcnsvsme flyrivvgfi lvftffnikg qntkcpmscy ytvrvlgtlg |
| 301 | iltvfwiypl sifnsdyfip isativlall lgiiflgvyy gnfhpnrnve pqldetdgka |
| 361 | pqrdcriryf lmd |

Rat XKR9 mRNA sequence: NM_001012229.1; CDS: 472-1593; (SEQ ID NO: 23)

| | |
|---|---|
| 1 | gatcctaaag tgttcgacag tgaagaaata aaactcatat gctgacgact tccaagaagg |
| 61 | gacattgaat taaagaaggc ttttttatat ttgtcacaaa cattggtatc cgtaatgaag |
| 121 | attgtgatgg aggagaagat acagcagggc ctccttgtgc tactgggtct ggagtagaga |
| 181 | ttttttaaaa aagaaagtta aagttattca tttttgtttt agtgctccga tttcatagta |
| 241 | tttatttatt tatttatttt tggtactagg gactgaatat aggaatttat aaatgttaga |
| 301 | taaacactct gtcactgaac tatatcacca tattcttttc tctgagtaga ctcagagagt |
| 361 | agaaattaca attcagtgct aacacaaaag atacactagt atccattgtg gcatttcccc |
| 421 | tgtttttgta tctgaaaaag agtagctagg caagagccac aggccttcat aatgaaatac |
| 481 | accatatgca attttatgat gtcagttttg ggcattataa tctatgtaac tgatttagtt |
| 541 | gcggacattg tcctaactgt taggtacttc tatgacggac aatatgtttt tggtgtttta |
| 601 | accttgagct ttgtgctttg tggaacactc atagtccatt gttttagcta ctcatggttg |
| 661 | aaggacgact taaagaaagc aggaggagaa aatgaacatt attttcttct gcttcattgc |
| 721 | ttgcaaggag gagttttcac aaggtattgg tttgtcctga gaacaggtta ccatgtggtt |
| 781 | ttcaaacaca gccacaggac aagtaatttt atggaggaac aaacagatcc tcacaaagaa |
| 841 | gcaatagaca tggccaccga cttgagcatg ctcagactgt ttgagaccta cctggagggc |
| 901 | tgcccacaac tcatccttca gctctatgcc tttctggagc gtggccaggc aaattttagt |
| 961 | caatacatgg tcatcatggt ttcctgctgt gctatttctt ggtcaactgt cgactatcaa |
| 1021 | atagctttaa gaaaatcatt gcctgataaa aatctcctca gaggattctg gcccaagctc |
| 1081 | acgtatctct tctacaagtt gtttaccttg ttatcctgga tgctgagtgt tgtacttctg |
| 1141 | ctctttgtgg atgtgaggac tgttctgctt ctgctcttat ttctgtggac tgtaggcttc |
| 1201 | atatgggcat ttataaatca cactcagttt tgcaattctc taagtatgga gttcttatac |
| 1261 | aggctggtgg ttggattcat ccttgtgttc acgtttttta atatcaaggg gcagaatacc |
| 1321 | aaatgtccaa tgtcttgcta ttacactgta agggtgcttg gcaccctggg aatcttgact |
| 1381 | gtgttctgga tttaccctct ctctattttt aactctgact attttatccc tatcagtgcc |
| 1441 | accatcgttc tctctcttct atttgggatt atttttcttg gtgtgtatta tggaacttat |
| 1501 | cacccaaata taaatgcagg gacacaacac gacgaacctg atggaaaagc acctcagaga |
| 1561 | gattgtagaa taagatattt tctaatggac taagttgtga atttatgaga aatgtctttt |
| 1621 | ttttttcatt gcctagtaaa gaaaatgtct gtcatatgta catgctgtta cttagtttgt |
| 1681 | cacttctgac ttgaaatgag ttatggtaca tggtgaatga gaagataata ttttaaggat |
| 1741 | taaaataatt tcttctttgt gttgccaagg attaggccct gtgcatgtta tcccaccact |
| 1801 | gagttgcaac cccagccatc tcgctggttt caaaagtctt gagtattgag gtagttacta |
| 1861 | ttccatcaag cgaataaaca gtgaggccca taaaaaaaaa aaaaaaaaa |

Rat XKR9 amino acid sequence; NP_001012229.1 (SEQ ID NO: 24)

| | |
|---|---|
| 1 | mkyticnfmm svlgiiiyvt dlvadivltv ryfydgqyvf gvltlsfvlc gtlivhcfsy |
| 61 | swlkddlkka ggenehyfll lhclqggvft rywfvlrtgy hvvfkhshrt snfmeeqtdp |
| 121 | hkeaidmatd lsmlrlfety legcpqlilq lyaflergqa nfsqymvimv sccaiswstv |
| 181 | dyqialrksl pdknllrgfw pkltylfykl ftllswmlsv vlllfvdvrt vlllllflwt |
| 241 | vgfiwafinh tqfcnslsme flyrlvvgfi lvftffnikg qntkcpmscy ytvrvlgtlg |
| 301 | iltvfwiypl sifnsdyfip isativlsll fgiiflgvyy gtyhpninag tqhdepdgka |
| 361 | pqrdcriryf lmd |

Human XKR4 mRNA sequence; NM_052898.2; CDS: 462-2414; (SEQ ID NO: 25)

| | |
|---|---|
| 1 | atcctctccc tcggagtcag ctggtggagg agaggaagcg ggaggaggga gcgcgcgcga |
| 61 | ggggaggaga ggaatgtgca ggtccgagga gcgccgcggc ggccgctgct gctcctgctg |
| 121 | ctggcggcgg cggcggctcg ggcggcagca gcgaagccgg gacggcgagg agcgcgggcg |
| 181 | gcgggcaggg gcgcgcgcgg ggcgccgcga gcagcttggc tccgcgcagg cagccaggcg |
| 241 | gcgctcctgc cggccccagg cgcgccgcta gcccggccca gcgcccagcc cggcgggcgg |
| 301 | cgggcggcgg cggacggcag gcgagccgac gcaggagcag gaggaggggg agccgcaccg |
| 361 | cctgggaggg aagccggggc gaggcgagga ggtggcggga ggaggagaca gcggggaaag |
| 421 | gtgtcagata aaggagggct ctcctccggt gtggaggcat catggccgct aaatcagacg |
| 481 | ggaggctgaa aatgaagaaa agcagcgacg tggcgttcac cccgctgcag aactcggacc |
| 541 | actcgggctc ggtgcaggga ttggctccag gcttgccgtc ggggtcggga gccgaggacg |
| 601 | aggaggcggc cgggggcggc tgctgcccgg acggcggcgg ctgctcgcgc tgctgctgct |
| 661 | gctgcgccgg gagtggcggc tccgcgggct cgggcggctc cggcggcgtc gccggcccgg |
| 721 | gcggcggcgg ggcgggctcg gctgcgctgt gcctgcgcct gggcagggag cagcggcgct |
| 781 | actcactgtg ggactgcctc tggatcctgg ccgccgtggc cgtgtacttc gcggacgtgg |
| 841 | gcacagacgt ctggctcgcc gtggactact acctgcgcgg ccagcgctgg tggttcgggc |
| 901 | tcacgctctt cttcgtggtg ctcggctctc tgtcggtgca agtgttcagc ttccgctggt |
| 961 | ttgtgcacga tttcagcacc gaggacagcg ccacggccgc tgctgcctcc agctgcccgc |
| 1021 | agcctggagc cgattgcaag acggtggtcg gcggtgggtc tgcagccggg gaaggcgagg |
| 1081 | ctcgtccttc cacgccgcaa aggcaagcat ctaacgccag caagagcaac atcgccgcgg |
| 1141 | ccaacagcgg cagcaacagc agcggggcta cccgggccag tggcaagcac aggtctgcgt |
| 1201 | cctgctcctt ctgcatctgg ctcctgcagt cactcatcca catcttgcag ctcgggcaaa |
| 1261 | tctggagata tttccacaca atatacttag gtattcgaag ccgacagagt ggggagaatg |
| 1321 | acagatggag gttttactgg aaaatggtat atgagtatgc ggatgtgagt atgctgcatt |
| 1381 | tgctagccac ctttctggaa agtgctccac agctggtcct gcagctctgc attatcgtac |
| 1441 | agactcatag cttacaggcc ctccaaggtt tcacagcggc agcttccctc gtgtccctgg |
| 1501 | cctgggcctt ggcctcctac cagaaggccc tccgggactc tcgagatgac aagaagccca |
| 1561 | tcagctacat ggccgtcatc atccagttct gctggcactt cttcaccatc gccgccaggg |
| 1621 | tcatcacgtt tgccctcttt gcctcggttt tccagctgta ctttgggatc ttcatcgtcc |
| 1681 | ttcactggtg catcatgacc ttctggatcg tccactgtga gacagaattc tgtatcacca |
| 1741 | aatgggaaga gattgtgttc gacatggtgg tggggattat ctatatcttc agttggttca |
| 1801 | atgtcaagga aggcaggaca cgctgcaggc tattcattta ctattttgtg atccttttgg |
| 1861 | aaaatacagc cttgagtgcc ctctggtacc tctacaaggc tccccagatt gcagacgcat |
| 1921 | ttgccattcc agcgctgtgt gtggtgttca gcagcttttt aactggcgtt gtttttatgc |
| 1981 | tgatgtatta tgccttcttt catcccaatg gacccagatt cgggcagtca ccaagttgtg |
| 2041 | cttgtgagga cccagccgct gccttcactt tgcccccaga cgtggccaca agcaccctac |
| 2101 | ggtccatctc caacaaccgc agtgttgtca gcgaccgcga tcagaaattc gcagagcggg |
| 2161 | atgggtgtgt acctgtcttt caagtgaggc ccactgcccc atccacccca tcatctcgcc |
| 2221 | caccacggat tgaagaatca gtcattaaaa ttgacttgtt caggaatagg tacccagcat |
| 2281 | gggagagaca tgttttggac cgaagcctcc gaaaggctat tttagctttt gaatgttccc |
| 2341 | catctcctcc aaggctgcag tacaaagatg atgcccttat tcaggagcgg ttggagtacg |
| 2401 | aaaccacttt ataaagcaaa aggagttgca ggacccacaa catccagatg aaggggtgac |
| 2461 | agcagggctg tggccataat gacacttcat cctagagcag ggcagtgagc cgtgaagttc |
| 2521 | ctagtgggac cgtcatcacc attatcattt gatcctgtcg gctgggggcg gctggtctcc |
| 2581 | ttccaaagca gctgcacccg agagtctctg actccacctg aaagaatgac gctggcttaa |
| 2641 | taggactctc cattgctacc aaactcctcc tgcacggtct tgggtgcacc caccagaggg |
| 2701 | tactactatt atggaaaaat tttgcctcca atcattaggg tgtcttgatg gcgttaactg |
| 2761 | atctttccat aaaaatagat tcagtcatac acacatacac acactaacac acataagtta |
| 2821 | caccagtcct ctgtcaaaaa agcttaggtg acttttcttg atgcaaagct ctgattccca |
| 2881 | caggaatata aaaacaaaga aagagggaaa catccctcga gaaaaaaaat agtattgctt |
| 2941 | agaaaagaaa ccattttctc atttggaaat ccataccatg tgtaaattaa ctatccaacg |
| 3001 | gacagcaaac ccaaatgttg tctacacatg tgttagcatt gatggagtgg ttcattttct |
| 3061 | acacatttca ggatttgttt tatattttaa attttcagtt gcgaacatcc tttttgacag |
| 3121 | aaatcctatg cagcccatgt acggctttca acaagaccaa ggagctcaat aacttcatga |
| 3181 | atagtaatca tgattcagta ttcaattgca tgtgaaaatc aaaatgtaac aggtacacaa |
| 3241 | agaggaagtg gggaaaaagg caaaatgaga gtctgattcc caggcatgtg cagcgcccat |
| 3301 | tgggacataa cggcagtgcg gcgcgagcca gaggaatggg ctggaaccgg atctgtttcc |
| 3361 | agacgcagaa tgagtggctc tgtgtgacca taggcagatg ctgactctgg aagactccgt |
| 3421 | gccactcctt tctagtgcca aacaccatcc aaccacagga ctgacgtgga agccccaaac |
| 3481 | aactgagaat gagtggcatg agccccctaa aagcaggcga gagaacgagc aatcaagttc |
| 3541 | tccactgtgt acagactttt cctcccccca atccaaggtc aaagtgatgt gtcttttaga |
| 3601 | ggctttggga cactttttag taagtatgag cagacaaatg caatgaatat gctatgaaaa |
| 3661 | aacccttctg aactgagaga gggcttatca ctatatccag ctaagatttg tatttgaatc |
| 3721 | atctgtaaag tcgcactctt acaacaagct tctgggtttt aaatacctcc gtacagcaag |
| 3781 | taaacgttcc ccgctttctg ttctcagtgt cctcggtcat ggtgcttttc gttgcattaa |
| 3841 | aagtgccggt caaactttga tagtattttt ttatagttgg tgcagagtgg aataactcat |
| 3901 | ggattatttc aatatttttg taataaaaaa tatagggtat acacataggc atcatcacat |
| 3961 | tttttataga cctggaatcg tttaaaatac tttaagcatc ataattactt gggatgtcag |
| 4021 | aaactggtcc acaaattcca tcagcctgcc tcagcagatt gaaaacattt gtctcttgca |
| 4081 | agatcaccct actttgcaag ttggtgcccc caggaacctg gccaggggtg ctatcagaat |
| 4141 | atcaggtgaa gagagaatca gcttaaatag aaagggcttg tcaagactgg ccaatgtttc |
| 4201 | ccaggaaatc aaagatgtaa atgattactt tcatccatcc attataacaa acctgaccac |
| 4261 | agtggaagct gtcttaaact tccttccctg gttttatatt aacccaactg atagattaag |
| 4321 | tattagtcaa accactaaaa aagaaaaaga aaaaagttta acttaattat tcggttattt |
| 4381 | ggatctaatt cacacaaagt agtccagttc tctagccacc acctgtaatg ggtgtgtcat |
| 4441 | ccagagactg tgtccccacg atgacatcca caggaagtaa cagagggctc aacctaggac |
| 4501 | ttcttttggt acaaagcccc aaatcaattt ttttaaaaaa tagacaattt ttataagtag |
| 4561 | acatacttcc tagtactcca tgatttgatc ctccaagcaa gatttccact aaaaaatact |
| 4621 | aatcttttgt tgggatgtgg aaagattacc tagtcaccag taaaggccca ggaaaaggct |
| 4681 | cttcttgtca gcacatggtg aaaacattcc atccccactg gagaaggaaa aaacgatttt |
| 4741 | ggcaaattct tcacttttgt gcagaacctt gagttattag cttcattgtt tccaagacaa |
| 4801 | cttttaactg atgatctttg gaaattgagt ttctcagttg aactgtacct ttgattctat |
| 4861 | gagtaaatca cagattacag tctaatagag tcaatcaatc aacacaaacc caacaggccc |
| 4921 | catcatgctt caatcatgta agttctaagt tatttctcaa cttgatccct cattcaacat |
| 4981 | gttaagagtc agaatgaata ctatgtcaat gaaaaatgat gtactgtgct ttgacttgga |
| 5041 | ggtgagattg gcagtcagga gaatgtaagg aggttgaatt tttcagtgat ttcccaaata |
| 5101 | ctgtaaatac tctgttatcc gacatatttg gagattatga tcttttaatt aggcatgaat |
| 5161 | tcttgttaag gaaagaacat atccatgaty tgatgaatta caacctttca aaagattaca |
| 5221 | agagcaaaac aagagataaa tcatgattta gccttgcttc catgattcag gaagcactac |
| 5281 | actgccatca gactgttgtg gtaataacaa cttttacttg ttttctagat gcacagataa |
| 5341 | cagagagttt aaagtattca gatttaaaga gacatcatca gtgtacaaag aaacaaagtt |
| 5401 | tcatttttgt atttatattt taattctaac atttcctttt caatctgcca ttaaaccctc |
| 5461 | cgcagacagt aactggagaa tcccaaagga aaaaattgga aatgctgggt tccttatctg |
| 5521 | caggctcctt tctgtgtctg agtccacttt gattccattt aagagggaga tctgctctta |
| 5581 | ctcacttttt gcataggatc aggaaatttt ctaaaggaac aacattgtaa tttgttttac |
| 5641 | ttttaaactt gcatttctaa atatgaaacc atgtttaatg aatatatata atgtgtgtgt |
| 5701 | gtgtatctta accatagtga cactttaagt gtttgtgtga aagaaaagga aataattttt |
| 5761 | ccatgtaagt caaagtttag tctcccaaaa tgactatgtc ctttaaatcc tctttgctta |
| 5821 | tttacttaac tacatactgt ctagttcaat agcactgact ttgcagacac ttagttacta |
| 5881 | ctcatttgtg ataaacgctg ttaacccaac aaatataata aattctctta ctgacatggc |
| 5941 | aagaatatat aattcaagta ttagcaaaag ataatctgag gataaaagta aaatgaagta |
| 6001 | ttttatggtt aatttctaaa tgcccaattt attttgctct atgagtaaag gaagtgattg |
| 6061 | cacagaacaa ttaaaagtga atgagaatag ttgaaaactc aatggctgtt ttttaaaaat |
| 6121 | gatatgtgcc ttttaagtgt gtttgtgtac atacatatat gtatatatac gtacctatat |
| 6181 | atgtatgtac acacacacac acacacactt tccaactaaa gtaacagaga tgaaaaggat |
| 6241 | aaagtatata ctgcttttga atgtatataa agtggtatgt tatgcatata aattgtacat |
| 6301 | aaacttttta gaaaagaagc attttcctgc tcctttttca aaaccaaccc aagcttacag |
| 6361 | tccatctata agaccaacac acttacgaac ttcagttgga aatacctaaa tataattcag |
| 6421 | cacttcttag ctcgaatgag ttttatcact tcttaaggat ctcatctttt aaacagctga |
| 6481 | ataaaatagt tctgtgtcac ttcaaagttt ctttctctga acagattgaa ttgagcaaag |
| 6541 | agaacctctt ctgtccttac caggattgtg taaggttaca catttgcttt taaatatacc |
| 6601 | aaatgccgtt gattggaaac aagttctgac acaatgttta gacaagaatc cagagatttt |
| 6661 | ttctaatgaa ccattttcta gactaaatat atgctccctt gcattttcca catatctttg |
| 6721 | ccattagcca ttgctgtttc tatataaagc ttggatgaga tgcctgcatt tttatgtgct |
| 6781 | aaggagaatt ccttaaagcc tttttaaaaa tagctcatac tgtcattcag attatagctc |
| 6841 | agaggatggt tgaagcgcat ggtgaaaaca caggaggact ggggtggtca ttcctataat |
| 6901 | ttcagtgaca gatgcagatc aacgttcctt tgtctcggca atccaatgtc atttttgaaa |
| 6961 | acaatcaaaa agatcgcttg tgtcagcttc tgactcataa cactcctccc acctgatgct |
| 7021 | ccagtgtttc aaaatggcca aggatgggcg attccgctct atcccccatt tctgagactc |
| 7081 | ttgtctggac ctgtaacagg ccgtgaaatg ccctgagcat tcgagtggca tcccttctcc |
| 7141 | tcacataggc acctgggtgg cagcatcaga ccactgaagt tgttgtgttg acatatgtct |
| 7201 | tatctagttg ctgtcctaaa aatgggcatg tggcaagact ctcaatctac agcctcgaca |
| 7261 | gtatcattac tcattctaaa gtaaaactgc agaatatggg tggaattgta taaaaacata |
| 7321 | atgagccatt taattttgct aattgaagca attagtctaa catgcaagca gcctgctctc |
| 7381 | acagcagaga gccacatgga agaagtgcca aatagccatt tgcatttata tatatatatt |
| 7441 | gcaggcagtg acctggcccc caaatgtaaa gcttttgtca accttgaggc ctatattctg |
| 7501 | ctaaacaaga gatgacttaa tgtccttgaa atattttcgt aatatactga cagcctaatg |
| 7561 | tcagaaacga gctgcctaaa tcaagttttg cttttggtta tttcacttcc ccatagactt |
| 7621 | tcttatggtt ccatctccca cattgagagt agctcaccac gatggatggt ttactgcgca |
| 7681 | cctagtgctg gactaagagc tgtatctatg tggtttcatt tagtcctcac tgccatctgt |
| 7741 | gagttaagca tcatttacag atgacaaaat ctgtaaatgg cttagagatg tcaagcaatt |
| 7801 | tgcccaaagg tcccacagct aggaaacagt ggggctgagg gttgagcaca gctttcaaca |
| 7861 | actgcgactt ctgggagccc agtgactctt cccacaaaat ctagtcctga tttggcaagt |
| 7921 | cttcagaaga aacagaatca tggtctgatg atcaaatttt tccaagaaaa ttttatttaa |
| 7981 | aagtcaaaga tgtccttcaa aatgaacagt taaaaatgta aaagtcgatg taaaatggaa |
| 8041 | gtctctatca cctgtaacta aattttacct taactctaac tcatagtagg cagataaatg |
| 8101 | ctattcttcc attccaggca actgtccccc tcctatggct ccactatgta ttcaattaag |
| 8161 | tgataaatat aaattaacct gatgccatgt ctcttgtatt ttatatgtgt atgctgtttt |
| 8221 | catccaatta agcagactga aaaaaaacta aaccccatta cttactttgg cattttgaca |
| 8281 | agatagagag agaggaaaag aaagagggag ggagagaggg agggaaggaa gaaggaagga |
| 8341 | aggaaggaag gaaggaagga aggaaggaag gaaggaagga aggaaggaag gaaggagatt |
| 8401 | taacaagtct ttgaagtgat attttcaaat tataaggtaa ttctgtttca ctgccataat |
| 8461 | ttttccctaa attttattta atatcttgca ggtcacaaac tttaatattt aagaggatta |
| 8521 | ttaaaccact agcttgaaca atcatataag tctaggaacc ttattttagt gttagatgcc |
| 8581 | aataatactg caagtgtcaa ccaaatattt gttgaattga attataaaat aattgatgtg |
| 8641 | ttctttccct tctcacttta gatatagcat gtctgaaggt ctgcaagatg acagagttgt |
| 8701 | aacccattca atgatattgt tgcctagtaa gctgtgtgtg tgttgtttga actgatacta |
| 8761 | aaaaggtagc tgataataaa ccaaaaattt tctcaaccct ggtgtttatt tttaaaaaat |
| 8821 | cttcaatgat caatatgaat gtagtgtatt aaaatacaag taactatctt cctactttga |
| 8881 | tttaagagat ctttatgaat ttatataaaa ttagaagtca ctgattttta taggaaatag |
| 8941 | catgtaaaat aaatctaagt attgctttat cactttattt tatagatgag acaactgaga |
| 9001 | tccaaaaaga acaggtaatt tttgtgatca ggattacaca atacactttt ttttttccct |
| 9061 | gagtcattta ttcaacaagt ttgacctcta caactcattt ggctaggcaa tgcacagtca |
| 9121 | agcacaaaag gaaagttgca ctggaatagc tcatagtctg gctattagca gcacaatcat |
| 9181 | agttttctga cgccagctct tactcttttc tactctacca cactgtttct tctcttctca |
| 9241 | atatctatat ttaattccat attgaagcaa gaaagaaaca cagcttttct aagactatgc |
| 9301 | agtcatgtgt cacttaagga tggggatatg ttctgagata tgcatcgtca ggcaattttg |
| 9361 | tcattgtgtg atggagtgtg cttacacaag cttagatggt agagcctacc atgctcctag |
| 9421 | gctatatggt agagcctatt gtccctaggc tacaaacctg tacagcatgc tactgtaccg |
| 9481 | aatactgtag gcaactgtaa caccatggta agtacttgtg tatttaaata tagaaaagtt |
| 9541 | aacagtaaaa aatatagtat tattgtctta tgggatcgct gtcatatgtg cagtctatta |
| 9601 | ttgaccaaaa tgccattgtg tggcatgtga gccttacaat atacaattaa catatgaaat |
| 9661 | aatgatgatg aacataaagt aacaatacaa atacaaaaaa aaaactagat gactgcttat |
| 9721 | aaagagaaaa gtaattttat aatttgttta tatgactctc caacactaga tatttttaaa |
| 9781 | ttgatatcac aacacacaaa aaaattgaaa tactctcttg gtgcatagta tttgattgaa |
| 9841 | aacaatcatt tttggataaa ctttgaagcg attcttgaga acttatttca agaaaaggca |
| 9901 | tgaaattagg gagactccaa agtgaagagt tttccaatag gtgacttctc tgatttttca |
| 9961 | agaaagcatt cttcactaac tgtatttctc cagcatactg gttatttagg aataacaaat |
| 10021 | ttctggacat aaacatgagc tgtttctcta aagcctttcc tccaatgccc agaagagcag |
| 10081 | cactgtgctg cgtgacaatt tcaggagtca ggagtcagga gtcaggacag tcagccccag |
| 10141 | cttcctgggg aaacccacac tggctttgga cccgattgca ttctctcctg agtgattggc |
| 10201 | ttcccacata tataagcagc agattgttaa agatcactat taacttgtat aactaatttt |
| 10261 | ccttatgtga aataattctg gtcagggaat atataaaccc attggccctc taaggagtag |
| 10321 | aagaaaagag agaagaaagt atattaactt ttatgagtac agaataattc aagttcctta |
| 10381 | gcgagtcaca ttatgcatta ataaaagagt tgacctaata aatgttacaa ggtaccatga |
| 10441 | tctctaggtt catgccacca ttaccacatt ccttactaca attattgcta ttttagtcat |
| 10501 | tggaccagac aaaatgaagc atataattac tgatataata tttgctaagc aaaaatcttg |
| 10561 | tttaacgaaa aaaatcaata ccaaaactaa ttaatcaaaa tattaagcaa atattaccag |
| 10621 | cacagtactg acacaaaatt ttctcttgtg ctagtaattg aagtatgtca tctaccctgt |
| 10681 | tattagaatt tcagaaaata ggccgggcgc agtggctcac gcctgtaatc ccaacacttt |
| 10741 | gggaggctga ggcgggcgga tcacaaggtc aggagatcga gaccatcctg gctaacacag |
| 10801 | tgaaaccccc atctctacta aaactacaaa aaaattagcc aggcatggtg gcgggcgcct |
| 10861 | gtggtcccag ctactcggga ggctgaggca ggagaatggc atgaacccag gaggcagagc |
| 10921 | ttgcagtgag ccaagatcgt gccactgcac tccagcctgg gtgacagagc aagactccgt |
| 10981 | ctcaaaaaaa aaaaaaaaaa aaaaaaagaa tttcagaaaa tataaagttt tatgttttta |
| 11041 | ttatatttcc atctaccaaa ttgttgacct tctcctcctc tccattgctt aatttatatt |
| 11101 | aaaacagatt taatcaaatt attacttaag tactacaaat gttatcagat ggagatgtgg |
| 11161 | ttaagctaat ttaatttacc tattctagtg gcattctggt atggagctgt atcaaatcaa |
| 11221 | cacttttaat tatttcacat taattcatca agaagttcca aaacactact aaatgtgttg |
| 11281 | aaaatatagt ttgagtttct atgattgtaa tcaaaattcc tattttgatc gcacaccagt |
| 11341 | agaacgcatc ttaacaccag cattgccatt gtgagtctag aaaatgagca ctttgtgtgt |
| 11401 | tgagcgctgt tgcattcact tagcaattaa cctttgacct gtggttttct gctgagcccc |
| 11461 | ttgtgatttt ttttattcta ttcaaattgg gagcaataac acaccttaac ataaccaaaa |
| 11521 | aaaggagacc tgtcagctag tgaaagaatt gtcattttat atcattcttt caaaaaatta |
| 11581 | aaatattcaa cttcccttat taacctttct aatgcattgt acataaaaga ggaaatggat |
| 11641 | ttctgaaata tattttgaaa gcctggggtg aaacattttc cacggtctga atcggaagct |
| 11701 | tggggctctg tggaaagaty taaatccctc ctgctgtaag aggagggaag gcagcagtga |
| 11761 | gctgtcactc agaaatacag tcaccactgt cacaaagctg cctattgctg atgctatcga |
| 11821 | ttcccttctt tttctacaga aacatcttgg agcttgtcaa gctttactgg aggtgatttg |
| 11881 | cagttaatta attcaacaga cactttaatc ttgcaaattc ttgacttgta atattgtaac |
| 11941 | caagctcctg caagggaaca ttaatcagtt agtgaaaaag gagcacttcc gttcagccgt |
| 12001 | agtaccatga cgtgcacagg cctgaagaga aatacctctg tgaagtggag cgctagtgaa |
| 12061 | ttcctgctac ctgcttctta tggctcacgc tatgaatatt cacctgcttc atttgttttt |
| 12121 | tccagtaaac gctgttttga aaaaaaagaa aaatattccc gggggcttgc atagctcaga |
| 12181 | gaacggagta ctgggtcgtg gagacttgct ttaaatggat tcaaatccac atgtttggaa |
| 12241 | atgaaaataa tgcactgtca tctgttgaat aattgatctg tctgagtaca gttgctgctt |
| 12301 | ttatttcatt tcttgagact accattgtca gcattgtaat aaccaattta taaaaattga |
| 12361 | gtttttattc agtttcagag gtaaaatctg catgggtgca gctactgaat aatttgattc |
| 12421 | ctgccttctt aggtggtgac attagcagtt ccaaaccgag atccatttct atgtggaatt |
| 12481 | ggctatcctg ttgcttctca ggccctgcaa aaccttggtt acgagctcaa agatcacgaa |
| 12541 | tctgatattc tttttttttt tttttttttt ttttttttga gacagagtct cgctctgtcg |
| 12601 | caggggctgg agtgcagtgg cacaatctcg gctcactgca agctctgcct cccaggttca |
| 12661 | caccatcctt ctgcctcagc cttctgagta ggtgggacta caggcgcctg tcaccacgcc |
| 12721 | cggctaattt ttttgtattt tttagtagag atggggtttc accgtgttag ccagaatggt |
| 12781 | ctcgatctcc tgacctcgtg atctgccctc cttggcctcc caaagtgctg ggattacagg |
| 12841 | cgtgagccac cacacccggc cccgatattc ttaatgacta aattttcaca tagaggtaaa |
| 12901 | cagatcatct cttaatttaa tacatggttc tttctccctt gcttctgggt tttgtttttt |
| 12961 | ttttttcaaa gaaagatttg agctacgaga taagaatgaa gttaccagaa gttatcaggt |
| 13021 | catagtttca gagtatgcaa gagagtcggg ccttcatatg ttcttgtaaa gttttctgtc |
| 13081 | taatcttttg gtataacaat tttaggagtt caccctagat gaaagagtgg aagtcatcag |
| 13141 | atttgtcaat aagcagtcta gaggaaaaat gagaagagga agaagcaggg attctttttc |
| 13201 | ttgtgttttg aagatgtttc tcctcccaaa gctatcacct tggtagttat caccaagatg |
| 13261 | tataatagca agcactactg aatgatcttc ccagttatca gcactagcat cacggcgagt |
| 13321 | cagttttcag aactagctct tggcgcaagc cctgaaataa aatggggaca aaaagtggtc |
| 13381 | taccaccatg tgacttattt tctttttttt tttaatttta ttattattat actttaagtt |
| 13441 | ttagggtaca tgtgcacaac gtgcaggttt gttacatatg tatacatgtg ccatgttggt |
| 13501 | gtgctgtacc tattaactcg tcatttagca tcaggtatat ctcctaatgc tatccctccc |
| 13561 | ccctcccccc accccacaac actccccggt gtgtgatgtt ccccttcctg tgcacgtgac |
| 13621 | ttattttcaa ttgcccagca atgaaaacta acaagttaaa gaaaatgttc attttctgaa |
| 13681 | ccccagagcc cacataggta caaagatact ctgtaatgta caatgaggtg gccaatcgtg |
| 13741 | ggaatatagg agcaataaat agtcctctta agcaaggttc atgggtaaga gttactctag |
| 13801 | caggattggg tgttgggtca gagggtatct attaatgtag aggcccaagt atggtgatga |
| 13861 | agagaaaacc tgtcagtggc tcatccatag tatttgcctt ttcacagagc agagaagttc |
| 13921 | aaaatagtca cagccagtcc ataactataa caacagacat gtccactttg gaaaggctag |
| 13981 | ggcctgacga aagtgggaaa acagagatgt cagtggtgtc atgtctaaga gtgactctgt |
| 14041 | cattagggga acccaccccc tgtgatagtt ctccttgacc actggtccct atgggctctg |
| 14101 | caggagagct tctcgtgggt tctaagataa ggtattccaa ggtattgtaa gttacccttg |
| 14161 | tttgtagaac atgaaccact taaccatccc tccttttaac agcaatgaga ttcagggtta |
| 14221 | ccatggcctt actcatcttc ccattgtaaa tatatcacaa tgtcacaaga gcctctgtgt |
| 14281 | ccaaacacac taaactgggt ttacaagcat tagaatcttt cactcatatt gtgaatctca |
| 14341 | attctgccag tcacctagtc tgtgtatctg ttcccaaact ggaaaaaata attcttgaga |
| 14401 | gaataatttt cagaataatg gaggtggaaa gaaatgaaca gttaagcaat ttttcaacat |
| 14461 | agacaaaacc actggaccat tgatagccct caagctctga ttcttcctcc tgactaagtt |
| 14521 | tcttttcttt ggggggcttt caacatctga attttccaga tgattgcgga accatcgtca |
| 14581 | ctaaaccaaa gtagacaagg agttattaaa aaataaagac tgtccacatg actgcaaata |
| 14641 | tcctgatgaa aagtggccaa gtagatcact caagtggtaa atttggtctt catgatatca |
| 14701 | aacatacgga tatttggaaa agtcgagatg tttgaatcat acagttttcc gtctgggtgt |
| 14761 | ctggtgtttc tggatagaca gactgctccg gtgttgtaag taatggaatt gaactttctt |
| 14821 | gcgccgtaag caattgctgg tcatattctg ctgctaaaag tctctttgtt gtgccaagag |
| 14881 | aaataatgca gaacaaatgt tatttaattt ttatttactt tcagcaaaca catgaatgaa |
| 14941 | agaggtcagg taggctgtcc tgggcattct gggcctggct gcggcacacc ctccttcact |
| 15001 | tcgcccctgc caggcaagaa actttctatt cagtctttgc tatctttcat aaattgtatc |
| 15061 | attgctcttc tgctgttcat atcatcttag ttattcacaa agtctacttg ataaaatggc |
| 15121 | tcaagggaaa tacaagtttc ttaagttttt attcttcaaa tagaagtttt aattttaagc |
| 15181 | attccttatg atatttttta agcctaaaaa ccattcaaat tgcttgacaa aattatttca |
| 15241 | tggtgaattt tataaggttg atagaagtaa aagctatttt tcccaaaaca aacaaaatac |
| 15301 | catacatagt tttttgggtt tggtttgttg atgtcatgcc aatttccaag caccaactgg |
| 15361 | ttaccacaaa catgggaata tttagtgata tctttgtagt catcgttaaa attcctggga |
| 15421 | aaaaaagaaa aagtttacgt caaaggaaaa ttcacctccc acaaggaaag tctgagatgt |
| 15481 | tcatcctgac atttgcgttc ctgattattt gtggacattt cttcattgtg actgtaggaa |
| 15541 | gctgagcttg tttctcctaa tttgacactg ggttggtgag cattgtctca aattttgtgc |
| 15601 | ttgcctcatt tatggtcctg aagcttagca gaaaaacaga caagctattc agaccagttt |
| 15661 | tctttaagag cacttatgtt gcagaacatg atacaaatga ttcaccgtga gcaggcacac |
| 15721 | agagtacgga aaggtattca actatgcaaa gatattgagg ggatttccag agaaaactta |
| 15781 | aatgttttga agatttgtag gtagggtttt gattgtgtca cattctacac tcagtgccaa |
| 15841 | gttagaatgt ctttatgggg aaggcaataa agttacttgt tgggtccttc cttcccttac |
| 15901 | aaacagaatg tttttatgaa atcaaatgga tcctccactt tgtgtagtaa ggacccccca |
| 15961 | ggccccacaa catcatcact gtgagtccta tcgcagatgt gtgtaccagc ccaattcagt |
| 16021 | tttgcttttc tttttcccta agatttttac ttcaccaaat cccatttcaa atctttttac |
| 16081 | cttcatgtta ccaacaggat gtttagttga atcagcaaca aagacgtgac aacctattgt |
| 16141 | cctccacaaa agcatgagtc attttattca gtgatctttg gtagtacgat aatcaatgga |
| 16201 | atttatggtg tcgtagaaaa ccaaaaatcc atgttgaata tagtgactgt cttaaatata |
| 16261 | cttaaatatg ttattctaca aaacaatatc cttttacact atgggatgga ttcctttctg |
| 16321 | gatgcaggga tgggagggtc tatgggtcag tgactgggac aaaggaactg ggaatctctg |
| 16381 | cacaactgag ccctaatccc tggtccatct ctccagcctc agaaactcac cctcagcctc |
| 16441 | attttcccca tatgcaaaag agagatattt atttacctac ctcatagggg tgttgtggag |
| 16501 | attagctaga tttgctaaag tgcttgtagg ttagaaagtg ctgtcattcc tgagaactgg |
| 16561 | cattaacaga agagagctgt gtgcagcacg gaggaagtgg agtctgagga atacaacagc |
| 16621 | aacaactcac caagcagaga atacaatggt tcttcatcac tatataaaac taacactttt |
| 16681 | ccttcaaagg tctatgtata attttcttca atgattagct ttttaatgag acaactcctt |
| 16741 | tcatccagac attcagatgc tttatataag ttggcaattt tcctgttaac caaactgaat |
| 16801 | tttattaaat gtttattaaa atgcacccag aaaacttgtc tcctcctgat gcctgagggg |
| 16861 | tttgcatgcc tgatcccaag ctgcattttt tcagaatgcg tgcatgatgc cccagttctg |
| 16921 | tactcatgat caccaggtgg cgttctgaaa tccactactg gggaaagatt tttaacagat |
| 16981 | attagtgaga ttagagttgg tgtcatttcc attgagtatc ctcttcaccc ctaagatgac |
| 17041 | acatctttac aacacaataa aagaacgtaa agccttattt ccacctgtaa ctcctgaatt |
| 17101 | gattcatttt cacgttataa ctacatttca aatatttcgg agaagttttt acacagggct |
| 17161 | tcagctatat actgatatac atatgcttac atgtgcttag gtgggaattc tactaaagga |
| 17221 | taaaggacac agtgtgaaaa caacatcaga gaatatcctg tacaacttcc ccaaaagtga |
| 17281 | caagttttct tgtacttaaa aatttaatcc tgataagaac taatgtgaaa taacatcatt |
| 17341 | ttggtttata aatatttgta atttttgaga catagaggca atatcatgat ataggaatac |
| 17401 | attcataaaa ctagactagc aaagcagata atgttttcat gatatggctt catgaggcaa |
| 17461 | agttgttgta catcaatatt atcattgtgc ccttatttaa ggattatatt ccattgtgaa |
| 17521 | aaaaatgtgc acactcttaa aaacacaaaa tgggtttcag aaagtttacc ttgagaagtg |
| 17581 | ggtttgaaat catcttgtgc ttggagctga cataagatac gcactcaata tttcccctgc |
| 17641 | tggattctaa aatctaattg gcagtgatat ttcaaagcct taacatttca ttaaactttc |
| 17701 | ttaatatcta atgcatggta tgaagcatga atttaaccta ttgtgctgcc aaaccagact |
| 17761 | tgattcattt tttttaaagt gaagtattgt gtgagtcaaa aaataattgg gactgtcctt |
| 17821 | taatactatg agaatagtaa taatctcttc aggtggttaa ggcaattatc ttttctggac |
| 17881 | ccacttccta gtatcaatac tcccccaacc agaaatgcag cagaatatcc tttttgctat |
| 17941 | aaaggaaaat actgtgtttt tatttgtttt tgcagaagaa aactggtgtt gcctatttgg |
| 18001 | actagatgta ggggcctgga agaaggaagt ggcagattca caggtggggt gaccaggatg |
| 18061 | ggaggaaaat agtggggcga gtatgtcatg gggagatttt gccacaaaga tacaaaacag |
| 18121 | aattgaagtg tgttagagct ggacaaccct ttgaaatgac agagtctaga ttcttcacca |
| 18181 | aacagatgaa aagacaagta gagacaacat gtacttgaga tataagctat acatctcatc |
| 18241 | actggaagaa aggagacttc agcctctttt caaggctttc cagaccacat ggaactctcc |
| 18301 | agagccctcc ttgaaagttt ttagaaaaac taccattttc agcaaagatt catgtgatta |
| 18361 | tgctgctgag gaccagtcat tctgtaaaca tcacatatgt gatgctttgt aaatgtatta |
| 18421 | attgtggtca attttcatgg atatttccca ttaacattgt attccatgaa caagtgatag |
| 18481 | aaaacatatg gaaattctct tttgatcaaa aggagtgtct cccaattagt ttacgtgtgt |
| 18541 | tagtattgct gacatattat tatcatcaca aaattccttt tatatctaga tggtatcaaa |
| 18601 | taagaaaaaa atgcatcatt tggtcaattg cttattgaag atcccagctg aagcctttct |
| 18661 | ttggtaaaga gcgcagaaag agaccatagc tattcttgga tgagaacctt gcctctacta |
| 18721 | aatagtttct gcttttcctc tctgtagcca gacagctcaa tagcctaggg agagtcgatg |
| 18781 | aaggatatgc adattacatt tttcccattc tcagaacada gacagcaacc aatgagccag |
| 18841 | aggtttcttc tctctttgaa accaaatagc acgctgaatt tagggctatg acaaaaatgt |
| 18901 | tgttaaagca agagcaaaat catccttcct atggattctt ttctcagtgt ttacttaatt |
| 18961 | ctttttgcag tttggattgg agtttctagt aatgataatt aatgccattt tacatgatag |
| 19021 | cttcaatgca gaaatggtgt gagcctgagt tacaaatgac atgactaggg atacaaactt |
| 19081 | cgtctgtact aacatcctac caagcagatt ggaaacaaat actactacca ctaatattct |
| 19141 | gatgtaatta ataacatcta atagaaaaat agaaacatcg tgcttagcat gaaaccattg |
| 19201 | cacaatataa acctgctccc aaatggcaag gatttttgct accaatattt gttcttaatt |
| 19261 | ctccagttat tttaagtaaa taagtttcac atctaactac ctcagctact gttgttttat |
| 19321 | ttagaaacat gaaaccatgc actttgtaat caataagtct tttgtttaac atttcaaaag |
| 19381 | gatatttggt gcaaagcaat tttcaaaaat ttgtacatga tatacaccac ccaacctcag |
| 19441 | gaggttgtac ttaattttgt ttgtttgttt ctaaggttgg ttttgggtaa aatcctcatt |
| 19501 | tccactcaac atcaagataa gctgctctat atttgcttaa tttgccttaa acattttgtg |
| 19561 | ctcctttccc tgttcaattt ttttgttttg ttttaaatct atctctgaaa aaaaaatgga |
| 19621 | acaggtggca ggtgaacagc aaatggaaga gaatggacca gtaatttctc agtcccctgt |
| 19681 | tgtcaactat ctgcatgaca ttctgattgt gcaaaaatgc cattcctgtg cttccccctc |
| 19741 | cattacagaa taaggtccga gagaccccac gagtgtgcgt agggaacggt gtagacattt |
| 19801 | cccccagtat gagcacagtg cctggacctg aatgatcatc ttggcagttc ttgtgctttt |
| 19861 | actttgtaaa cattgtacaa atgtatttgg aattttattt gaaatggaga cttaaactag |
| 19921 | ttattaaatt tctttccttc ctgtaaatat atatattcaa attccatgta tccaaacatc |
| 19981 | cctttagcgt tcagattgta agtgtgtctt tattcgcggg aggccactgt cagcaggcag |
| 20041 | tgacccccag tgccctagtt tgaagcacag tgtgtggagt atttgatgta ctacagtacc |
| 20101 | atagttattt tggtctgtta agtaagttgc aatttgtgat gaaatgaagt ggaaagtagt |
| 20161 | acttcataat gaacaaattt ccttggttac atggttttt ttgtaaaact taaagaaaaa |
| 20221 | aaaagaaaac ttgaaatttt a |

Human XKR4 amino acid sequence; NP 443130.1; (SEQ ID NO: 26)

| | |
|---|---|
| 1 | maaksdgrlk mkkssdvaft plansdhsgs vqglapglps gsgaedeeaa gggccpdggg |
| 61 | csrcccccag sggsagsggs ggvagpgggg agsaalclrl greqrryslw dclwilaava |
| 121 | vyfadvgtdv wlavdyylrg qrwwfgltlf fvvlgslsvq vfsfrwfvhd fstedsataa |
| 181 | aasscpqpga dcktvvgggs aagegearps tpqrqasnas ksniaaansg snssgatras |
| 241 | gkhrsascsf ciwllqslih ilqlgqiwry fhtiylgirs rqsgendrwr fywkmvyeya |
| 301 | dvsmlhllat flesapqlvl qlciivqths lqalqgftaa aslvslawal asyqkalrds |
| 361 | rddkkpisym aviiqfcwhf ftiaarvitf alfasvfqly fgifivlhwc imtfwivhce |
| 421 | tefcitkwee ivfdmvvgii yifswfnvke grtrcrlfiy yfvillenta lsalwylyka |
| 481 | pqiadafaip alcvvfssfl tgvvfmlmyy affhpngprf gqspscaced paaaftlppd |
| 541 | vatstlrsis nnrsvvsdrd qkfaerdgcv pvfqvrptap stpssrppri eesvikidlf |
| 601 | rnrypawerh vldrslrkai lafecspspp rlqykddali qerleyettl |

Mouse XKR4 mRNA sequence; NM_001011874.1; CDS: 151-2094; (SEQ ID NO: 27)

| | |
|---|---|
| 1 | gcggcggcgg gcgagcgggc gctggagtag gagctgggga gcggcgcggc cggggaagga |
| 61 | agccagggcg aggcgaggag gtggcgggag gaggagacag cagggacagg tgtcagataa |
| 121 | aggagtgctc tcctccgctg ccgaggcatc atggccgcta agtcagacgg gaggctgaag |
| 181 | atgaagaaga gcagcgacgt ggcgttcacc ccgctgcaga actcggacaa ttcgggctct |
| 241 | gtgcaaggac tggctccagg cttgccgtcg gggtccggag ccgaggacac ggaggcggcc |
| 301 | ggaggcggct gctgcccgga cggcggtggc tgctcgcgct gctgctgctg ctgcgcgggg |
| 361 | agcggcggct cggcgggctc gggcggctcg ggcggcggcg gccggggcag cggggcgggc |
| 421 | tctgcggcgc tgtgcctgcg cctgggcagg gagcagcggc gttactcgct gtgggactgc |
| 481 | ctctggatcc tggccgccgt ggccgtgtac ttcgcggatg tgggaacgga catctggctc |
| 541 | gcggtggact actacctgcg tggccagcgc tggtggtttg ggctcaccct cttcttcgtg |
| 601 | gtgctgggct ccctttctgt gcaagtgttc agcttccgct ggtttgtgca tgatttcagc |
| 661 | accgaggaca gctccacgac caccacctcc agctgccagc agcctggagc agattgcaag |
| 721 | acggtggtca gcagtgggtc tgcagccggg gaaggcgagg ttcgtccttc cacgccgcag |
| 781 | aggcaagcat ccaacgccag caagagcaac atcgccgcca ccaacagcgg cagcaacagc |
| 841 | aacggggcca cccggaccag cggcaaacac aggtctgcgt cctgctcctt ttgcatctgg |
| 901 | ctcctgcagt cactcatcca catcttgcag cttgggcaaa tctggaggta tttgcacaca |
| 961 | atatacttag gtatccggag ccggcagagt ggggagagcg gcaggtggcg gttttactgg |
| 1021 | aagatggtgt acgagtatgc agatgtgagc atgctgcatc tgctagccac ttttctggaa |
| 1081 | agtgctccac aattggtcct gcagctctgc attattgtac agactcacag cttacaggcc |
| 1141 | ctccaaggtt tcacagcagc agcctccctt gtgtccttgg cttgggccct agcctcctac |
| 1201 | cagaaggctc ttcgggactc ccgagatgac aaaaagccca tcagctacat ggctgtcatc |
| 1261 | attcagttct gctggcattt cttcaccatc gctgccaggg tcatcacatt cgccctcttt |
| 1321 | gcctcggttt tccagctgta ttttgggata tttattgtcc tccattggtg catcatgact |
| 1381 | ttctggattg tccactgtga gacagaattc tgtatcacca aatgggaaga gattgtgttt |
| 1441 | gacatggtgg tgggcatcat ctacatcttc agttggttca atgtcaagga aggcaggaca |
| 1501 | cgctgcaggc tgttcattta ctattttgta atccttttgg aaaatacagc cttgagtgca |
| 1561 | ctctggtacc tctacaaagc tccccagatt gcagatgcat ttgccatccc tgcattgtgc |
| 1621 | gtggttttca gcagcttttt aacaggtgtt gtttttatgc tgatgtacta tgccttcttt |
| 1681 | catcccaatg ggcccagatt tgggcaatca ccaagttgtg cttgtgatga tccagccact |
| 1741 | gccttctctc tgcctccaga agtagccaca agcacactac ggtccatctc caacaaccgc |
| 1801 | agtgttgcca gtgaccgtga tcagaaattt gcagagcggg atggatgtgt acctgtgttt |
| 1861 | caagtgagac caactgcacc acccacccca tcatctcgac caccacggat tgaagaatca |
| 1921 | gtcattaaaa ttgacctgtt caggaataga tatccagcat gggagagaca tgtgttagat |
| 1981 | cgaagcctga gaaaggccat tttagccttt gaatgttccc catctcctcc aaggctgcag |
| 2041 | tacaaggatg atgcccttat tcaggagagg ctggaatatg aaaccacttt ataaaataca |
| 2101 | aggagccgca atgtccacat gaaggggtaa cagcagggct gtggcaataa tgacacctta |
| 2161 | tccaagagta gggcagcgag ctgtatgttc ttagttgtgg tatggtttga tcttccatca |
| 2221 | gctgactgcc tgctgctggt gtctattcaa gccagcagtg ctgagagtct cttacactgt |
| 2281 | cagcttaata tgactgttgc tacaaactcc tccagcagag atttggggca cattcactgg |
| 2341 | aggataacat tattgtgaaa aatgttgcct ctaatcatta gggtattttg atgggtttta |
| 2401 | ctaagttttg cataaatata ttcacacacc accataccac ccctcaatca aaggagttaa |
| 2461 | ggtggggatg gagagatgac tcattagtta agagcactga ctgctcttgc aaaggaccca |
| 2521 | ggcttgagta gttcactgca actctaattc cagaagatct aatgtccatt tttggcctcc |
| 2581 | tcaagcactg cacacacatg gtgcatagac atatatgcag gcaaaatacc catacacata |
| 2641 | gcataaaaat aaatctcaaa gaaaaaaagc ttaggtgatt tccttgatgc aaagctcaca |
| 2701 | acatactcca ggaagaaagc agcatacttg ggacaattat ataaactgtt ctctcctttg |
| 2761 | caaaccagta gcatcaatga agtggacagc aagactcaag tgtttacact cgtactaact |
| 2821 | agctttgatg ggatgattct ttttctacat atttcaggat ttgtttttac ttttaggttt |
| 2881 | tgcagatgag aacattcttc atgacagaaa tcctatgcag cacttatatg gcttttgatg |
| 2941 | agaccaagga gctcaatatc tgtaatgtaa attaaatgct aatcataatt cagtattcag |
| 3001 | ttgcaaaaat acaatatata aaaagagtct ttggggaagg gacagagtga gattcagatt |
| 3061 | ctcaggtgtg tgcatcttat attggaatgc acccacagag ccacaggaga ggaacaggga |
| 3121 | ctatttcaag gtctgtgttc atgtctgttt ccagaactgt ttccaggtgc agaatgacat |
| 3181 | gggtcagcag gtatgattcc ggaaaccacg tgccacatct ttcgagtgcc aaattttgtc |
| 3241 | caattacaga actgatatgg aatccccaaa atctgagaat aagtggtttc ccaaaacaga |
| 3301 | caaaagaaga ataatcaggt tccctgctgt gtacagactt accctcttcc catccaaggt |
| 3361 | caaaatgatg tgtctactag agactttggg acacaattta gcaagtgaga gcatacagat |
| 3421 | gcaatgtgta tgccattaaa aatactgcct ggactgcttg agggcttacc actccatcag |
| 3481 | ctaagatttg tatttgaatc atctgtaaat tcgtgctctt acaagcttct gagttttaaa |
| 3541 | tacctccaca cagcaagtaa acattcccgc tttctgtttt cggtgtcctt ggtcatggtg |
| 3601 | ctttttgttg cattaaaagt gccggtcaaa ctttaaaaaa aaaaaaaaaa aa |

Mouse XKR4 amino acid sequence: NP_001011874.1 (SEQ ID NO: 28)

| | |
|---|---|
| 1 | maaksdgrlk mkkssdvaft plansdnsgs vqglapglps gsgaedteaa gggccpdggg |
| 61 | csrcccccag sggsagsggs ggggrgsgag saalclrlgr eqrryslwdc lwilaavavy |
| 121 | fadvgtdiwl avdyylrgqr wwfgltlffv vlgslsvqvf sfrwfvhdfs tedsstttts |
| 241 | scqqpgadck tvvssgsaag egevrpstpq rqasnasksn iaatnsgsns ngatrtsgkh |
| 181 | rsascsfciw llqslihilq lgqiwrylht iylgirsrqs gesgrwrfyw kmvyeyadvs |
| 301 | mlhllatfle sapqlvlqlc iivqthslqa lqgftaaasl vslawalasy qkalrdsrdd |
| 361 | kkpisymavi iqfcwhffti aarvitfalf asvfqlyfgi fivlhwcimt fwivhcetef |
| 421 | citkweeivf dmvvgiiyif swfnvkegrt rcrlfiyyfv illentalsa lwylykapqi |
| 481 | adafaipalc vvfssfltgv vfmlmyyaff hpngprfgqs pscacddpat afslppevat |
| 541 | stlrsisnnr svasdrdqkf aerdgcvpvf qvrptapptp ssrppriees vikidlfrnr |
| 601 | ypawerhvld rslrkailaf ecspspprlq ykddaliqer leyettl |

Rat XKR4 mRNA sequence; NM_001011971.1; CDS: 164-2107; (SEQ ID NO: 29)

| | |
|---|---|
| 1 | atgggtagag ccccagggcc ttcgcatttc tccaggctgg ggtttgccag tacagcatcc |
| 61 | ctgaggctgc cctctcctta tcccgagggc ccgccctctg ctgccggctt tgctttaggt |
| 121 | gttccagccc tacaggtcct ctgccaccca ggatctccaa agcatggcac gcccaccacc |
| 181 | gctgctagta cagaagccca gcttcctagt tgaagcgtgc tgttcaccct cgccggcaac |
| 241 | acacctagca ccgtaccaca cccaaccagg tgcccgaact cccagtacaa tacaaagaga |
| 301 | cctgctcttc cccatccctc gccgctgcca cgcccgctcg agtccacggc cccctgccct |
| 361 | cggcggtggc ccaacacaga gactccaaca cgcggcgcgc tctgcccacc ccatcccccc |
| 421 | cagcgtcaag gaaatccacc caacgttttc cgaaatccca cgagcccggg cctccgactg |
| 481 | ctgtgctgct gccctcggcg tccagcactg gccagcccgg cacccccacc cgccgctccc |
| 541 | ctcgatctcg ctcgctgtgg actactacct gctcggccag cgctggtggt ttgggctcac |
| 601 | cctgttcttc gtggttctgg gctcgctctc tgtgcaagtg ttcagcttcc ggtggtttgt |
| 661 | gcacgatttc agcaccgagg acagcgccac gaccaccgcc tccacctgcc agcagcctgg |
| 721 | agcggattgc aagaccgtgg tcagcagtgg gtctgcagcc ggggaaggcg aggctcgtcc |
| 781 | ttccacgccg cagaggcaag catccaacgc cagcaagagc aacatcgccg ccaccaacag |
| 841 | cggaagcaac agcaacgggg ccaccaggac cagcggcaaa cacaggtctg cgtcctgctc |
| 901 | cttctgcatc tggctcctgc agtcactcat ccacatcttg cagctcgggc aagtctggag |
| 961 | gtatttgcac acaatatact taggtatccg gagccggcag agcggggaga gcagtaggtg |
| 1021 | gcggttttac tggaagatgg tgtacgagta tgcagatgtg agcatgctgc acctgctggc |
| 1081 | cacctttctg gaaagtgcgc cacaactggt cctgcagctc tgcataattg tacagactca |
| 1141 | cagcttacag gccctccaag gttttacagc agcagcctcc cttgtgtcct tggcttgggc |
| 1201 | cctagcctcc taccagaagg ctcttcggga ctcccgagat gacaaaaagc ctatcagcta |
| 1261 | catggctgtc atcatccagt tctgctggca tttcttcacc attgctgcca gggtcatcac |
| 1321 | attcgccctc tttgcctcgg ttttccagct gtattttggg atattcattg tcctccactg |
| 1381 | gtgcatcatg accttctgga ttgtccactg tgagacagaa ttctgtatca ccaaatggga |
| 1441 | agagattgtg tttgacatgg tggtgggtat catctacatc ttcagttggt tcaatgtcaa |
| 1501 | ggaaggcagg acacgctgca ggctgttcat ttactatttt gtaatccttt tggaaaatac |
| 1561 | agccttgagt gcactctggt acctctacaa agctccccag attgcggatg catttgccat |
| 1621 | ccctgcattg tgcgtggttt tcagcagctt tttaacaggt gtcgttttta tgctgatgta |
| 1681 | ctatgccttc ttccatccca atgggcccag atttgggcag tcaccaagtt gtgcttgtga |
| 1741 | cgaccctgcc actgccttct ctatgcctcc agaagtagcc acaagcacac tacggtccat |
| 1801 | ctctaacaac cgcagtgttg ccagtgaccg tgatcagaaa tttgcagagc gggatggatg |
| 1861 | tgtacctgtg tttcaggtga gaccaactgc accacctact ccatcatctc gaccaccgcg |
| 1921 | gattgaagaa tcagtcatta aaattgacct gttcaggaat agatatccag catgggagag |
| 1981 | acatgtgttg gaccgaagcc tgagaaaggc cattttagcc tttgaatgtt ccccatctcc |
| 2041 | tccaaggctg cagtacaaag acgatgccct tattcaggag aggctggaat atgaaaccac |
| 2101 | tttataaaac acaaagaacc gtaatgtcca tataaagggg taacagcagg gctgaggcaa |
| 2161 | taatgacacc ttatccaaga gtagggcaat gagctatatg ttcttagtcc aaacattgtc |
| 2221 | acggtatggt ttgatcttcc atcagctgac tgcctgctgc cggtgagcat tcaagccagt |
| 2281 | agtgctgaga gtttcttact ccgctgaaag gggcgatgtc agcttagtat gactgttgct |
| 2341 | acaaattcct ccagcacagg cttggggcac attcactgga ggataacatt attgtgagga |
| 2401 | aatgttgcct ctaatcatta gggtatttta atggagttta ctaatctttg cataaatatg |
| 2461 | ttcataccac caccaccacc acccctctat caaaggagtt aaggtggagc tggagagatg |
| 2521 | actcagtagt taagagcact catttgatag ttcactacaa caggcactgc actcacatgg |
| 2581 | gactgctctt gcaaagaacc ctctaattcc agaatatcca tgcacagaca tatatgcagg |
| 2641 | caggcttgag ccccagcatc atgcccattt ttggcctcct caaaataccc atacacataa |
| 2701 | aataaaaata aatctccaaa aacaaaacaa aacaaaaaca aaaaaaagtt taggtgattt |
| 2761 | ccttgatgca aagctcacaa cagactccaa gaagaaagca acatgcttgg aatgacccta |
| 2821 | gaaaccattc tctcctttgc aaaccagtag catcaatgac aaaacctgtg cagtggacag |
| 2881 | caagactcaa gtgtttacac tgatactagc atcgatggga tgattctttt tctacgcatt |
| 2941 | tcaggatttg ttttttactt ttaagttttg cagatgagaa cattctttat gacagaaatc |
| 3001 | ctatgcagca catgtatggc ttttgaagag accaaggagc tcaatattca tccgtgatgt |
| 3061 | aaattaaatg ctaatcatga ttcagtattc aattgcaaaa ataaaattta tatacaaaga |
| 3121 | gccatggcgg gagggacaga atgagaatca gattctcagg tgtgtgcatc tcctattgaa |
| 3181 | atacacccac aaagccacgg tcgagaaaaa gggactgttt ccaggtctgt ttctaggtgc |
| 3241 | aggatgagca cgggtcagca ggtgtgattc cggaaaccac atgccacacc tttctagtgc |
| 3301 | caaacttcgt tcaatcacag aactgatacg gtattccccc agactgagaa taagtggtgt |
| 3361 | cccaaaacag acaaggacag aataatcagg ttcttggctg tatacagact taccctcttc |
| 3421 | ccatccaagg tcaaagcgat gtgtctacta gagactttgg gacacctttt agcaagcgag |
| 3481 | tgcatacaga tgcaatgtgt atgctatcaa aaataaaaac tgcctggact gcttgagggc |
| 3541 | ttaccactcc atcagctaag atttgtatgt gaatcatctg taaagttgtg cttttacaag |
| 3601 | cttctgagtt ttaaatacct ccatacagca agtaaacatt cccgctttct gttcttggtg |
| 3661 | tcattggtca tggtgctttt tgttgcatta aaagtgccgg tcaaacttta aaaaaaaaaa |
| 3721 | aaaaaaa |

Rat XKR4 amino acid sequence: NP_001011971.1 (SEQ ID NO: 30)

| | |
|---|---|
| 1 | marpppllvq kpsflveacc spspathlap yhtqpgartp stiqrdllfp iprrcharss |
| 61 | prppalgggp tqrlqhaars ahpippsvke ihptfseipr arasdccaaa lgvqhwparh |
| 121 | phpplpsisl avdyyllgqr wwfgltlffv vlgslsvqvf sfrwfvhdfs tedsatttas |
| 181 | tcqqpgadck tvvssgsaag egearpstpq rqasnasksn iaatnsgsns ngatrtsgkh |
| 241 | rsascsfciw llqslihilq lgqvwrylht iylgirsrqs gessrwrfyw kmvyeyadvs |
| 301 | mlhllatfle sapqlvlqlc iivqthslqa lqgftaaasl vslawalasy qkalrdsrdd |
| 361 | kkpisymavi iqfcwhffti aarvitfalf asvfqlyfgi fivlhwcimt fwivhcetef |
| 421 | citkweeivf dmvvgiiyif swfnvkegrt rcrlfiyyfv illentalsa lwylykapqi |
| 481 | adafaipalc vvfssfltgv vfmlmyyaff hpngprfgqs pscacddpat afsmppevat |
| 541 | stlrsisnnr svasdrdqkf aerdgcvpvf qvrptapptp ssrppriees vikidlfrnr |
| 601 | ypawerhvld rslrkailaf ecspspprlq ykddaliqer leyettl |

Human XKR3 nucleic acid sequence; NM_001318251.1: CDS: 107-1486

| | |
|---|---|
| 1 | cttttgaaat tctaaattct gatgcagaac gtatcagtga aactccctcc cactgtctct |
| 61 | tgtattagca tcaaggaagc gagaaaaaat aagcagcacc ctgagaatgg agacagtgtt |
| 121 | tgaagagatg gatgaagaaa gcacaggagg agtttcatct tcgaaagaag aaatagtcct |
| 181 | tggccagaga ctccatctaa gctttccttt tagcattatc ttctcaactg ttctctactg |
| 241 | tggtgaggtt gcctttggtt tatacatgtt tgaaatttat cgaaaagcta atgacacatt |
| 301 | ctggatgtca tttaccatca gctttattat tgtgggggca attttggatc aaattatcct |
| 361 | gatgtttttc aacaaagact tgaggagaaa taaggctgca ttactttttt ggcacattct |
| 421 | tcttttagga cctattgtga ggtgtttgca caccattaga aattaccaca aatggttgaa |
| 481 | aaatcttaaa caggagaagg aagagactca agttagcatc acaaagagaa acacgatgct |
| 541 | ggaaagggag attgcattct caatccggga taatttcatg cagcagaagg ctttcaagta |
| 601 | catgtcagtg attcaggctt ttctcggttc tgttccacaa ttaattttgc agatgtatat |
| 661 | cagtctcact atacgagaat ggcctttgaa tagagcattg ctgatgacat tttccctgtt |
| 721 | atcagttact tatggggcca ttcgctgcaa tatactggcc atccagatca gcaatgatga |
| 781 | tactaccatt aagctaccgc cgatagaatt cttctgtgtc gtgatgtggc gttttttgga |
| 841 | ggttatctca cgtgtagtga ctctggcatt tttcattgca tctctgaaac tgaagagcct |
| 901 | acccgttttg ttaatcatat attttgtatc attgttggca ccgtggctgg agttttggaa |
| 961 | aagtggagct catcttcctg gcaacaaaga aaataattcc aatatggtgg gtacagtact |
| 1021 | gatgcttttc ttgatcacac tgctatatgc tgccatcaac ttctcctgct ggtcagcagt |
| 1081 | gaaactgcag ttgtcagaty acaaaataat tgacgggaga cagaggtggg gccatagaat |
| 1141 | cctacactac agctttcagt ttttagaaaa tgtgataatg atattggtat ttaggttctt |
| 1201 | tggagggaaa actttgctga attgttgtga ctcattaatt gccgtgcagc tcatcataag |
| 1261 | ctacctattg gccactggct ttatgctcct cttctatcag tatttgtacc catggcagtc |
| 1321 | aggcaaagtg ttgccaggac gtactgaaaa tcagccagaa gcaccgtact attatgtaaa |
| 1381 | catcgagaaa actgaaaaga ataaaaataa gcagctgagg aattactgtc actcctgcaa |
| 1441 | tagggttgga tatttttcaa tcagaaaaag tatgacatgt tcataaaata tacatatata |
| 1501 | ctttcacaga acaatgagta aagatgctga atgtgacttg ttaagaggct cttaaattta |
| 1561 | aaaaatatac acagcaaaat cttggaagtg gtttctaata aaattcattt atgttctcct |
| 1621 | gtgaacgtgc cttagtaatt tttgttttct taactataat tatacaattc attaaataaa |
| 1681 | acaaaataaa aaaaaaaaaa aaaaaaaa |

Human XKR3 amino acid sequence; NM_001305180.1

| | |
|---|---|
| 1 | metvfeemde estggvsssk eeivlgqrlh lsfpfsiifs tvlycgevaf glymfeiyrk |
| 61 | andtfwmsft isfiivgail dqiilmffnk dlrrnkaall fwhilllgpi vrclhtirny |
| 121 | hkwlknlkqe keetqvsitk rntmlereia fsirdnfmqq kafkymsviq aflgsvpqli |
| 181 | lqmyisltir ewplnrallm tfsllsvtyg aircnilaiq isnddttikl ppieffcvvm |
| 241 | wrflevisrv vtlaffiasl klkslpvlli iyfvsllapw lefwksgahl pgnkennsnm |
| 301 | vgtvlmlfli tllyaainfs cwsavklqls ddkiidgrqr wghrilhysf qflenvimil |
| 361 | vfrffggktl lnccdsliav qliisyllat gfmllfyqy1 ypwqsgkvlp grtenqpeap |
| 421 | yyyvniekte knknkqlrny chsenrvgyf sirksmtcs |

TABLE 2B
YW1: hXKR8 GZMB reporter gene DNA sequence (SEQ ID NO: 1)
ATGCCCTGGAGTAGTCGCGGGGCTCTCCTGCGGGACCTTGTGCTGGGAGTACTC
GGGACAGCGGCGTTCCTGTTGGACCTCGGAACTGACTTGTGGGCCGCCGTCCAG
TACGCACTTGGTGGAAGGTACCTTTGGGCGGCGCTGGTCCTGGCCCTCTTGGGG
CTGGCAAGCGTCGCTCTCCAGCTCTTTAGCTGGCTGTGGCTTCGCGCAGATCCC
GCTGGGCTGCATGGGTCCCAGCCGCCAAGGAGATGCCTGGCTCTGCTCCATCTT
CTCCAGCTCGGGTATCTTTACAGATGCGTACAAGAGTTGCGCCAGGGCCTTCTT
GTTTGGCAACAAGAGGAACCAAGTGAGTTCGACCTCGCCTATGCGGATTTCCTT
GCGTTGGATATCTCCATGCTTCGGCTCTTCGAAACATTCCTTGAGACCGCGCCA
CAATTGACCCTTGTACTTGCAATCATGCTGCAATCTGGACGAGCAGAATACTAC
CAATGGGTGGGAATCTGCACATCCTTCCTGGGCATCAGTTGGGCCCTCCTTGAT
TATCATCGCGCCTTGAGAACTTGTTTGCCAAGCAAACCATTGTTGGGCCTCGGA
TCCTCTGTTATTTATTTTCTCTGGAATCTGCTGCTTTTGTGGCCGCGAGTACTCG
CTGTTGCGCTTTTTTCCGCGTTGTTCCCTTCCTACGTCGCGCTCCATTTTCTCGGC
CTGTGGCTGGTTCTGCTGTTGTGGGTTTGGCTGCAAGGGACGGACTTTATGCCA
GACCCGTCCAGTGAGTGGCTTTACCGGGTTACAGTTGCGACCATACTTTATTTC
TCCTGGTTTAATGTCGCAGAGGGACGAACTCGCGGGAGAGCCATAATCCACTTC
GCATTCCTCCTCTCAGATTCAATACTCCTGGTCGCCACCTGGGTAACACACTCA
TCATGGCTCCCAAGTGGGATACCTTTGCAATTGTGGTTGCCGGTTGGCTGCGGG
TGTTTCTTCCTGGGTCTCGCTCTTAGACTTGTCTATTATCATTGGCTGCACCCGA
GTTGCTGCTGGAAGCCTGACCCGGTGGGACCTGATTTTGGTAGAGAATTCGCGC
GGTCCTTGCTCTCCCCAGAAGGCTACCAGTTGCCCCAAAATAGACGCATGACTC
ACCTTGCCCAGAAGTTCTTTCCCAAAGCCAAGGACGAGGCAGCTTCTCCTGTCA
AGGGGTAG
hXKR8 GZMB (YW1) reporter protein sequence (SEQ ID NO: 2)
MPWSSRGALLRDLVLGVLGTAAFLLDLGTDLWAAVQYALGGRYLWAALVLALL
GLASVALQLFSWLWLRADPAGLHGSQPPRRCLALLHLLQLGYLYRCVQELRQGLL
VWQQEEPSEFDLAYADFLALDISMLRLFETFLETAPQLTLVLAIMLQSGRAEYYQW
VGICTSFLGISWALLDYHRALRTCLPSKPLLGLGSSVIYFLWNLLLLWPRVLAVALF
SALFPSYVALHFLGLWLVLLLWVWLQGTDFMPDPSSEWLYRVTVATILYFSWFNV
AEGRTRGRAIIHFAFLLSDSILLVATWVTHSSWLPSGIPLQLWLPVGCGCFFLGLAL
RLVYYHWLHPSCCWKPDPVGPDFGREFARSLLSPEGYQLPQNRRMTHLAQKFFPK
AKDEAASPVKG*
YW1 granzyme B reporter synthetic cleavage site DNA sequence
(SEQ ID NO: 3)
GTGGGACCTGATTTTGGTAGAGAATTC
YW1 granzyme B reporter synthetic cleavage site amino acid sequence
(SEQ ID NO: 4)
VGPDFGREF
YW3: hXKR8 GZMB reporter with GS Linker (LGb-XKR8) reporter gene DNA
sequence (SEQ ID NO: 5)
ATGCCCTGGAGTAGTCGCGGGGCTCTCCTGCGGGACCTTGTGCTGGGAGTACTC
GGGACAGCGGCGTTCCTGTTGGACCTCGGAACTGACTTGTGGGCCGCCGTCCAG
TACGCACTTGGTGGAAGGTACCTTTGGGCGGCGCTGGTCCTGGCCCTCTTGGGG
CTGGCAAGCGTCGCTCTCCAGCTCTTTAGCTGGCTGTGGCTTCGCGCAGATCCC
GCTGGGCTGCATGGGTCCCAGCCGCCAAGGAGATGCCTGGCTCTGCTCCATCTT
CTCCAGCTCGGGTATCTTTACAGATGCGTACAAGAGTTGCGCCAGGGCCTTCTT
GTTTGGCAACAAGAGGAACCAAGTGAGTTCGACCTCGCCTATGCGGATTTCCTT
GCGTTGGATATCTCCATGCTTCGGCTCTTCGAAACATTCCTTGAGACCGCGCCA
CAATTGACCCTTGTACTTGCAATCATGCTGCAATCTGGACGAGCAGAATACTAC
CAATGGGTGGGAATCTGCACATCCTTCCTGGGCATCAGTTGGGCCCTCCTTGAT
TATCATCGCGCCTTGAGAACTTGTTTGCCAAGCAAACCATTGTTGGGCCTCGGA
TCCTCTGTTATTTATTTTCTCTGGAATCTGCTGCTTTTGTGGCCGCGAGTACTCG
CTGTTGCGCTTTTTTCCGCGTTGTTCCCTTCCTACGTCGCGCTCCATTTTCTCGGC
CTGTGGCTGGTTCTGCTGTTGTGGGTTTGGCTGCAAGGGACGGACTTTATGCCA
GACCCGTCCAGTGAGTGGCTTTACCGGGTTACAGTTGCGACCATACTTTATTTC
TCCTGGTTTAATGTCGCAGAGGGACGAACTCGCGGGAGAGCCATAATCCACTTC
GCATTCCTCCTCTCAGATTCAATACTCCTGGTCGCCACCTGGGTAACACACTCA
TCATGGCTCCCAAGTGGGATACCTTTGCAATTGTGGTTGCCGGTTGGCTGCGGG
TGTTTCTTCCTGGGTCTCGCTCTTAGACTTGTCTATTATCATTGGCTGCACCCGA
GTTGCTGCTGGAAGCCTGACCCGGGATCGGTGGGACCTGATTTTGGTAGAGAAT
TCGGCAGTGCGCGGTCCTTGCTCTCCCCAGAAGGCTACCAGTTGCCCCAAAATA
GACGCATGACTCACCTTGCCCAGAAGTTCTTTCCCAAAGCCAAGGACGAGGCA
GCTTCTCCTGTCAAGGGGTAG
YW3: hXKR8 GZMB reporter with GS Linker (LGb-XKR8) reporter gene protein
sequence (SEQ ID NO: 6)
MPWSSRGALLRDLVLGVLGTAAFLLDLGTDLWAAVQYALGGRYLWAALVLALL
GLASVALQLFSWLWLRADPAGLHGSQPPRRCLALLHLLQLGYLYRCVQELRQGLL
VWQQEEPSEFDLAYADFLALDISMLRLFETFLETAPQLTLVLAIMLQSGRAEYYQW
VGICTSFLGISWALLDYHRALRTCLPSKPLLGLGSSVIYFLWNLLLLWPRVLAVALF
SALFPSYVALHFLGLWLVLLLWVWLQGTDFMPDPSSEWLYRVTVATILYFSWFNV
AEGRTRGRAIIHFAFLLSDSILLVATWVTHSSWLPSGIPLQLWLPVGCGCFFLGLAL
RLVYYHWLHPSCCWKPDPGSVGPDFGREFGSARSLLSPEGYQLPQNRRMTHLAQK
FFPKAKDEAASPVKG*
YW3 granzyme B reporter synthetic cleavage site DNA sequence
(SEQ ID NO: 7)
GGATCGGTGGGACCTGATTTTGGTAGAGAATTCGGCAGT
YW3 granzyme B reporter synthetic cleavage site amino acid sequence
(SEQ ID NO: 8)
GSVGPDFGREFGS
*Included in any and all tables described herein are nucleic acid and polypeptide molecules having sequences with at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a respective sequence of any SEQ ID NO listed in the tables, or a portion thereof. Such polypeptides may have a function of the full-length peptide or polypeptide as described further herein.

### III. Nucleic Acids, Vectors, and Cells

In certain aspects, the present invention relates to a nucleic acid sequence encoding the reporters of phospholipid scrambling described herein. Typically, said nucleic acid is a DNA or RNA molecule, which may be included in any suitable vector, such as a plasmid, cosmid, episome, artificial chromosome, phage or a viral vector. In some embodiments, the nucleic acid comprises (e.g., consists of) a nucleotide sequence having at least 80%, 85%, 90%, 95%, 98%, or 99% identify with SEQ ID NO: 1 or 5. In some embodiments, the nucleic acid comprises (e.g., consists of) a nucleotide sequence set forth in SEQ ID NO: 1 or 5.

In some embodiments, the composition comprises an expression vector comprising an open reading frame encoding a reporter of phospholipid scrambling described herein. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the reporter of phospholipid scrambling described herein.

Examples of promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described hereinabove. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include, but are not limited to, biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

The terms “vector”, “cloning vector” and “expression vector” refer to a vehicle by which a DNA or RNA sequence (e.g., a foreign gene) may be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (KuwanaY. et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus, lentivirus, and adeno-associate virus (AAV) vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in PCT Publ. WO 95/14785, PCT Publ. WO 96/22378, U.S. Pat. Nos. 5,882,877, 6,013,516, 4,861,719, 5,278,056, and PCT Publ. WO 94/19478.

A further object encompassed by the present invention relates to a cell which has been transfected, infected or transformed by a nucleic acid and/or a vector according to the invention. The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al. 1980), rat YB2/3HL.P2.G11.16Ag.20 cell (ATCC CRL 1662, hereinafter referred to as “YB2/0 cell”), and the like. The YB2/0 cell is useful since ADCC activity of chimeric or humanized antibodies is enhanced when expressed in this cell.

The present invention also relates to a method of producing a recombinant host cell expressing a reporter of phospholipid scrambling described herein. In some embodiments, the recombinant host cell comprises the reporter of phospholipid scrambling in addition to any endogenous apoptosis-mediated scramblase possessed by the cell (e.g., in order to provide enhanced phospholipid scrambling activity as compared to the level of phospholipid scrambling activity resulting from the endogenous apoptosis-mediated scramblase). In some embodiments, the method comprises introducing in vitro or ex vivo a recombinant nucleic acid or a vector as described herein into a competent host cell and culturing in vitro or ex vivo the recombinant host cell obtained. In some embodiments, the cells which express said reporter of phospholipid scrambling may optionally be selected. Such recombinant host cells may be used for the methods encompassed by the present invention, such as the screening methods described herein.

In another aspect, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least 80% full-length sequences, at least 85% full-length sequences, at least 90% full-length sequences, at least 95% full-length sequences, or at least 99% full-length sequences, or more. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. The polynucleotides of this invention embrace nucleic acid sequences that may be employed for selective hybridization to a polynucleotide encompassed by the present invention. See, e.g., Ausubel, supra; Colligan, supra, each entirely incorporated herein by reference.

In certain aspects, provided herein are cells (e.g., antigen presenting cells) that comprise the reporters of phospholipid scrambling described herein. In certain embodiments, the cell further comprises at least one additional reporter of phospholipid scrambling. Such a reporter can be, for example, a GzB-activated infrared fluorescent protein (IFP) reporter that comprises a modified IFP comprising an internal GzB cleavage site described in the representative, non-limiting examples below. Productive antigen recognition may be identified, for example, by detection of phospholipid scrambling that results from antigen recognition rather than measuring responding cells directly. In some embodiments, the cells further comprises at least one additional reporter for cells that have the recognized antigen but is independent of serine protease or caspase cleavage, e.g., a caspase-activatable fluorescent reagent, such as CellEvent™.

In some embodiments, the cells may further be engineered, such as by transfection or genetic modification, to express exogenous nucleic acid encoding a candidate antigen. In some embodiments, such cells is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that may, in some cases, complex with peptide antigens of polypeptides, including peptide antigens processed by the cell machinery. In some cases, MHC molecules may be displayed or expressed on the cell surface, including as a complex with peptide, i.e., peptide antigen-major histocompatibility complex (pMHC) complex, for presentation of an antigen in a conformation recognizable by TCRs on T cells, or other peptide binding molecules. “MHC matching” refers to the presence of certain MHC serotypes in the context of a cognate receptor from a cytotoxic T cell and/or an NK cell that recognizes the MHC serotype in the context of a pMHC complex. In some embodiments, cytotoxic lymphocytes are engineered to express a TCR or other receptor that recognizes pMHC complexes, such as a library of recombinant cytotoxic lymphocytes expressing a diversity of such receptors, which can be constructed according to library generation methods described herein. In some embodiments, the endogenous TCR or other receptor that recognizes pMHC complexes are deleted, mutated, silenced, or otherwise prevented from being expressed.

In some embodiments, the cell is a primary cell or a cell of a cell line. In some embodiments, the cell is a nucleated cell. In some embodiments, the cell is an antigen-presenting cell. In some embodiments, the cell is a macrophage, dendritic cell, B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s), Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15, IL-21, interferon-alpha (IFNα), interferon-beta (IFNβ), interferon-gamma (IFNγ), tumor necrosis factor-alpha (TNFα), tumor necrosis factor-beta (TNFβ), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, U.S. Pat. Publ. US 2014/0212446; Butler and Hirano (2014) Immunol Rev. 257:10.1111/imr.12129; Suhoshki et al. (2007) Mol. Ther. 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well known in the art. In some embodiments, the MHC typing of cells, such as primary cells obtained from a subject, may be determined using procedures well known in the art, such as by performing tissue typing using molecular haplotype assays (BioTest ABC SSPtray, BioTest Diagnostics Corp., Denville, N.J.; SeCore Kits, Life Technologies, Grand Island, N.Y.). In some cases, it is well within the level of a skilled artisan to perform standard typing of cells to determine the HLA genotype, such as by using sequence-based typing (SBT) (Adams et al. (2004) J. Transl. Med. 2:30; Smith (2012) Methods Mol. Biol. 882:67-86). In some cases, the HLA typing of cells, such as fibroblast cells, are known. For example, the human fetal lung fibroblast cell line MRC-5 is HLA-A*0201, A29, B13, B44 Cw7 (C*0702); the human foreskin fibroblast cell line Hs68 is HLA-A1, A29, B8, B44, Cw7, Cw16; and the WI-38 cell line is A*6801, B*0801, (Solache et al. (1999) J. Immunol. 163:5512-5518; Ameres et al. (2013) PloS Pathog. 9:e1003383). The human transfectant fibroblast cell line M1DR1/Ii/DM express HLA-DR and HLA-DM (Karakikes et al. (2012) FASEB J. 26:4886-4896).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

Serine proteases like granzyme B initiates caspase activation in target cells, which leads to internucleosomal degradation of genomic DNA by the caspase-activated deoxyribonuclease (CAD). Accordingly, in order to recover nucleic acids that encode recognized antigens, DNA degradation (e.g., caspase-activated deoxyribonuclease (CAD)-mediated DNA degradation) may be blocked in the cells. For example, in some embodiments, the cells may further comprise an inhibitor of DNA degradation, such as inhibitors of the CAD-mediated DNA degradation. Methods of reducing or blocking degradation of genomic DNA are known in the art. For example, the cells may be modified to express the inhibitor of caspase-activated DNase (ICAD) protein to inhibit degradation of genomic DNA. In certain embodiments, the cell is modified to overexpress ICAD, or to express an ICAD mutant with increased activity. In some embodiments, the ICAD contains a mutation conferring resistance to caspase cleavage (e.g., D117E and/or D224E), otherwise referred to herein as a caspase resistant mutant (Sakahira et al. (2001) Arch. Biochem. Biophys. 388:91-99; Enari et al. (1998) Nature 391:43-50; Sakahira et al. (1998) Nature 391:96-99).

Compositions and methods for inhibiting CAD-mediated DNA degradation are well-known in the art (see, for example, U.S. Pat. Publ. 2020/0102553 and Kula et al. (2019) Cell 178:1016-1028). For example, in some embodiments, the copy number, level and/or activity of CAD may be reduced in the cells. For example, the CAD gene may be disrupted in the cells (e.g., using CRISPR, TALEN, or other genome-editing tools), or knockdown (e.g., using an inhibitory nucleic acid such as shRNA, siRNA, LNA, or antisense). Multiple siRNA, shRNA, CRISPR constructs for reducing CAD expression are commercially available, such as shRNA product #TL314229, siRNA product SR300555, and CRISPR products #GA100553 and GA208294 from Origene Technologies (Rockville, Md.). Chemical or small molecule DNAse inhibitors may also be used, e.g., Mirin, a cell-permeable inhibitor of the Mrel 1 nuclease, or intercalating dyes like ethidium bromide, that inhibit proteins that interact with nucleic acids.

Caspase 3 initiates DNA degradation by cleaving DFF45 (DNA fragmentation factor-45)/ICAD (inhibitor of caspase-activated DNase) to release the active enzyme CAD (Wolf et al. (1999) J. Biol. Chem. 274:30651-30656). Thus, caspase inhibition may also be used to prevent cleavage of ICAD and resulting activation of CAD during apoptosis. In some embodiments, the cells may include a caspase 3 knockout TALEN, or other genome-editing tools), or knockdown (e.g., using an inhibitory nucleic acid such as shRNA, siRNA, LNA, or antisense). Multiple siRNA, shRNA, CRISPR constructs for reducing caspase 3 expression are commercially available, such as shRNA product #TL305638, siRNA product SR300591, and CRISPR products #GA100589 and GA200538 from Origene Technologies (Rockville, Md.). Chemical or small molecule caspase inhibitors may also be used, which include but are not limited to, e.g., Z-VAD-FMK (Benzyl oxycarbonyl-Val-Ala-Asp(OMe)-fluoromethylketone), Z-DEVD-FMK, Ac-DEVD-CHO; Q-VD-Oph (Quinolyl-Val-Asp-OPh), M826 (Han et al. (2002) J. Biol. Chem. 277:30128-30136), N-benzylisatin sulfonamide analogues as described in Chu et al. (2005) J. Med. Chem. 48:7637-7647, and isoquinoline-1,3,4-trione derivatives as described in Chen et al. (2006) J. Med. Chem. 49:1613-1623). Protein or peptide inhibitors of caspases may also be used, which include but are not limited to, e.g., mammalian X-linked inhibitor of apoptosis (XIAP) or cowpox CrmA. Because ICAD may be cleaved and activated by other caspases, inhibitors of other caspases may also be used, e.g., pan-caspase inhibitors, or inhibitors of executioner caspases (caspase 6 or 7) or initiator caspases (caspase 2, 8, 9, or 10). In some embodiments, the caspase inhibitor inhibits both caspase 3 and other caspases, such as caspase 6, 7, 2, 8, and/or 9.

### IV. Libraries of Target Cells

Also provided herein are libraries of target cells comprising reporters of phospholipid scrambling described herein and a plurality of candidate antigens. In some embodiments, the library of target cells may comprise a plurality of cells (e.g., antigen presenting cells) modified as described herein, wherein the cells (e.g., antigen presenting cells) comprise reporters of phospholipid scrambling described herein, and different exogenous nucleic acids (e.g., DNA or RNA) encoding candidate antigens, such that plurality of cells (e.g., antigen presenting cells) collectively present a library of candidate antigens. In some embodiments, each cell contains and expresses a single nucleic acid, perhaps in multiple copies, to thereby present a single candidate antigen with MHC class I and/or MHC class II molecule. In other embodiments, each cell (e.g., antigen presenting cell) contains and expresses a handful of different nucleic acids expressing different candidate antigens, perhaps in multiple copies, to thereby present several candidate antigens (e.g., 2, 3, 4, 5, 6, or more) with MHC class I and/or MHC class II molecules.

In some embodiments, the library of target cells may comprise a plurality of cells (e.g., antigen presenting cells) modified as described herein, wherein the cells (e.g., antigen presenting cells) comprise reporters of phospholipid scrambling described herein, and different candidate antigens bound to MHC class I and/or MHC class II molecule, such that the plurality of cells (e.g., antigen presenting cells) collectively present a library of candidate antigens. In some embodiments, the library of candidate antigens are mixed with the target cells comprising reporters of phospholipid scrambling described herein under appropriate conditions such that the candidate antigens are loaded to MHC class I and/or MHC class II molecules of the target cells. In other embodiments, polypeptides, cells or organisms are internalized and processed by the target cells comprising reporters of phospholipid scrambling described herein, and presented by the target cells with MHC class I and/or MHC class II molecules.

The exogenous nucleic acids (e.g., DNA or RNA) encoding candidate antigens may be introduced into target cells by transfection and/or transduction using conventional techniques. In some embodiments, target cells are transduced using a viral vector, such as a lentivirus, which results in a stable viral integration into the target cell genome. Transduction is carried out under conditions that result in on average no more than one viral integration event per target cell. Transduction techniques include, but are not limited to, lipofection, electroporation, and the like. Methods for the construction of large, genome-scale libraries of sequences for the expression of encoded polypeptides, such as in the generation of the candidate antigen libraries to be introduced into MHC target cells, are known in the art. Exemplary methods are described in Xu et al. (2015) Science 348:aaa0698; Larman et al. (2011) Nat. Biotechnol. 29:535-41; Zhu et al. (2013) Nat. Biotechnol. 31:331-334).

In some embodiments, a library of antigen-expressing vectors is transfected into aAPCs. An antigen coding sequence may be for the peptide of interest, a minigene construct or an entire cDNA coding sequence which may be processed appropriately into peptides prior to MHC class I and/or MHC class II binding and surface display. Peptides may also be directly added to the aAPCs for MHC loading. The antigen library may be composed of an unbiased set of protein coding regions from the target cell of interest or may be more narrowly defined (e.g., neoantigens determined by exome sequencing, virus-derived genes).

In some embodiments, caspase-activated deoxyribonuclease (CAD)-mediated DNA degradation is blocked in the target cells. Numerous representative examples of agents that may reduce or inhibit CAD-mediated DNA degradation are described herein. For example, the target cells may comprise an exogenous inhibitor of CAD-mediated DNA degradation, or a CAD or caspase (e.g., caspase 3) knockout or knockdown, such as those described herein. For example, in some embodiments, the exogenous inhibitor of CAD-mediated DNA degradation is a nucleic acid encoding inhibitor of caspase-activated deoxyribonuclease (ICAD) gene in expressible form, an inhibitory nucleic acid targeting CAD or caspase 3, a small molecule inhibitor of caspase 3, a chemical DNAse inhibitor, or a peptide or protein inhibitor of caspase 3. The ICAD gene may be wild type or a caspase-resistant ICAD mutant. The caspase-resistant ICAD mutant may comprise mutation D117E (i.e., the aspartic acid at position 117 is substituted with a glumatic acid), and/or D224E (i.e., the aspartic acid at position 224 is substituted with a glumatic acid).

In some embodiments, the target cells further comprise one or more additional reporters useful in identification of an activated target cell, such as those described herein. In some embodiments, the additional reporter is sensitive to granzyme B activity, such as GzB-activatable IFP reporter. In some embodiments, the additional reporter is independent of granzyme B cleavage, e.g., a caspase-activatable fluorescent reagent, such as CellEvent™ or caspase-3/7 detection reagents.

In some embodiments, the size of the library of candidate antigens varies from about 100 members to about 1×10¹⁴ members; about 1×10³ to about 10¹⁴ members, about 1×10⁴ to about 10¹⁴ members, about 1×10⁵ to about 10¹⁴ members, about 1×10⁶ to about 10¹⁴ members, about 1×10⁷ to about 10¹⁴ members, about 1×10⁸ to about 10¹⁴ members, about 1×10⁹ to about 10¹⁴ members, about 1×10¹⁰ to about 10¹⁴ members, about 1×10¹¹ to about 10¹⁴ members, about 1×10¹² to about 10¹⁴ members, about 1×10¹³ to about 10¹⁴ members, or about 1×10¹⁴ members. In some embodiments, the library of candidate antigens comprises at least 100 member sequences, for example, at least 10³ members, at least 10⁴ members, at least 10⁵ members, at least 10⁶ members, at least 10⁷ members, at least 10⁸ members, at least 10⁹ members, at least 10¹⁰ members, at least 10¹¹ members, at least 10¹² members, at least 10¹³ members. In some embodiments, epitope-encoding libraries comprise up to 10¹⁴ member sequences, for example, up to 10¹³ members, up to 10¹² members, up to 10¹¹ members, up to 10¹⁰ members, up to 10⁹ members, up to 10⁸ members, up to 10⁷ members, up to 10⁶ members, up to 10⁵ members, up to 10⁴ members, up to 10³ members, and the like.

In some embodiments, each target cell encodes a unique candidate antigen. In other embodiments, a target cell may encode more than one unique candidate antigen, such as 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more, or any range in between, inclusive (e.g., 5-10) candidate antigens per cell. If the screen results in higher background when using multiple antigens per cell, the methods may include performing one or more additional rounds of the screen with just one antigen per cell (in some embodiments, re-cloned antigens from the first or an earlier pass).

The library of cells (e.g., antigen presenting cells) may be derived from the same cell type. For example, e.g., they were clonal prior to modification. In some embodiments, the library is made of a plurality of cells (e.g., antigen presenting cells) that are an isolated population and/or are substantially pure population of cells. Examples of suitable cells include but are not limit to a K562 cell, a HEK 293 cell, a HEK 293 T cell, a U2OS cell, MelJuso cell, a MDA-MB231 cell, a MCF7 cell, a NTERA2a cell, a dendritic cell, a macrophage and a primary autologous B cell.

In some embodiments, the library of target cells may comprise about 1×10² to about 10¹⁴ target cells, about 1×10³ to about 10¹⁴ target cells, about 1×10⁴ to about 10¹⁴ target cells, about 1×10⁵ to about 10¹⁴ target cells, about 1×10⁶ to about 10¹⁴ target cells, about 1×10⁷ to about 10¹⁴ target cells, about 1×10⁸ to about 10¹⁴ target cells, about 1×10⁹ to about 10¹⁴ target cells, about 1×10¹⁰ to about 10¹⁴ target cells, about 1×10¹¹ to about 10¹⁴ target cells, about 1×10¹² to about 10¹⁴ target cells, about 1×10¹³ to about 10¹⁴ target cells, or about 1×10¹⁴ target cells. The target cell libraries described herein provide at least about 10² to about 10¹⁴ candidate antigens, wherein a sufficient amount of target cells comprise a unique candidate antigen for effective library screening. In some embodiments, a representation of between 10 and 10,000 is used, meaning each candidate antigen is presented by 10-10,000 cells.

The antigen may be encoded at single copy at the DNA level. From the single copy of the DNA, tens to thousands of antigen molecules may be produced, processed and presented with MHC per cell. Even single peptides on the surface of the cell, however, can be productively recognized by cytotoxic lymphocyte, such as a cytotoxic T cell and/or an NK cell, and so the system is functional for even very low copies of surface expressed antigen.

In some embodiments, each target cell comprises about 10² to about 10¹⁴ molecules of the candidate antigen. In exemplary embodiments, each target cell comprises about 1×10² to about 10¹⁴ copies of the candidate antigen, about 1×10³ to about 10¹⁴ copies of the candidate antigen, about 1×10⁴ to about 10¹⁴ copies of the candidate antigen, about 1×10⁵ to about 10¹⁴ copies of the candidate antigen, about 1×10⁶ to about 10¹⁴ copies of the candidate antigen, about 1×10⁷ to about 10¹⁴ copies of the candidate antigen, about 1×10⁸ to about 10¹⁴ copies of the candidate antigen, about 1×10⁹ to about 10¹⁴ copies of the candidate antigen, about 1×10¹⁰ to about 10¹⁴ copies of the candidate antigen, about 1×10¹¹ to about 10¹⁴ copies of the candidate antigen, about 1×10¹² to about 10¹⁴ copies of the candidate antigen, about 1×10¹³ to about 10¹⁴ copies of the candidate antigen, or about 1×10¹⁴ copies of the candidate antigen.

A wide variety of libraries of epitope-encoding nucleic acids may be used, which differ in size and structure of member sequences. Generally libraries encode peptides that are capable of being processed by the MHC presentation and transport mechanisms of the target cells. In some embodiments, libraries comprise nucleic acids capable of encoding peptides at least 8 amino acids in length; in other embodiments, libraries comprise nucleic acids capable of encoding peptides at least 10 amino acids in length; in other embodiments, libraries comprise nucleic acids capable of encoding peptides at least 14 amino acids in length; in other embodiments, libraries comprise nucleic acids capable of encoding peptides at least 20 amino acids in length. In some embodiments, the candidate antigens are encoded by nucleic acids that are about 21 to about 150 nucleotides in length, about 24 to about 150 nucleotides in length, about 30 to about 150 nucleotides in length, about 40 to about 150 nucleotides in length, about 50 to about 150 nucleotides in length, about 60 to about 150 nucleotides in length, about 70 to about 150 nucleotides in length, about 80 to about 150 nucleotides in length, about 90 to about 150 nucleotides in length, about 100 to about 150 nucleotides in length, about 110 to about 150 nucleotides in length, about 120 to about 150 nucleotides in length, about 130 to about 150 nucleotides in length, about 140 to about 150 nucleotides in length or about 150 nucleotides in length. In some embodiments, the ORF or nucleic acid encoding the candidate antigen is longer than 150 nt. In some embodiments, the epitopes are, or are processed upon expression to become, 8, 9, 10, 11, 12, 13, 14, and/or 15 amino acids in length.

In some embodiments, the candidate antigens are at least 20, at least 30, at least 40, at least 50, at least 60, at least 70, at least 80, at least 90, at least 100, at least 150, at least 200, at least 250, at least 300, at least 350, at least 400, at least 450 amino acids or more in length. For example, an candidate antigen or epitope may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein.

Upon expression, longer antigens (e.g., hundreds of amino acids) may be processed down into short peptides that are displayed on the surface of the target cells. In some embodiments, the candidate antigens displayed on the surface of target cells are 8-24 amino acids long. In some embodiments, an antigen or epitope thereof for MHC class I is 13 residues or less in length, for example, between about 8 and about 11 residues, and, in some embodiments, 9 or 10 residues. In some embodiments, an immunogenic antigen or epitope thereof for MHC class II is 9-24 residues in length. Identification of a target cell having a nucleic acid encoding a long candidate antigen may be followed by further screening of various fragments of the identified candidate.

In some embodiments, the candidate antigens bind to the lymphocyte with a Kd of from about 1 fM to about 100 μM, about 1 pM to about 100 μM, about 100 nM to about 100 μM, about 1 μM to about 100 μM, about 1 μM to about 10 μM, about 1 pM to about 100 nM, about 1 pM to about 10 nM, about 1 pM to about 5 nM. In some embodiments, the candidate antigens bind to the lymphocyte with a Kd of 1 mM.

Techniques for constructing libraries encoding peptides and polypeptides are well-known in the art, such as where libraries are provided that comprise sequences of codons of various compositions. In some embodiments, where an epitope-encoding library is derived from a protein, members of such library may comprise nucleic acids encoding overlapping peptide segments of the protein. The lengths and degree of overlap of such peptides is a design choice for implementing the invention. In some embodiments, an epitope-encoding library includes a nucleic acids encoding every peptide segment of a collection of segments that covers the pre-determined protein. In a further embodiment, such collection includes a series of segments of the same length each shifted by one amino acid along the length of the protein.

In some embodiments, epitope-encoding libraries for use with the invention may comprise random nucleotide sequences of a pre-determined length, e.g., at least 24 nucleotides or greater in length. In other embodiments, epitope-encoding libraries for use with the invention may comprise sequences of randomly selected codons of a pre-determined length, e.g., comprising a length of at least eight codons or more. In other embodiments, epitope-encoding libraries for use with the invention may comprise sequences of randomly selected codons of a pre-determined length, e.g., comprising a length of at least 14 codons or more. In other embodiments, epitope-encoding libraries for use with the invention may comprise sequences of randomly selected codons of a pre-determined length, e.g., comprising a length of at least 20 codons or more.

In other embodiments, epitope-encoding libraries depend on the tissue, lesion, sample, exome or genome of an individual from whom T cell epitopes are being identified. Epitope-encoding libraries may be derived from genomic DNA (gDNA), exomic DNA or cDNA. More particularly, epitope-encoding libraries may be derived from gDNA or cDNA from tumor tissue, microbially infected tissue, autoimmune lesions, graft tissue pre or post-transplant (to identify alloantigens), or gDNA from a microbiome sample, gDNA from a microbial (i.e., viral, bacterial, fungal, etc.) isolate. That is, peptides encoded by an epitope-encoding library may be derived from or represent actual coding sequences of the foregoing sources. Such libraries may comprise nucleic acids that cover, or include representatives, of all sequences in the foregoing sources or subsets of coding sequences in the foregoing sources. Such libraries based on actual coding sequences (i.e., sequences of codons) may be constructed as taught by Larman et al. (2011) Nat. Biotech. 29:535-541. Briefly, such methods comprising the steps of massively parallel synthesis on a microarray of epitope-encoding regions sandwiched between primer binding sites; cleaving or releasing synthesized sequences from the microarray; optionally amplifying the sequences; and cloning such sequences into a vector carrying the library. One of ordinary skill in the art would understand that such nucleic acid sequences would be inserted into an expression vector in an “in-frame” configuration with respect to promoter (and/or other) vector elements so that the amino acid sequences of peptides expressed correspond to those of the peptides found in the foregoing sources.

In some embodiments, epitope-encoding libraries are prepared from cDNA or gDNA from an individual whose T cell epitopes are being identified. In particular, when such individual is a cancer patient, such cDNA, gDNA, exome sequences, or the like, may be obtained, or extracted from, a cancerous tissue of the individual. In some embodiments, epitope-encoding libraries may be derived from sequences of cDNAs determined by cancer antigen-discovery techniques, such as, for example, SEREX (disclosed in Pfreundschuh, U.S. Pat. No. 5,698,396, which is incorporate herein by reference), and like techniques.

In still other embodiments, selection of epitope-encoding nucleic acids for a library may be guided by in silico T cell epitope prediction methods, including, but not limited to, those disclosed in U.S. Pat. No. 7,430,476; PCT Publ. No. WO 2004/063963; Parker et al. (2010) BMC Bioinformatics 11:180; Desai et al. (2014) Methods Mol. Biol. 1184:333-364; Bhasin et al. (2004) Vaccine 22:195-204; Nielsen et al. (2003) Protein Science 12:1007-1017; Patronov et al. (2013) Open Biol. 3:120139; Lundegaard et al. (2012) Expert Rev. Vaccines 11:43-54; and the like. Briefly, candidate epitope-encoding nucleic acid sequences may be selected from all or parts (e.g., overlapping segments) of nucleic acids, e.g., genes or exons, encoding one or more proteins of an individual. In some embodiments, such protein-encoding nucleic acids may be obtained by sequencing all or part of an individual's genome. In other embodiments, such protein-encoding nucleic acids may be obtained from known cancer genes, including their common mutant forms.

In some embodiments, the library of candidate antigens may be designed to include full-length polypeptides and/or portions of polypeptides encoded by an infectious agent or target cell. Expression of full length polypeptides maximizes epitopes available for presentation by a human antigen presenting cell, thereby increasing the likelihood of identifying an antigen. However, in some embodiments, it is useful to express portions of ORFs, or ORFs that are otherwise altered, to achieve efficient expression. For example, in some embodiments, ORFs encoding polypeptides that are large (e.g., greater than 1,000 amino acids), that have extended hydrophobic regions, signal peptides, transmembrane domains, or domains that cause cellular toxicity, are modified (e.g., by C-terminal truncation, N-terminal truncation, or internal deletion) to reduce cytotoxicity and permit efficient expression a library cell, which in turn facilitates presentation of the encoded polypeptides on human cells. Other types of modifications, such as point mutations or codon optimization, may also be used to enhance expression.

The number of polypeptides included in a library may be varied. A library may be designed to express polypeptides from at least 5%, 10%, 15%, 20%, 25%, 35%, 40%, 45%, 50%, 55%, 60%, 70%, 75%, 80%, 85%, 90%, 95%, 97%, 98%, 99%, or more, of the ORFs in an infectious agent or target cell. In some embodiments, a library expresses at least 25, 50, 75, 100, 150, 200, 250, 300, 350, 400, 450, 500, 550, 600, 650, 700, 750, 800, 850, 900, 950, or 1000 different heterologous polypeptides, each of which may represent a polypeptide encoded by a single full length ORF or portion thereof.

In some embodiments, it is advantageous to include polypeptides from as many ORFs as possible, to maximize the number of candidate antigens for screening. In some embodiments, a subset of polypeptides having a particular feature of interest is expressed. For example, for assays focused on identifying antigens associated with a particular stage of infection, an ordinarily skilled artisan may construct a library that expresses a subset of polypeptides associated with that stage of infection (e.g., a library that expresses polypeptides associated with the hepatocyte phase of infection by Plasmodium falciparum, e.g., a library that expresses polypeptides associated with a yeast or mold stage of a dimorphic fungal pathogen). In some embodiments, assays may focus on identifying antigens that are secreted polypeptides, cell surface-expressed polypeptides, or virulence determinants, e.g., to identify antigens that are likely to be targets of both humoral and cell mediated immune responses.

In some embodiments, the exogenous nucleic acid encoding a candidate antigen is derived from a virus. For example, the library of target cells may be designed to express candidate antigens from one of the following viruses: an immunodeficiency virus (e.g., a human immunodeficiency virus (HIV), e.g., HIV-1, HIV-2), a hepatitis virus (e.g., hepatitis B virus (HBV), hepatitis C virus (HCV), hepatitis A virus, non-A and non-B hepatitis virus), a herpes virus (e.g., herpes simplex virus type I (HSV-1), HSV-2, Varicella-zoster virus, Epstein Barr virus, human cytomegalovirus, human herpesvirus 6 (HHV-6), HHV-8), a poxvirus (e.g., variola, vaccinia, monkeypox, Molluscum contagiosum virus), an influenza virus, a human papilloma virus, adenovirus, rhinovirus, coronavirus, respiratory syncytial virus, rabies virus, coxsackie virus, human T-cell leukemia virus (types I, II and III), parainfluenza virus, paramyxovirus, poliovirus, rotavirus, rhinovirus, rubella virus, measles virus, mumps virus, adenovirus, yellow fever virus, Norwalk virus, West Nile virus, a Dengue virus, Severe Acute Respiratory Syndrome Coronavirus (SARS-CoV), bunyavirus, Ebola virus, Marburg virus, Eastern equine encephalitis virus, Venezuelan equine encephalitis virus, Japanese encephalitis virus, St. Louis encephalitis virus, Junin virus, Lassa virus, and Lymphocytic choriomeningitis virus. Libraries for other viruses may also be produced and used according to methods described herein.

In some embodiments, the exogenous nucleic acid encoding a candidate antigen is derived from bacteria (e.g., from a bacterial pathogen). In some embodiments, the bacterial pathogen is an intracellular pathogen. In some embodiments, the bacterial pathogen is an extracellular pathogen. Examples of bacterial pathogens include bacteria from the following genera and species: Chlamydia (e.g., Chlamydia pneumoniae, Chlamydia psittaci, Chlamydia trachomatis), Legionella (e.g., Legionella pneumophila), Listeria (e.g., Listeria monocytogenes), Rickettsia (e.g., R. australis, R. rickettsia, R. akari, R. conorii, R. sibirica, R. japonica, R. africae, R. typhi, R. prowazekii), Actinobacter (e.g., Actinobacter baumannii), Bordetella(e.g., Bordetella pertussis), Bacillus (e.g., Bacillus anthracis, Bacillus cereus), Bacteroides (e.g., Bacteroides fragilis), Bartonella (e.g., Bartonella henselae), Borrelia (e.g., Borrelia burgdorferi), Brucella (e.g., Brucella abortus, Brucella canis, Brucella melitensis, Brucella suis), Campylobacter (e.g., Campylobacter jejuni), Clostridium (e.g., Clostridium botulinum, Clostridium difficile, Clostridium perfringens, Clostridium tetani), Corynebacterium (e.g., Corynebacterium diphtheriae, Corynebacterium amycolatum), Enterococcus (e.g., Enterococcus faecalis, Enterococcus faecium), Escherichia (e.g., Escherichia cob), Francisella (e.g., Francisella tularensis), Haemophilus (e.g., Haemophilus influenzae), Helicobacter (e.g., Helicobacter pylori), Klebsiella (e.g., Klebsiella pneumoniae), Leptospira (e.g., Leptospira interrogans), Mycobacteria (e.g., Mycobacterium leprae, Mycobacterium tuberculosis), Mycoplasma (e.g., Mycoplasma pneumoniae), Neisseria (e.g., Neisseria gonorrhoeae, Neisseria meningitidis), Pseudomonas (e.g., Pseudomonas aeruginosa), Salmonella (e.g., Salmonella typhi, Salmonella typhimurium, Salmonella enterica), Shigella (e.g., Shigella dysenteriae, Shigella sonnei), Staphylococcus (e.g., Staphylococcus aureus, Staphylococcus epidermidis, Staphylococcus saprophyticus), Streptococcus (e.g., Streptococcus agalactiae, Streptococcus pneumoniae, Streptococcus pyogenes), Treponoma (e.g., Treponoma pallidum), Vibrio (e.g., Vibrio cholerae, Vibrio vulnificus), and Yersinia (e.g., Yersinia pestis). Libraries for other bacteria may also be produced and used according to methods described herein.

In some embodiments, the exogenous nucleic acid encoding a candidate antigen is derived from protozoa. Examples of protozoal pathogens include the following organisms: Cryptosporidium parvum, Entamoeba (e.g., Entamoeba histolytica), Giardia (e.g., Giardia lambila), Leishmania (e.g., Leishmania donovani), Plasmodium spp. (e.g., Plasmodium falciparum, Plasmodium vivax, Plasmodium ovale, Plasmodium malariae), Toxoplasma (e.g., Toxoplasma gondii), Trichomonas (e.g., Trichomonas vaginalis), and Trypanosoma (e.g., Trypanosoma brucei, Trypanosoma cruzi). Libraries for other protozoa may also be produced and used according to methods described herein.

In some embodiments, the exogenous nucleic acid encoding a candidate antigen is derived from a fungus. Examples of fungal pathogens include the following: Aspergillus, Candida (e.g., Candida albicans), Coccidiodes (e.g., Coccidiodes immitis), Cryptococcus (e.g., Cryptococcus neoformans), Histoplasma (e.g., Histoplasma capsulatum), and Pneumocystis (e.g., Pneumocystis carinii). Libraries for other fungi may also be produced and used according to methods described herein.

In some embodiments, the exogenous nucleic acid encoding a candidate antigen is derived from helminth. Examples of helminthic pathogens include Ascaris lumbricoides, Ancylostomna, Clonorchis sinensis, Dracuncula mnedinensis, Enterobius vermicularis, Filaria, Onchocerca volvulus, Loa loa, Schistosoma, Strongyloides, Trichuris trichura, and Trichinella spiralis. Libraries for other helminths may also be produced and used according to methods described herein.

Sequence information for genomes and ORFs for infectious agents is publicly available. See, e.g., the Entrez Genome Database (available on the World Wide Web at ncbi.nlm.nih.gov/sites/entrez?db-Genome&itool=toolbar), the ERGO™ Database (available on the World Wide Web igwcb.integratcdgcnomics.com/ERGO_supplement/genomes.html), and the Genomes Online Database (GOLD) (available on the World Wide Web at genomesonline.org) (Liolios et al. (2006) Nucl. Acids Res. 1:D332-D334).

In some embodiments, the exogenous nucleic acid encoding a candidate antigen is derived from a human DNA (e.g., a human cancer cell). Such libraries are useful, e.g., for identifying candidate tumor antigens, or targets of autoreactive immune responses. An exemplary library for identifying tumor antigens includes polynucleotides encoding polypeptides that are differentially expressed or otherwise altered in tumor cells. An exemplary library for evaluating autoreactive immune responses includes polynucleotides expressed in the tissue against which the autoreactive response is directed (e.g., a library containing pancreatic polynucleotide sequences is used for evaluating an autoreactive immune response against the pancreas).

### V. Systems for Detection of Recognized Antigen Presentation

In some aspects, provided herein are systems for detection of recognized antigen presentation by an antigen presenting cell to a cytotoxic lymphocyte (e.g., a cytotoxic T cell and/or NK cell). In some embodiments, the systems comprise an antigen presenting cell, or a plurality of antigen presenting cells, comprising (i) a reporter of phospholipid scrambling as described herein and (ii) an exogenous nucleic acid encoding a candidate antigen, wherein the candidate antigen is expressed and presented with MHC class I and/or MHC class II molecules to cytotoxic lymphocyte (e.g., a cytotoxic T cell and/or NK cell), as described herein. In some embodiments, the antigen presenting cells of the systems further comprise an inhibitor of CAD-mediated DNA degradation, such as an ICAD gene in expressible form. In some embodiments, the systems further comprise a cytotoxic lymphocyte (e.g., a cytotoxic T cell and/or NK cell).

Cytotoxic T cells and/or NK cells may be obtained from virtually any source containing such cells, including, but not limited to, peripheral blood (e.g., as a peripheral blood mononuclear cell (PBMC) preparation), dissociated organs or tissue, including tumors, synovial fluid (e.g., from arthritic joints), ascites fluid or pleural effusion form cancer patients, cerebral spinal fluid, and the like. Sources of particular interest include tissues affected by diseases, such as cancers, autoimmune diseases, viral infections, and the like. In some embodiments, cytotoxic T cells and/or NK cells used in methods encompassed by the present invention are provided as a clonal population or a near clonal population. Such populations may be produced using conventional techniques, for example, sorting by FACS into individual wells of a microtitre plate, cloning by limited dilution, and the like, followed by growth and replication. In vitro expansion of the desired cytotoxic T cells and/or NK cells may be carried out in accordance with known techniques (including but not limited to those described in U.S. Pat. No. 6,040,177), or variations thereof that are apparent to those skilled in the art.

In some embodiments, cytotoxic T cells and/or NK cells from tissues affected by cancer, such as tissue-infiltrating T lymphocytes (TILs), may be used, and may be obtained as described in Dudley et al. (2003) J. Immunotherapy 26:332-342 and Dudley et al. (2007) Semin. Oncol. 34:524-531.

In some embodiments, cytotoxic T cells and/or NK cells are modified to express an antigen receptor of interest. In some embodiments, the cytotoxic T cell and/or NK cell are modified to express a T cell receptor from a non-cytotoxic CD4 T cell. In some embodiments, the cytotoxic T cell is a cytotoxic CD4+ T cell or a cytotoxic CD8+ T cell. CD4+ T cells can assist other white blood cells in immunologic processes, including maturation of B-cells and activation of cytotoxic T cells and macrophages. CD4+ T cells are activated when presented with peptide antigens by MHC class II molecules expressed on the surface of antigen presenting cells (APCs). Once activated, the T cells can divide rapidly and secrete cytokines that regulate the active immune response. CD8+ T cells can destroy virally infected cells and tumor cells, and can also be implicated in transplant rejection. CD8+ T cells can recognize their targets by binding to antigen associated with MHC class I, which is present on the surface of nearly every cell of the body.

T cell purification may be achieved, for example, by positive or negative selection including, but not limited to, the use of antibodies directed to CD2, CD3, CD4, CD5, CD 8, CD 14, CD 19, and/or MHC class II molecules. A specific T cell subset, such as CD28⁺, CD4⁺, CD8⁺, CD45RA, and/or CD45RO T cells, may be isolated by positive or negative selection techniques. For example, CD3⁺, CD28⁺ T cells may be positively selected using CD3/CD28 conjugated magnetic beads. In one aspect encompassed by the present invention, enrichment of a T cell population by negative selection may be accomplished with a combination of antibodies directed to surface markers unique to the negatively selected cells.

As described herein, productive antigen recognition presented on the recognized target APC by the cytotoxic lymphocyte (e.g., a cytotoxic T cell and/or NK cell) results in recognizable changes within the APC. Detection of such changes may be used to identify the APC and eventual determination of the antigen(s) it expresses. In some embodiments, Identification of the recognized target cell and identification of the antigen therein, may be accomplished by use of high-throughput systems that detect the reporters within the target cells.

Isolating and/or sorting as described herein may be conducted using a variety of methods and/or devices known in the art, e.g., flow cytometry (e.g., fluorescence activated cell sorting (FACS) or Ramen flow cytometry), fluorescence microscopy, optical tweezers, micro-pipettes, affinity purification, and microfluidic magnetic separation devices and methods.

In some embodiments, when target cells comprising the candidate antigens specifically bind their cognate T cells, the reporter of the target cell is activated and promotes the translation and exposure of PS, which enables direct detection of activated scramblase (such as affinity detection of cleaved scramblase or fluorescence detection of cleaved scramblase, wherein either one or both of the activated scramblase or the cleaved portion of the scramble are tagged) or indirect detection of activated scrambles like outer leaf PS detection, such as isolation or enrichment using a physical substrate that binds to PS (e.g., by a Annexin-V bead/column).

In some embodiments, the antigen presenting cells of the systems further comprise at least one additional reporter of cytotoxic T cell and/or NK cell recognition of the peptide antigen-major histocompatibility complex (pMHC) complex presented by the antigen presenting cells, such as an alternative serine protease- or caspase-activated reporter or a reporter that is independent of serine protease or caspase activity.

In some embodiments, where the target cell comprises an additional reporter that optically labels the target cell, such as using a colored dye, fluorescent label, and the like (e.g., the GzB-activated IFP reporter), FACS may be utilized to quantitatively sort the cells based on one or more fluorescence signals. FACS may be used to sort the bound cells from the unbound cells based on the infrared fluorescent signal. One or more sort gates or threshold levels may be utilized in connection with one or more detection molecules to provide quantitative sorting over a wide range of target cell-T cell interactions. In addition, the screening stringency may be quantitatively controlled, e.g., by modulating the target concentration and setting the position of the sort gates.

Where, for example, the fluorescence signal is related to the binding affinity of the candidate antigen to the cytotoxic lymphocyte (e.g., a cytotoxic T cell and/or NK cell), the sort gates and/or stringency conditions may be adjusted to select for antigens having a desired affinity or desired affinity range for the target. In some cases, it may be desirable to isolate the highest affinity antigens from a particular library of candidate antigens sequences. However, in other cases candidate antigens falling within a particular range of binding affinities may be isolated.

Cells identified as having recognized antigen may be processed to isolate the exogenous nucleic acid. A variety of conventional techniques may be used to analyze epitope-encoding nucleic acids from target cells that have been induced to generate a signal indicating recognition and activation of a cognate T cell. In some embodiments, such target cells are first isolated then, in turn, the epitope-encoding nucleic acids are isolated from such cells. For example, in some embodiments epitopes are expressed from plasmids so that the encoding nucleic acids may be isolated using conventional miniprep techniques, for example, using commercially available kits, e.g., Qiagen (Valencia, Calif.), after which encoding sequences may be identified by such steps as PCR amplification, DNA sequencing or hybridization to complementary sequences. In other embodiments, where epitopes are expressed from integrated vectors, epitope-encoding nucleic acids from isolated target cells may be amplified from the target cell genome by PCR, followed by isolation and analysis of the resulting amplicon, for example, by DNA sequencing. In the latter embodiments, epitope-encoding nucleic acids may be flanked by primer binding sites to facilitate such analysis.

A variety of DNA sequence analyzers are available commercially to determine the nucleotide sequences epitope-encoding nucleic acids recovered from target cells in accordance with the invention. Commercial suppliers include, but are not limited to, 454 Life Sciences, Life Technologies Corp., Illumina, Inc., Pacific Biosciences, and the like. The use of particular types DNA sequence analyzers is a matter of design choice, where a particular analyzer type may have performance characteristics (e.g., long read lengths, high number of reads, short run time, cost, etc.) that are particularly suitable for the experimental circumstances. DNA sequence analyzers and their underlying chemistries have been reviewed in the following references, which are incorporated by reference for their guidance in selecting DNA sequence analyzers: Bentley et al. (2008) Nature 456: 53-59; Margulies et al. (2005) Nature 437: 376-380; Metzker (2010) Nature Rev. Genet. 11:31-46; Fuller et al. (2009) Nat. Biotechnol. 27:1013-1023; Zhang et al. (2011) J. Genet. Genomics 38:95-109). Generally, epitope-encoding nucleic acids are extracted from target cells using conventional techniques and prepared for sequence analysis in accordance with manufacturer's instructions.

### VI. Uses and Methods

In addition, described herein are methods for screening libraries of target cells comprising candidate antigens for identifying antigens specific to cytotoxic lymphocytes (e.g., a cytotoxic T cell and/or NK cell). The methods include a) contacting an APC or a library of APCs described herein with one or more cytotoxic T cells and/or NK cells under conditions appropriate for recognition by the cytotoxic cell and/or NK cell of antigen presented by the cell or the library of cells; b) identifying APC(s) having an activated scramblase upon cleavage by the serine protease originating from the cytotoxic T cell and/or NK cell, and/or the caspase, in response to recognition by the cytotoxic T cell and/or NK cell of antigen presented by the cell or the library of cells; and c) determining the nucleic acid sequence encoding the antigen from the cell identified in step b), thereby identifying the antigen that is recognized by the cytotoxic T cell and/or NK cell. In some embodiments, the methods further comprise preparing a library of target cells as described herein prior to step a). In some embodiments, the APC(s) are intact, such as during one or more steps involving biophysical and/or analytical processing of cells (e.g., MHC-antigen expression by cells, contact of cells with other cells, detection of PS displayed by cells, PS-mediated cell binding, PS-mediated cell isolation, preparation for cellular nucleic acid isolation, and the like). As demonstrated below, APC(s) can be selected during a time period after reporter signal detection but before cytolysis and/or apoptosis has progressed to the point of cell destruction.

In some embodiments, phospholipid scramblase mediated by serine protease and/or caspase activity is used as a marker of the recognized APC. For example, GzB is a cytotoxic serine protease secreted by cytotoxic lymphocytes (e.g., a cytotoxic T cell and/or NK cell) into the recognized APC. GzB triggers caspase activation and apoptosis in the APC. Previous work demonstrated that the GzB released into target cells during cytolytic killing leads to complete proteolysis of the GzB targets, indicating robust enzymatic activity to serve as the basis of a reporter. To detect serine protease and/or caspase activity, such as GzB activity, an ordinarily skilled artisan may use a reporter of phospholipid scrambling such as those described herein. Such reporters are typically not activated by general apoptosis pathways, or are activated much later in general apoptosis pathways. For examples, in some embodiments, when target cells comprising the candidate antigens specifically bind their cognate T cells, the reporter of the target cell is activated and promotes the translation and exposure of PS, which enables Annexin-V based isolation or enrichment of the recognized target cells (e.g., by a Annexin-V bead/column).

In some embodiments, at least one additional reporter is used in combination with the reporters of phospholipid scrambling described herein. In some embodiments, the target cells described herein are engineered to contain at least one additional reporter gene construct which may express a reporter (e.g., luciferase, fluorescent protein, surface protein) upon antigen recognition by a T cell. The of skill in the art will recognize that other markers of the recognized APC may be used in combination with the reporters of phospholipid scramblase activity described herein, such as other serine proteases secreted by cytotoxic T lymphocytes (granzymes A, B, C, D, E, F, G, H, K, and M) or other enzymes or proteases such as TEV protease engineered into T cells to be secreted into target cells.

In some embodiments, the additional reporter is a fluorescent protein such as luciferase, red fluorescent protein, green fluorescent protein, yellow fluorescent protein, a green fluorescent protein derivative, or any engineered fluorescent protein. In further embodiments, detection of the fluorescent reporter may be detected using fluorescence techniques. For example, fluorescent protein expression may be measured using a fluorescence plate reader, flow cytometry, or fluorescence microscopy. In some embodiments, the activated target cells may be sorted based on expression of a fluorescent reporter using a fluorescence activated cell sorter (FACS).

In some embodiments, the additional reporter is a cell-surface marker. Target cells can upregulate or downregulate various cell surface markers upon engaging a TCR. In some embodiments, the level of expression of a cell surface protein such as CD80, CD86, MHC I, MHC II, CD11c, CD11b, CD8a, OX40-L, ICOS-1, or CD40 can change (e.g., increase or decrease after binding of a peptide antigen-major histocompatibility complex (pMHC) to a TCR. In some embodiments, detection of the cell surface reporter may be detected using techniques such as immunohistochemistry, fluorescence staining and quantification by flow cytometry, or assaying for changes in gene expression with cDNA arrays or mRNA quantification. In some embodiments, the activated target cells may be isolated based on expression of a cell surface reporter using magnetic activated cell sorting.

In some embodiments, the additional reporter is a reporter gene that encodes for a secreted factor such as IL6, IL-12, IFNα, IL-23, IL-1, TNF, or IL-10. In further embodiments, these secreted factors may be detected by mRNA quantification, cDNA arrays, or quantification of expressed proteins by assays such as an enzyme-linked immunosorbent assay (ELISA) or an enzyme linked immunospot (ELISPOT).

The marker of productive antigen recognition allows for an increased complexity of candidate antigens (i.e., the number of candidate antigens that may be included in the library where the single correct target of a T cell can successfully be identified) due to enhanced signal-to-noise. For example, unlike traditional methods of T cell receptor-antigen interaction analyses, the complexity of candidate antigens that may be assayed per 1 million target cells may be more than 1k (i.e., 1,000), 5k, 10k, 15k, 20k, 25k, 30k, 35k, 40k, 45k, 50k, 55k, 60k, 65k, 70k, 75k, 80k, 85k, 90k, 95k, 100k, 105k, 110k, 115k, 120k, 125k, 130k, 135k, 140k, 145k, 150k, 155k, 160k, 165k, 170k, 175k, 180k, 185k, 190k, 195k, 200k, 210k, 220k, 230k, 240k, 250k, 260k, 270k, 280k, 290k, 300k, 310k, 320k, 330k, 340k, 350k, 360k, 370k, 380k, 390k, 400k, 410k, 420k, 430k, 440k, 450k, 460k, 470k, 480k, 490k, 500k, 600k, 700k, 800k, 900k, 1000k, 1100k, 1200k, 1300k, 1400k, 1500k, 1600k, 1700k, 1800k, 1900k, 2000k, or more, or any range in between, inclusive (e.g., 100K to 2000K) target cells. In some antigen library formats, such as libraries of random peptides where each cell displays a unique peptide, antigens that may be screened are on the order of 1×10⁸ (i.e., hundreds of millions) to 1×10⁹ or more.

In addition to enhanced complexity of antigens that may be screened according to the compositions and methods described herein, the methods and compositions may also include APC that, in some embodiments, also include an inhibitor of DNA degradation (e.g., caspase-activated deoxyribonuclease (CAD)-mediated DNA degradation) in order to increase the efficiency of antigen recovery. Antigen(s) recognized by CTL of interest can be identified if they can be recovered from the modified APC marked by productive antigen recognition (e.g., obtaining the sequence of the exogenous nucleic acid encoding the cognate antigen bound by the T cell receptor). However, cytolysis induced by the CTL initiates degradation of DNA that hinders efficient recovery of antigen identities. Without inclusion of an inhibitor of DNA degradation, approximately one single antigen from 100 modified APC marked by productive antigen recognition (i.e., antigens that 1 out of 100 modified APC had been presenting or 1% efficiency) can be identified. As described further below, the inclusion of an inhibitor of DNA degradation, such as an inhibitor of CAD-mediated DNA degradation, increases the antigen recovery at least 5-fold (i.e., 5% efficiency) and may be at least 5%, 10%, 15%, 20%, 25%, 30%, 35%, 40%, 45%, 50%, or more, or any range in between, inclusive (e.g., 5%-50%) of antigen recovery. Thus, the present methods may be used to attain greater than 5%, e.g., 50% or higher recovery (with 100% being the theoretical limit).

Due to the large number of antigens that may be screened and efficiency of antigen recovery in an individual experiment, the methods described herein require fewer T cells and may therefore be applied to samples with limited numbers of T cells directly ex vivo.

The library of target cells may be incubated with cytotoxic T cells and/or NK cells under conditions that permit binding and recognition of apeptide antigen-major histocompatibility complex (pMHC) complex by T cell receptors of the cytotoxic T cells and/or NK cells. In some embodiments, target cells and cytotoxic T cells and/or NK cells are combined in a reaction mixture under conventional tissue culture conditions for mammalian cell culture. Such reaction mixtures may include conventional mammalian cell culture media, such as DMEM, RPMI, or like commercially available compositions, with or without additional components such as indicators and buffering agents to control pH and ionic concentrations, physiological salts, growth factors, antibiotics, and like compounds. Target cells and cytotoxic lymphocytes may be incubated for a period of time, e.g., 30 min to 24 hours, or in other embodiments, 30 min to 6 hours, under such conditions to permit cell-cell contact and receptor recognition; that is, where T cell receptors of cytotoxic lymphocytes specifically recognize pMHC complexes and generate an effector response that leads to the generation of a detectable signal in target cells.

In some aspects, T cells expressing a TCR of interest are cultured with target cells presenting a library of antigens on MHC molecules matching the host organism from which the TCR of interest was derived. In some embodiments, a T cell binds a target cell via engagement of pMHC complexes via the TCR, and results in expression of a reporter gene by the target cell, as described above. Activated target cells may be isolated using fluorescence activated cell sorting (FACS) or magnetic activated cell sorting (MACS). In some embodiments, antigenic peptides may be eluted off of the MHC molecule by treatment with an acid and/or reverse phase HPLC (RP-HPLC). In further embodiments, the antigenic peptide may be sequenced or analyzed by mass spectrometry. This method allows rapid and simultaneous screening of a large panel of target antigens against a TCR of interest, thereby allowing for accurate identification of the target antigen of a TCR.

In some embodiments, the method includes a step of quantitating a signal from the detectable label of the reporter molecule. In some embodiments, the method includes a step of enriching a population of the target cells based on the quantitated signal. In some embodiments, the method includes a step of introducing one or more mutations into one or more candidate antigen having the desired property.

In some embodiments, the methods further comprise enriching (for example, via PCR amplification) and identifying (for example, via sequencing) the antigens of interest in the sample. These steps may be carried out by a variety of techniques, such as, hybridization to microarrays, DNA sequencing, polymerase chain reaction (PCR), quantitative PCR (qPCR), pyrosequencing, next-generation sequencing (NGS), or like techniques. In some embodiments, the step of analyzing is carried out by sequencing the epitope-encoding nucleic acids. In other embodiments, the step of analyzing is carried out by amplifying the epitope-encoding nucleic acids from the isolated target cells, or a sample thereof, to form an amplicon, followed by DNA sequencing of member polynucleotides of the amplicon.

In some embodiments, the methods for screening as described herein are iterative. In some embodiments, the method includes iteratively repeating one or more of the screening steps described above, such as performing 1, 2, 3, 4, 5, or more rounds of screening. In some embodiments, APCs expressing a desired library of candidate antigen-encoding epitopes iteratively in order to enrich the library for epitopes yielding phospholipid scrambling reporter signal after each cycle. In some such embodiments, successive cycles may include the steps of contacting APCs to a sample comprising cytotoxic lymphocytes (e.g., a cytotoxic T cell and/or NK cell), identifying and/or selecting responding APCs, expanding the identified and/or selected isolated APCs. Epitope-encoding nucleic acids may be identified during any round or rounds of the iterative screening method, such as after the completion of several rounds, after a single round, or after non-consecutive rounds, as desired. In some embodiments, iterative screening may be performed until the number of epitope-encoding nucleic acids and/or clonotypes represented therein falls below a pre-determined number (e.g., enrichment for a desired number of clonotypes) and/or the frequencies of a pre-determined number of epitope-encoding nucleic acids identified rises above a pre-determined frequency (e.g., at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least 5%-20%).

In some embodiments, iterative screening may involve one or more steps of a) providing APCs comprising a reporter of phospholipid scrambling (and, optionally, further comprising one or more additional reporters of cytotoxic lymphocyte engagement with peptide antigen-major histocompatibility complex (pMHC) complexes expressed by the APCs) and candidate antigens for expression by the APCs in pMHC complexes, b) contacting the APCs with a sample comprising cytotoxic lymphocytes (e.g., cytotoxic T cells and/or NK cells) under conditions suitable for binding of the cytotoxic lymphocytes to pMHC complexes expressed by the APCs; c) selecting intact APCs generating a signal indicating recognition by a cytotoxic lymphocyte; d) identifying epitope-encoding nucleic acids from the selected APCs (such as by obtaining sequence information and/or by extracting the candidate epitope-encoding nucleic acids); e) generating an enriched library of epitope-encoding nucleic acids; f) repeating steps a) through e) with the enriched library of candidate epitope-encoding nucleic acids until a desired or pre-determined value, such as described herein, is determined. In some embodiments, the sequences of the epitope-encoding nucleic acids from the selected APCs are determined after any round of screening, after the final round of screening, or combination thereof.

An enriched library of epitope-encoding nucleic acids may be constructed as described herein for general libraries of epitope-encoding nucleic acids, such as by insertion of epitope-encoding nucleic acids of interest resulting from a screening round into an appropriate vector.

Compositions and methods described herein may be applied to T cells, NK cells, and any other cells that deliver a protease (e.g., granzyme) upon cell recognition. In some embodiments, the cytotoxic lymphocytes are cytotoxic T cells. These may be either CD4+ or CD8+. The cytotoxic T cells may express their endogenous receptors, or may be modified to express an exogenous antigen receptor of interest. In some embodiments, the exogenous receptor is from a T cell that does not have cytotoxic activity (e.g., non-cytotoxic CD4 T cell). The specificity of a T cell is contained in the sequence of its T cell receptor. It has been demonstrated that introducing the TCR from one T cell into another may retain the effector functions of the recipient cell while transferring the specificity of the new TCR. This is the basis of TCR therapeutics in general. Moreover, a TCR from a CD8 T cell can drive the effector functions of CD4 T cells when introduced into donor CD4 cells (Ghorashian et al. (2015) J. Immunol. 194:1080-1089). As demonstrated herein, transferring the TCR from a CD4 T cell into donor CD8 cells may confer GzB-mediated cytotoxic activity towards antigens presented on MHC class II and recognized by the CD4 TCR. In some embodiments, the exogenous T cell receptor is from a T helper (Th1 or Th2) or a regulatory T cell. Other types of cytotoxic cells may be used in the assays, such as natural killer cells, to identify factors those cells recognize. The cytotoxic lymphocytes used in the method may be clonal or a mixed population. Alternatively, or in addition, to CTLs, natural killer (NK) cells that have been engineered to express a T cell receptor may be used.

The cytotoxic T cells and/or NK cells may be obtained from a variety of sources. Reagents to identify and isolate human lymphocytes and subsets thereof are well known and commercially available. Lymphocytes for use in methods described herein may be isolated from peripheral blood mononuclear cells, or from other tissues in a human. In some embodiments, lymphocytes are taken from lymph nodes, a mucosal tissue (e.g., nose, mouth, bronchial tissue, tracheal tissue, the gastrointestinal tract, the genital tract (e.g., vaginal tissue), or associated lymphoid tissue), peritoneal cavity, spleen, thymus, lung, liver, kidney, neuronal tissue, endocrine tissue, peritoneal cavity, bone marrow, or other tissues. In some embodiments, cells are taken from a tissue that is the site of an active immune response (e.g., an ulcer, sore, or abscess). Cells may be isolated from tissue removed surgically, via lavage, or other means.

In some embodiments, the cytotoxic lymphocytes (e.g., cytotoxic T lymphocytes) or NK cells are isolated from a biological sample.

A “biological sample” refers to a fluid or tissue sample of interest that comprises cells of interest such as cytotoxic lymphocytes or antigen presenting cells. In exemplary embodiments, the biological sample comprises cytotoxic T cells (CTLs) and/or NK cells. A biological sample may be obtained from any organ or tissue in the individual, provided that the biological sample comprises cells of interest. The organ or tissue may be healthy or may be diseased. In some embodiments, the biological sample is from a location of autoimmunity, a site of autoimmune reaction, a tumor infiltrate, a virus infection site, or a lesion.

In some embodiments, a biological sample is treated to remove biological particulates or unwanted cells. Methods for removing cells from a blood or other biological sample are well known in the art and may include e.g., centrifugation, ultrafiltration, immune selection, or sedimentation etc. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, mouth mucosa sample, a cancer sample, a tumor sample, tumor infiltrate, a tissue sample (e.g., skin), a cell sample, a synovial fluid sample, or a combination of such samples. For the methods described herein, in some embodiments, a biological sample is blood or tissue biopsies (e.g., tumors, site of autoimmunity or other pathology).

The present invention provides methods for treatment of a subject in need thereof with therapeutics against the identified target antigens. Applications encompassed by the present invention include identifying T cell-antigen interaction in any circumstance in health or disease where such interaction is an in situ immune response, including, but not limited to, the circumstances of cancer, organ rejection, graft versus host disease, autoimmunity, chronic infection, vaccine response, and the like.

In some embodiments, methods encompassed by the present invention may be used to identify antigens in tumors that TILs recognize. Such antigen identity may inform cancer vaccine design or selection of the best tumor reactive T cells for autologous cell therapy. T cell clones from tumor infiltrates have been isolated and TCR sequencing of tumor infiltrates has demonstrated oligoclonal expansions of tumor-specific T cells. Patient-specific neoantigen libraries may be generated containing the novel protein fragments arising from somatic mutations in patient tumors. Tumor-specific T cells may then be screened systematically for recognition of these neoepitopes and screened genome-wide for recognition of non-mutated tumor antigens.

In some embodiments, methods encompassed by the present invention may be used to improve tissue matching between donors and recipients. Even in HLA matched donors and recipients there is organ rejection and the necessity of recipient immunosuppression. Rejection is mediated by “minor antigens” presented by the graft. Minor antigens are essentially the T cell peptide epitopes that have amino acid sequence differences arising from SNPs in the donor genome that are different from the recipients SNPs. Methods encompassed by the present invention may be used to identify the minor antigens that trigger recipient T cell responses. Likewise, in graft-versus-host disease, methods encompassed by the present invention may be used to identify the minor antigens in a recipient that trigger donor T cell responses.

With regard to autoimmunity (e.g., multiple sclerosis, Crohn's disease, rheumatoid arthritis, type I diabetes, and the like), method encompassed by the present invention may be used to identify underlying T cell antigens in the affected tissues which information, in turn, may be used to tolerize or deplete the reactive T cells causing the pathology. For example, it may be used to screen bulk T cells isolated from type 1 diabetes patients to identify the complete set of pancreatic autoantigens recognized by patient T cells.

In some embodiments, methods encompassed by the present invention may be used to identify viral antigens and to generate optimized vaccines and T cell therapies in infectious diseases (e.g., HIV, cytomegalovirus infection, and malaria). For example, there is a strong association between the MHC class I allele HLA-B57 and elite control of HIV, implicating CD8 T cells and specific target antigens as likely determinants of viral control. The technology disclosed herein may be used to systematically profile CU specificity in patients with particular clinical outcomes, for example immunity to controlled malaria exposure or elite control of HIV, to identify correlates of protection and inform vaccine design.

In some embodiments, compositions and methods are provided useful for diagnostic and prognostic uses. For example, APCs described herein may express antigens of interest (e.g., antigens from one or more virus, bacteria, fungi, protozoa, helminth, multicellular parasitic organism, cancer target, and the like) against which the presence, absence, and/or amount of recognition by a sample comprising cytotoxic lymphocytes (e.g., cytotoxic T cells and/or NK cells) are determined. Such embodiments are useful for a number of uses, such as determining immunity against the antigens of interest in a subject from which the sample was derived. Thus, the screening methods described herein can be applied using APCs expressing pre-determined antigens of interest in order to determine the presence, absence, and/or amount of recognition of the APCs by the subject's cytotoxic lymphocytes (e.g., cytotoxic T cells and/or NK cells) and numerous representative embodiments are described herein (e.g., MHC matching, intact cell separation, epitope-encoding nucleic acid sequencing, etc.). The amount of recognition can be determined as described herein, for example, by determining the frequency of APCs providing reporter signals, the frequency of epitope-encoding nucleic acid sequences resulting from APCs providing reporter signals, and the like.

The herein described technology may be applied to identify the specificities of mixed populations of T cells. This allows the characterization of protective or pathogenic T cell responses even in cases where specific clones or TCRs of interest have not yet been identified.

### VII. Kits

The present invention also encompasses kits. For example, the kit may comprise reporters of phospholipid scrambling described herein, nucleic acids and/or vectors encoding reporters of phospholipid scrambling described herein described herein, modified cells comprising reporters of phospholipid scrambling described herein, and combinations thereof, packaged in a suitable container and may further comprise instructions for using such reagents. The kit may also contain other components, such as nucleic acids or vectors encoding a library of candidate antigens, cytotoxic T cells, NK cells, reagents useful for detecting PS (e.g., Annexin-V beads and/or Annexin-V column), and/or screening plates or tools packaged in a the same or separate container.

The disclosure is further illustrated by the following examples, which should not be construed as limiting.

### EXAMPLES

### Example 1: Materials and Methods for Example 2

a. XKR8 Granzyme Reporter Cloning

gBlock DNA fragments encoding XKR-8 GZMB reporter (hXKR8-GZMB, YW3) and XKR-8-GZMB with GS linker (LGB-XKR8, YW1) were synthesized by IDT DNA. The reporters were cloned into a lentiviral vector containing a Thy1.1 selection maker (pHAGE-EF1a-MCa-UBC-Th1) via restriction digest and ligation. The product reporter constructs YW1 and YW3 were sequence-confirmed and packaged into lentivirus for transduction.

b. Cell Line Generation

As described herein, a GZM-IFP reporter has been developed to measure pMHC-TCR mediated T cell killing of engineered target cells such as engineered HEK 293 cells. Here. YW1 and YW3 were introduced to HLA-A2-expressing HEK 293 reporter cells expressing IFP-GZM reporter by lentiviral transduction. The transduced cells were sorted by Thy1.1+ staining.

c. Killing Assay

Control HLA-A2 IFP reporter cells, HLA-A2 IFP YW1, and HLA-A2 IFP YW3 cells were labeled with CellTrace™ Violet (Invitrogen Cat. #C34557), and plated in 6-well plates at 250K cells per well density and cultured overnight. The next morning selected wells were pulsed with 1 uM NLVPMVATVQ peptide for 1 hour. CIV TCR-T cells targeting the NLVPMVATVQ w ere added to the wells at 250K cells per well and co-cultured with reporter cells for 1 to 4 hours. When harvesting, cells were stained with Annexin-V-PE for PS detection and analyzed for PE and IFP double staining.

d. Annexin Enrichment for Screening

Following co-culture, cells were harvested, centrifuged, and washed with 100 ml Annexin V binding buffer (Milteny). Cells were centrifuged then resuspended in a mix of Annexin V binding buffer+beads (1E8 cells/ml total volume with 200 ul Annexin V beads/1E8 cells). The cell-bead mixture was incubated at room temperature for 15 minutes, then 100 ml of Annexin V binding buffer was added and the mixture was centrifuged. The cell-bead pellet was resuspended in 30 ml Annexin V buffer, passed through a 70 um filter (Corning) and applied to an AutoMACS instrument (Milteny) for magnetic bead binding and Annexin V+ cell separation. Selected cells were collected for further processing by FACS. An aliquot of the initial cell mixture, the flow-through and the selected cells from the magnetic separation were collected for quality control (QC) analysis.

### Example 2: Engineered Scramblase Allows Efficient Annexin V-Based Enrichment of Target Cells

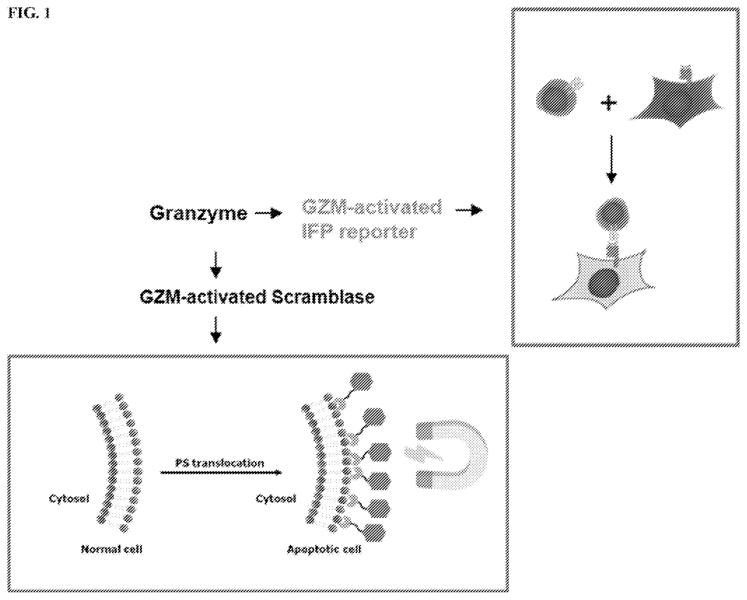

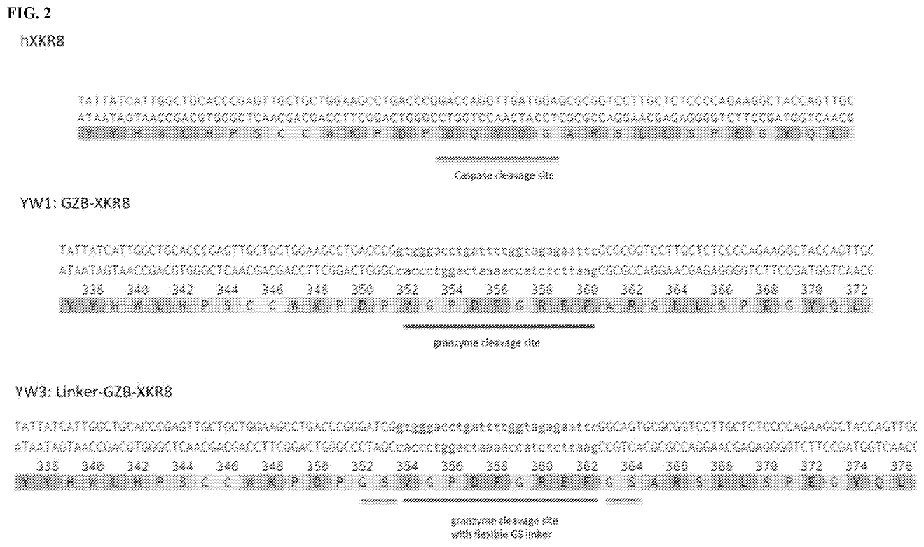

The granzyme-activated IFP reporter has previously been reported in U.S. Pat. Publ. 2020/0102553 and Kula et al. (2019) Cell 178:1016-1028. Here, a representative granzyme-activated scramblase reporter is provided, which enhances the presentation of PS on target cells upon T cell or NK cell recognition, and enables efficient purification of these cells with Annexin V columns (FIG. 1). The scramblase reporter constructs with engineered granzyme B cleavage sites are shown in FIG. 2.

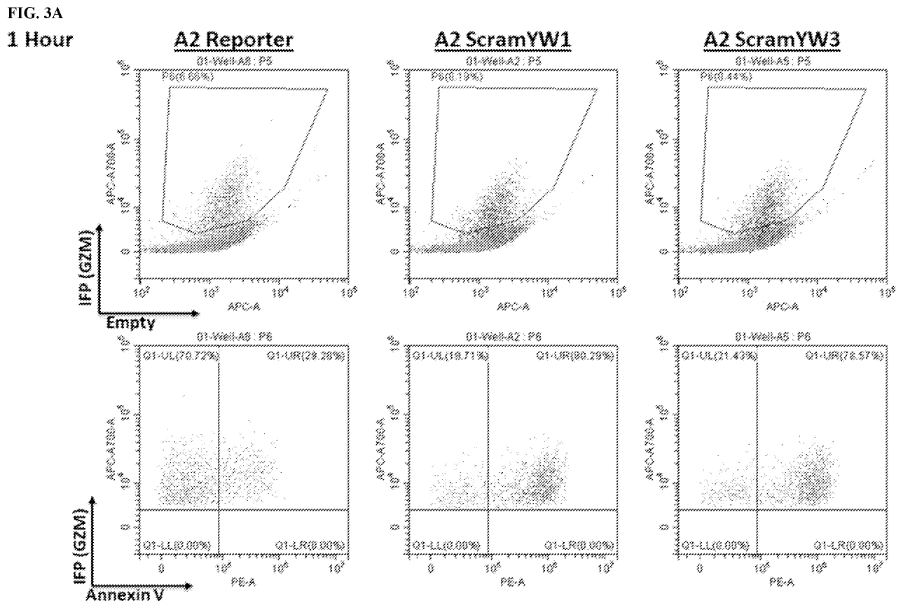

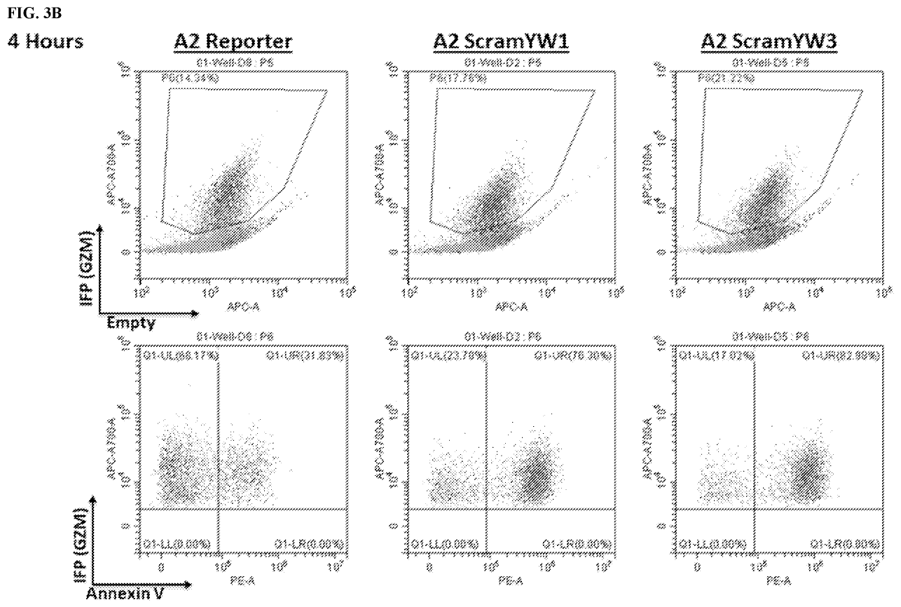

It was found that scramblase enhances Annexin V staining following T cell recognition (FIGS. 3A and 3B). YW1 and YW3 were introduced into HLA-A2 IFP-GzB reporter cells, and pulsed with a CMV peptide. Pulsed HLA-A2 IFP-GzB reporter cells without scramblase were used as control. After co-culture with CMV-specific T cells for 1 hour or 4 hours, reporter cells became IFP positive, indicating T cell mediated killing. Cells were also measured for PS level by Annexin V staining. In cells expressing scramblase, the Annexin and IFP double-positive population increased from 29-32% to 76-82%, indicating that the scramblase introduction reduces the IFP+ cell loss during Annexin enrichment approximately three-fold.

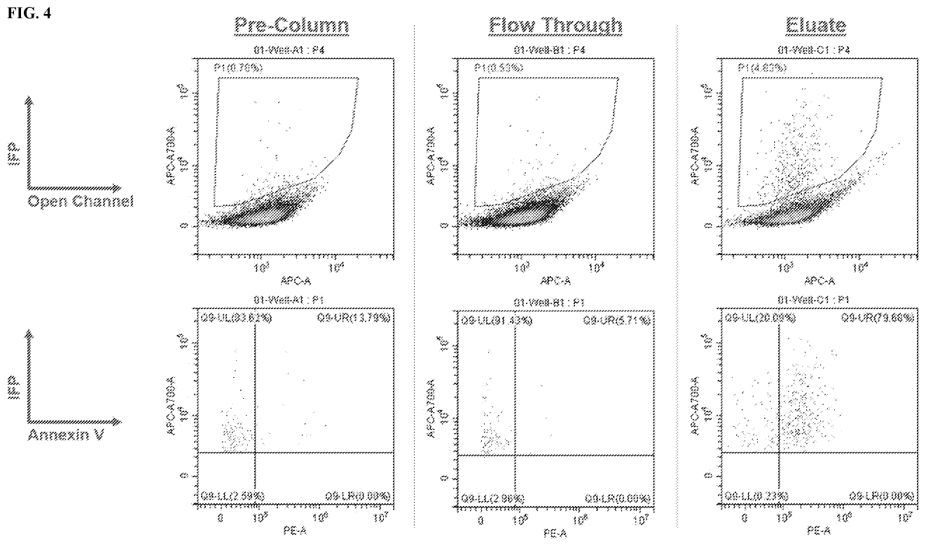

Annexin V column-based enrichment of YW3 granzyme scramblase/IFP-GzB double reporter cells in the context of a large scale screen was tested. The target cells engaged by T cells were IFP positive. As shown in FIG. 4, the percentage of IFP-positive cells increased from 0.78% to 4.83% after Annexin V column enrichment of the scramblase/IFP reporter cells, indicating that the engineered scramblase allowed efficient annexin-based enrichment of IFP+ target cells. The lower panel of FIG. 4 shows that eluate cells exhibited elevated levels of both Annexin-V and IFP signal.

Thus, representative engineered non-fluorescent reporters that allow for the identification of target cells recognized by T cells are described. These exemplary, non-limiting reporters work through a cell membrane composition change based on the use of apoptosis-mediated scramblase (e.g., XKR family members like human scramblase hXKR8). Synthetic scramblase reporter genes in which the native caspase cleavage site is replaced by a granzyme B cleavage site with or without additional GS linkers were developed. Once introduced to mammalian cells, these reporter genes allow a target cell recognized by cytotoxic T cells to be detected by an increase of cell surface PS level. These reporters may be used independently or in combination with other reporters to identify cells targeted by T cells for the purpose of TCR antigen discovery.

Unlike existing fluorescent or cytoplasmic granzyme reporters, the engineered scramblase reporters cause a specific change at cellular membranes, such as the cell surface membrane. This allows large-scale, rapid purification (e.g., using binding agents like beads, plates, columns, etc.) and subsequent detection of cell populations engaged by cytotoxic T cells. For example, IFP-reporter-based cell sorting has been utilized for genome-wide T-Scan screens to identify TCR antigens. In conventional screens, a large number (200 million to 1.2 billion) of cells need to be sorted by flow cytometry. The pre-enrichment of apoptotic target cells by Annexin-V based purification may enrich the IFP reporter cells targeted by T cells and reduce the number of cells for sorting. However, when using unmodified target cells, this purification step results in significant cell loss. This is because of the abundance of serine protease (e.g., GzB)-positive (meaning recognized by a cytotoxic T cell and/or NK cell), Annexin V-negative target cells that fail to be captured in the Annexin-V columns. Specifically, PS exposure occurs downstream of caspase activation during apoptosis, whereas cytotoxic payloads from recognition by cytotoxic T cells and/or NK cells (e.g., GzB activity) is maximal immediately following the delivery of cytotoxic granules, prior to the onset of apoptosis. The use of the phospholipid scrambling reporter addresses this issue by synchronizing the presentation of PS, which is now triggered directly by the serine protease activity, and the activation of other reporters, such as granzyme reporters. Moreover, the use of the phospholipid scramblase reporter enhances the strength of PS signal upon T cell recognition. This allows for more efficient capture of target cells when using Annexin V purification alone or in combination with other reporters. Collectively, the use of phospholipid scramblase reporters results in more efficient and earlier PS presentation by target cells recognized by T cells. This, in turn, greatly enhances the performance of column-based Annexin V pre-enrichment steps and enables antigen discovery at a higher scale and efficiency.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. A cell comprising a reporter of phospholipid scrambling, wherein the reporter of phospholipid scrambling comprises a scramblase comprising a serine protease cleavage site and/or a caspase cleavage site that activates the scramblase upon cleavage by the serine protease and/or the caspase.

2. The cell of claim 1, wherein the activated scramblase is capable of promoting the translocation of phosphatidylserine (PS) to the outer leaflet of a cell membrane lipid bi-layer.

3. The cell of claim 2, wherein the cell membrane lipid bi-layer is the cell surface membrane bi-layer.

4. The cell of any one of claims 1-3, wherein the serine protease cleavage site and/or the caspase cleavage site is comprised within the scramblase using one or more linkers, optionally wherein the linker is a glycine-serine (GS) linker.

5. The cell of any one of claims 1-4, wherein the GzB cleavage site is flanked on each side by a linker, optionally wherein the linker is a GS linker.

6. The cell of any one of claims 1-5, wherein the serine protease is a granzyme, optionally wherein the granzyme is selected from the group consisting of granzyme A, B, C, D, E, F, G, H, K, and M.

7. The cell of claim 6, wherein the granzyme cleavage site has a sequence selected from the group consisting of granzyme cleavage sites listed in Table 1A.

8. The cell of any one of claims 1-7, wherein the caspase is an apoptosis-mediated caspase, optionally wherein the caspase is selected from the group consisting of caspase 3, 6, 7, 8, and 9.

9. The cell of claim 8, wherein the caspase cleavage site has a sequence selected from the group consisting of caspase cleavage sites listed in Table 1B.

10. The cell of any one of claims 1-9, wherein the scramblase does not comprise a caspase cleavage site that activates the scramblase upon cleavage by the caspase.

11. The cell of any one of claims 1-10, wherein the scramblase is an apoptosis-mediated scramblase.

12. The cell of claim 11, wherein the apoptosis-mediated scramblase is Xkr8, Xkr4, Xkr9, Xkr3, or an ortholog thereof, optionally wherein the apoptosis-mediated scramblase is human Xkr8 (hXkr8), human Xkr4 (hXkr4), or human Xkr9 (hXkr9).

13. The cell of any one of claims 1-12, wherein the reporter comprises an amino acid sequence having at least 80% identity with SEQ ID NO: 2 or 6.

14. The cell of any one of claim 1-13, wherein the cell further comprises at least one additional reporter of contact with cytotoxic lymphocytes, optionally wherein the reporter indicates peptide antigen-major histocompatibility complex (pMHC) complex-mediated contact of the cell with a pMHC complex-binding receptor expressed by the cytotoxic lymphocyte, and further optionally wherein the cytotoxic lymphocyte is a cytotoxic T cell and the receptor is a T cell receptor (TCR).

15. The cell of claim 14, wherein the at least one additional reporter comprises a granzyme-activated infrared fluorescent protein (IFP) comprising a granzyme cleavage site that activates the IFP fluorescence upon cleavage by the granzyme, optionally wherein a) the reporter and the at least one additional reporter are comprised on the same construct and/or b) the granzyme is granzyme B.

16. The cell of any one of claims 1-15, wherein the reporter and/or the at least one reporter further comprises gene expression element(s) that is capable of expressing the reporter protein, optionally wherein the gene expression element comprises a promoter operably linked to the nucleic acid encoding the reporter protein.

17. The cell of any one of claims 1-16, wherein the reporter and/or the at least one reporter further comprises a selection marker, optionally wherein the selection marker is Thy1.1.

18. The cell of any one of claims 1-17, wherein the reporter and/or at least one reporter is flanked on each side by pre-determined primer recognition sequences.

19. The cell of any one of claims 1-18, wherein the reporter and/or the at least one reporter is stably introduced into the genome of the cell, optionally wherein the stable introduction is via a lentiviral vector, a retroviral vector, or a transposon.

20. The cell of any one of claims 1-19, wherein the cell is a primary cell or a cell of a cell line.

21. The cell of any one of claims 1-20, wherein the cell is a professional antigen presenting cell (APC), optionally wherein the APC is selected from the group consisting of a dendritic cell, a macrophage, a langerhan cell, and a B cell.

22. The cell of any one of claims 1-21, wherein the cell does not express an endogenous MHC molecule and is engineered to express an exogenous MHC molecule.

23. The cell of any one of claims 1-22, wherein caspase-activated deoxyribonuclease (CAD)-mediated DNA degradation is blocked in the cell, optionally wherein the cell further comprises an exogenous inhibitor of CAD-mediated DNA degradation, a CAD knockout, or a caspase knockout.

24. The cell of claim 23, wherein the exogenous inhibitor of CAD-mediated DNA degradation is a nucleic acid encoding inhibitor of caspase-activated deoxyribonuclease (ICAD) gene in expressible form, an inhibitory nucleic acid targeting CAD or caspase 3, a small molecule inhibitor of caspase 3, a chemical DNAse inhibitor, or a peptide or protein inhibitor of caspase 3, optionally wherein the ICAD gene is a caspase-resistant ICAD mutant and/or the caspase knockout is a caspase 3 knockout.

25. The cell of any one of claims 1-24, wherein the cell further comprises an exogenous nucleic acid encoding one or more candidate antigens, optionally wherein a) the one or more candidate antigens are comprised on the same construct as the reporter, b) one or more candidate antigens are comprised on the same construct as the at least one additional reporter, or c) the one or more candidate antigens are comprised on the same construct as the construct comprising the reporter and the at least one additional reporter.

26. The cell of claim 25, wherein the exogenous nucleic acid further comprises gene expression element(s) that is capable of expressing the one or more candidate antigens, optionally wherein the gene expression element comprises a promoter operably linked to the nucleic acid encoding the one or more candidate antigens.

27. The cell of claim 25 or 26, wherein the exogenous nucleic acid further comprises a selection marker, optionally wherein the selection marker is a drug resistance marker.

28. The cell of any one of claims 25-27, wherein the exogenous nucleic acid is flanked on each side by pre-determined primer recognition sequences.

29. The cell of any one of claims 25-28, wherein the exogenous nucleic acid is stably introduced into the genome of the cell, optionally wherein the stable introduction is via a lentiviral vector, a retroviral vector, or a transposon.

30. The cell of any one of claims 25-29, wherein the one or more candidate antigens are expressed and presented by the cell with MHC class I or MHC class II molecules.

31. The cell of any one of claims 25-30, wherein the one or more candidate antigens is up to 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids in length.

32. The cell of any one of claims 25-30, wherein the one or more candidate antigens is greater than 300 amino acids in length.

33. The cell of any one of claims 25-32, wherein the exogenous nucleic acid encoding a candidate antigen is derived from an infectious organism, optionally wherein the infectious organism is selected from the group consisting of a virus, a bacteria, a fungi, a protozoa, a helminth, and a multicellular parasitic organism.

34. The cell of any one of claims 25-33, wherein the exogenous nucleic acid encoding a candidate antigen is derived from a human DNA, optionally wherein the human DNA is obtained from a cancer cell.

35. A library of cells of any one of claims 1-34, wherein the cells comprise different exogenous nucleic acids encoding one or more candidate antigens to thereby represent a library of candidate antigens expressed and presented with MHC class I and/or MHC class II molecules.

36. The library of claim 35, wherein a cell of the library expresses more than one candidate antigen.

37. The library of claim 35, wherein a cell of the library expresses one candidate antigen.

38. The library of any one of claims 35-37, wherein the library of cells comprises from about 10² to about 10¹⁴ individual candidate antigens.

39. The library of any one of claims 35-38, wherein the library of cells comprises from about 10² to about 10¹⁴ cells.

40. The library of any one of claims 35-39, wherein the library of cells comprises less than 20% of cells lacking an exogenous nucleic acid encoding one or more candidate antigens.

41. A reporter of phospholipid scrambling comprising a scramblase comprising a serine protease cleavage site and/or a caspase cleavage site that activates the scramblase upon cleavage by the serine protease and/or the caspase.

42. The reporter of claim 41, wherein the activated scramblase is capable of promoting the translocation of phosphatidylserine (PS) to the outer leaflet of a cell membrane lipid bi-layer.

43. The reporter of claim 42, wherein the cell membrane lipid bi-layer is the cell surface membrane bi-layer.

44. The reporter of any one of claims 41-43, wherein the serine protease cleavage site and/or the caspase cleavage site is comprised within the scramblase using one or more linkers, optionally wherein the linker is a glycine-serine (GS) linker.

45. The reporter of any one of claims 41-44, wherein the GzB cleavage site is flanked on each side by a linker, optionally wherein the linker is a GS linker.

46. The reporter of any one of claims 41-45, wherein the serine protease is a granzyme, optionally wherein the granzyme is selected from the group consisting of granzyme A, B, C, D, E, F, G, H, K, and M.

47. The reporter of claim 46, wherein the granzyme cleavage site has a sequence selected from the group consisting of granzyme cleavage sites listed in Table 1A.

48. The reporter of any one of claims 41-47, wherein the caspase is an apoptosis-mediated caspase, optionally wherein the caspase is selected from the group consisting of caspase 3, 8, and 9.

49. The reporter of claim 48, wherein the caspase cleavage site has a sequence selected from the group consisting of caspase cleavage sites listed in Table 1B.

50. The reporter of any one of claims 41-49, wherein the scramblase does not comprise a caspase cleavage site that activates the scramblase upon cleavage by the caspase.

51. The reporter of any one of claims 41-50, wherein the scramblase is an apoptosis-mediated scramblase.

52. The reporter of claim 51, wherein the apoptosis-mediated caspase is Xkr8, Xkr4, Xkr9, Xkr3, or an ortholog thereof, optionally wherein the apoptosis-mediated caspase is human Xkr8 (hXkr8), human Xkr4 (hXkr4), human Xkr9 (hXkr9), or human Xkr3 (hKxr3).

53. The reporter of any one of claims 41-52, wherein the reporter comprises an amino acid sequence having at least 80% identity with SEQ ID NO: 2 or 6.

54. The reporter of any one of claim 41-53, wherein the reporter further comprises at least one additional reporter of contact with cytotoxic lymphocytes, optionally wherein the reporter indicates peptide antigen-major histocompatibility complex (pMHC) complex-mediated contact of the cell with a pMHC complex-binding receptor expressed by the cytotoxic lymphocyte, and further optionally wherein the cytotoxic lymphocyte is a cytotoxic T cell and the receptor is a T cell receptor (TCR).

55. The reporter of claim 54, wherein the at least one additional reporter comprises a granzyme-activated infrared fluorescent protein (IFP) comprising a granzyme cleavage site that activates the IFP fluorescence upon cleavage by the granzyme, optionally wherein a) the reporter and the at least one additional reporter are comprised on the same construct and/or b) the granzyme is granzyme B.

56. The reporter of any one of claims 41-55, wherein the reporter further comprises an exogenous nucleic acid encoding one or more candidate antigens.

57. The reporter of any one of claims 41-56, wherein the one or more candidate antigens are expressed and presented by MHC class I or MHC class II molecules.

58. The reporter of any one of claims 41-57, wherein the one or more candidate antigens is up to 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids in length.

59. The reporter of any one of claims 41-58, wherein the one or more candidate antigens is greater than 300 amino acids in length.

60. The reporter of any one of claims 41-59, wherein the exogenous nucleic acid encoding a candidate antigen is derived from an infectious organism, optionally wherein the infectious organism is selected from the group consisting of a virus, a bacteria, a fungi, a protozoa, a helminth, and a multicellular parasitic organism.

61. The reporter of any one of claims 41-60, wherein the exogenous nucleic acid encoding a candidate antigen is derived from a human DNA, optionally wherein the human DNA is obtained from a cancer cell.

62. The reporter of any one of claims 41-61, wherein the reporter, the at least one additional reporter, and/or the exogenous nucleic acid further comprises gene expression element(s) capable of expressing the reporter protein(s) and candidate antigen(s), optionally wherein the gene expression element(s) comprises a promoter operably linked to the nucleic acid encoding the reporter protein(s) and the candidate antigen(s).

63. The reporter of any one of claims 41-62, wherein the reporter, the at least one additional reporter, and/or the exogenous nucleic acid further comprises a selection marker, optionally wherein the selection marker is Thy1.1 and/or a drug resistance marker.

64. The reporter of any one of claims 41-63, wherein the reporter, the at least one additional reporter, and/or the exogenous nucleic acid is flanked on each side by pre-determined primer recognition sequences.

65. The reporter of any one of claims 41-64, wherein the reporter is stably introduced into the genome of the cell, optionally wherein the stable introduction is via a lentiviral vector, a retroviral vector, or a transposon.

66. A nucleic acid that encodes the reporter of any one of claims 41-65, optionally wherein the nucleic acid comprises a nucleotide sequence having at least 80% identity with the nucleic acid sequence of SEQ ID NO: 1 or 5.

67. A vector that comprises the nucleic acid of claim 66, optionally wherein the vector is a cloning vector, an expression vector, or a viral vector.

68. The vector of claim 67, wherein the vector further comprises a nucleic acid that encodes a selection marker, optionally wherein the selection marker is Thy1.1 or a drug resistance marker.

69. A cell that comprises the nucleic acid or vector of any one of claims 55-68.

70. A method of making a recombinant cell comprising (i) introducing in vitro or ex vivo a recombinant nucleic acid or a vector of any one of claims 55-68 into a host cell, (ii) culturing in vitro or ex vivo the recombinant host cell obtained, and (iii), optionally, selecting the cells which express said recombinant nucleic acid or vector.

71. A system for detection of an antigen presented by an antigen presenting cell (APC) that is recognized by a cyotoxic lymphocyte, optionally wherein the cyototoxic lymphocyte is a cytotoxic T cell and/or natural killer (NK) cell, comprising:
a) an APC comprising a cell of any one of claims 25-34; and
b) a cytotoxic lymphocyte.

72. The system of claim 64, wherein the APC is comprised within a library of cells of any one of claims 35-40.

73. The system of claim 71 or 72, wherein a) the cytotoxic T cell and/or NK cell and b) the APC are MHC matched.

74. The system of any one of claims 71-73, wherein the cytotoxic ‘I’ cell and/or NK cell are modified to express an antigen receptor that is matched to the MHC expressed by the APC.

75. The system of any one of claims 71-74, wherein a) the cytotoxic T cell and/or NK cell and b) the APC are autologous relative to the source of the cells.

76. The system of any one of claims 71-75, wherein the cytotoxic T cell and/or NK cell are modified to express a T cell receptor from a non-cytotoxic CD4+ T cell.

77. The system of any one of claims 71-76, wherein the cytotoxic T cell toxic CD4+ T cell or a cytotoxic CD8+ T cell.

78. A method for identifying an antigen that is recognized by a cyotoxic T cell and/or NK cell, comprising:
a) contacting an APC or a library of APCs of any one of claims 1-40 with one or more cytotoxic lymphocytes, optionally wherein the cytotoxic lymphocytes are cytotoxic T cells and/or NK cells, under conditions appropriate for recognition by the cytotoxic lymphocytes of antigen presented by the APC or the library of APCs;
b) identifying APC(s) having an activated scramblase upon cleavage by the serine protease originating from a cytotoxic lymphocyte, and/or the caspase, in response to recognition by the cytotoxic lymphocyte of antigen presented by the cell or the library of cells; and
c) determining the nucleic acid sequence encoding the antigen from the cell identified in step b), thereby identifying the antigen that is recognized by the cytotoxic lymphocyte.

79. The method of claim 78, wherein the APC(s) having an activated scramblase is detected by directly or indirectly detecting activated scramblase activity.

80. The method of claim 79, wherein activated scramblase activity is identified by detecting translocation of phosphatidylserine (PS) to the outer leaflet of a cell membrane lipid bi-layer.

81. The method of claim 80, wherein the cell membrane lipid bi-layer is the cell surface membrane bi-layer.

82. The method of claim 80 or 81, wherein PS is detected using an Annexin V binding assay.

82. The method of claim 78 or 79, wherein activated scramblase activity is identified by detecting scramblase cleaved by the serine protease and/or the caspase.

83. The method of any one of claims 78-82, wherein step b) further comprises isolating cells having an activated scramblase, optionally wherein the cells are isolated using affinity purification or fluorescence-activated cell sorting (FACS).

84. The method of any one of claims 78-83, wherein step c) comprises nucleic acid amplification, optionally wherein nucleic acid is amplified using polymerase chain reaction (PCR).

85. The method of any one of claims 78-84, wherein the sequencing is by pyrosequencing or next-generation sequencing.

86. The method of any one of claims 78-85, wherein step b) or step c) further comprises generating an APC or a library of APCs of any one of claims 1-40 that expresses the nucleic acid sequence encoding antigens from APCs obtained from the cell(s) having an activated scramblase upon cleavage by the serine protease and/or the caspase.

87. The method of claim 86, further comprising repeating steps a) and b) until the cell(s) having an activated scramblase upon cleavage by the serine protease and/or the caspase reaches a desired proportion of the total APCs, optionally wherein the proportion is greater than or equal to at least 0.5% of the total population of APCs.

88. The method of any one of claims 78-87, wherein the library of cells comprises at least 100 different candidate antigens.

89. The method of any one of claims 78-88, wherein the cytotoxic lymphocytes and/or APCs are autologous relative to the source of the cells.

90. The method of any one of claims 78-89, wherein the source of the cells is selected from the group consisting of blood, tumor, healthy tissue, ascites fluid, location of autoimmunity, tumor infiltrate, virus infection site, lesion, mouth mucosa, and skin of a subject.

91. The method of any one of claims 78-90, wherein the source of the cells is a site of infection or autoimmune reactivity in a subject.

92. The method of any one of claims 78-91, wherein the cytotoxic lymphocytes are cytotoxic T cells, optionally wherein the cytotoxic T cells are cytotoxic CD4+ T cells and/or CD8+ T cells.

93. The method of any one of claims 78-92, wherein the cytotoxic lymphocytes are modified to express a T cell receptor from a non-cytotoxic CD4+ T cell.

94. The method of any one of claims 78-93, wherein a) the cytotoxic lymphocytes and b) the APC are MHC matched.

95. The method of any one of claims 78-94, wherein the cytotoxic lymphocytes are modified to express an antigen receptor that is matched to the MHC expressed by the APC.

96. The cell, system, or method of any one of claims 1-95, wherein the source of the cells is a mammal, optionally wherein the mammal is a rodent, a primate, or a human.
